(19) United States
(12) Patent Application Publication
(10) Pub. No.: US 2024/0209029 A1
(43) Pub. Date: Jun. 27, 2024

(54) ADIPONECTIN GLYCOPEPTIDES AND COMPOSITIONS AND METHODS OF USE THEREOF

(71) Applicant: The University of Hong Kong, Hong Kong (CN)

(72) Inventors: Yu WANG, Hong Kong (CN); Xuechen LI, Hong Kong (CN); Aimin XU, Hong Kong (CN); Hongxiang WU, Hong Kong (CN); Yiwei ZHANG, Hong Kong (CN); Yuanxin LI, Hong Kong (CN)

(21) Appl. No.: 18/555,201

(22) PCT Filed: Apr. 21, 2021
(86) PCT No.: PCT/IB2021/053309

§ 371 Date: Oct. 12, 2023

(51) Int. Cl. C07K 14/00; A61K 38/00; A61P 3/04; A61P 3/10; A61P 35/00; B01J 23/50; C07K 1/107; C07K 14/575

(52) U.S. Cl. CPC C07K 14/001; A61P 3/04; A61P 3/10; A61P 35/00; B01J 23/50; C07K 1/1075; C07K 14/575; A61K 38/00

(57) ABSTRACT

An improved method for the chemical synthesis of glycosylated peptides has been developed. The method uses stereoselective glycan synthesis and chemical peptide ligation to produce glycopeptides at lower-cost and with higher efficiency/yield compared to conventional methods. In particular, a method for the large-scale, chemical synthesis of hydroxylated amino acid building blocks and glycosylated amino acids suitable for solid phase peptide synthesis (SPPS) are disclosed. SPPS-based methods using the glycosylated amino acids to chemically synthesize adiponectin-like peptides are also described. In vivo studies show that the adiponectin mimicking glycopeptides exhibit significant anticancer, anti-obesity and insulin sensitizing effects. Pharmaceutical compositions of the synthetic glycopeptides and methods of use thereof in treating diseases associated with deficient adiponectin are also described.

### FIELD OF THE INVENTION

The invention is generally directed to glycopeptide synthesis, and in particular, methods of making and using glycosylated adiponectin peptides.

### BACKGROUND OF THE INVENTION

Adiponectin is a circulating glycoprotein mainly produced from adipocytes¹⁻⁴. It is a key regulator of glucose and lipid metabolism in peripheral organs such as skeletal muscle and liver, increasing systemic insulin sensitivity and energy homeostasis⁵⁻⁷. A decrease in the production of adiponectin is involved in the development of insulin resistance, type 2 diabetes, steatohepatitis, cardiovascular diseases and certain types of cancers⁸⁻¹⁰. Thus, it is believed that adiponectin supplementation may have therapeutic potential for metabolic, cancer, and cardiovascular diseases. Nevertheless, adiponectin-based therapeutics are not currently available, mainly due to the difficulty in obtaining the full-length, glycosylated human adiponectin¹¹.

Full-length adiponectin produced from expression in bacteria has been shown to be biologically inactive, due to the lack of glycosylation^{15,20}. The globular domain forms trimers exclusively and is less active than the full-length adiponectin, especially in its insulin-sensitizing and hepatoprotective functions²¹⁻²². The mammalian adiponectin collagenous domain contains four 5-(2S,5R)-hydroxylysine residues (at positions 65, 68, 77, 101) which are glycosylated with a glucosyl-galactose disaccharide¹²⁻¹³. The glycan structure is very different from the common O-linked or N-linked glycoproteins, and thus represents a huge challenge to covert the full-length human adiponectin protein or the glycosylated domain into a viable drug via recombinant approaches. This limitation hampers the detailed study of the glycosylated domain's function. In addition, two-dimensional gels showed multiple adiponectin isoforms²³⁻²⁵, which likely result from heterogeneous glycoforms. However, no information is available on their relative distribution.

Although efforts have been made to identify a minimal structure capable of eliciting the required pharmacological agonist activities, the structure-activity relationships of the individual domains within adiponectin have not been well defined¹⁴⁻¹⁹.

Thus, there is an urgent need for methods for producing glycosylated peptides, such as adiponectin. Synthetic adiponectin-based glycopeptides are urgently needed for treatment of metabolic, cancer, and cardiovascular diseases.

It is an object of the present invention to provide methods of making adiponectin peptides.

It is also an object of the present invention to provide methods for chemical synthesis of glycosylated adiponectin peptides.

It is a further object of the present invention to provide methods of synthesizing biologically active adiponectin peptides.

It is a further object of the present invention to provide more efficient methods of synthesizing glycosylated adiponectin peptides in high yield.

It is also an object of the present invention to provide compositions of glycosylated adiponectin peptides.

It is still an object of the present invention to provide methods for treating metabolic, cancer, and cardiovascular diseases in a subject in need thereof.

Any discussion of documents, acts, materials, devices, articles or the like which has been included in the present specification is not to be taken as an admission that any or all of these matters form part of the prior art base or were common general knowledge in the field relevant to the present disclosure as it existed before the priority date of each claim of this application.

### SUMMARY OF THE INVENTION

An improved method for the chemical synthesis of glycosylated peptides has been developed. The method uses stereoselective glycan synthesis and chemical peptide ligation to produce peptides including glycosylated amino acids at lower-cost and higher efficiency/yield compared to conventional methods. The method is useful for the preparation of glycosylated adiponectin-based peptides.

Methods for preparing amino acid building blocks are disclosed. In some forms, the amino acid building blocks prepared by the disclosed methods can have the structure of Formula (I):

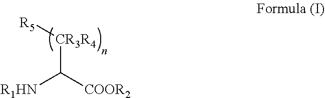

where n is an integer from 0 to 4; where each of R₁ and R₂ is independently a protecting group; where each occurrence of R₃ and R₄ is independently a hydrogen, a substituted alkyl, an unsubstituted alkyl, a hydroxyl, a thiol, or NR₁₃R₁₄, each of R₁₃ and R₁₄ is hydrogen, a substituted alkyl, or an unsubstituted alkyl; where R₅ is absent, a ketimine, an imidazole, an amino group, a carboxyl, a carboxylate, a hydroxyl, an amide, a thiol, a substituted alkyl, an unsubstituted alkyl, a sulfide, a substituted aryl, an unsubstituted aryl, or an indolizine, or R₅ is absent and (CR₃R₄)n is a substituted or unsubstituted alkyl that forms a ring with the nitrogen attached to C2 position, and when R₅ is or contains an amino group, a thiol group, and/or a hydroxyl group, the amino group, thiol group, and/or hydroxyl group is protected by a protecting group.

In some forms, each occurrence of the protecting group of Formula (I) can be independently acetyl, benzoyl, benzyl (“Bn”), methyl benzyl, β-methoxyethoxymethyl ether, dimethoxytrityl, methoxymethyl ether, methoxytrityl[(4-methoxyphenyl)diphenylmethyl], p-methoxybenzyl ether, p-methoxyphenyl ether, methylthiomethyl ether, pivaloyl, tetrahydropyranyl, tetrahydrofuran, trityl, silyl ether, methyl ether, ethoxyethyl ether, carbobenzyloxy, p-methoxybenzyl carbonyl, tert-butyloxycarbonyl (“Boc”), 9-fluorenylmethyloxycarbonyl (“Fmoc”), carbamate, p-methoxybenzyl, 3,4-dimethoxybenzyl, p-methoxyphenyl, tosyl, trichloroethyl chloroformate, nosyl, nps, methyl ester, benzyl ester, tert-butyl ester, 2,6-disubstituted phenol ester, silyl ester, orthoester, or oxazoline.

In some forms, the amino acid building blocks prepared by the disclosed methods can be optically pure, such as having the structure of Formula (XVI):

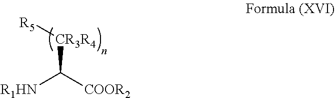

where n is an integer from 1 to 4; where each of R₁ and R₂ is independently a protecting group; where each occurrence of R₃ and R₄ is independently a hydrogen, a substituted alkyl, an unsubstituted alkyl, a hydroxyl, a thiol, or NR₁₃R₁₄, each of R₁₃ and R₁₄ is hydrogen, a substituted alkyl, or an unsubstituted alkyl; where R₅ is a ketimine, an imidazole, an amino group, a carboxyl, a carboxylate, a hydroxyl, an amide, a thiol, a substituted alkyl, an unsubstituted alkyl, a sulfide, a substituted aryl, an unsubstituted aryl, or an indolizine, and when R₅ is or contains an amino group, a thiol group, and/or a hydroxyl group, the amino group, thiol group, and/or hydroxyl group is protected by a protecting group; and where each occurrence of the protecting group is independently acetyl, benzoyl, benzyl, methyl benzyl, tert-butyloxycarbonyl, 9-fluorenylmethyloxycarbonyl, carbamate, carbobenzyloxy, p-methoxybenzyl, 3,4-dimethoxybenzyl, or p-methoxyphenyl.

In some forms, the amino acid building blocks prepared by the disclosed methods is (2S,5R)-hydroxylysine building blocks.

In some forms, methods for preparing an amino acid building block can include: (i) performing a reaction between a first reactant of Formula (II) and a second reactant of Formula (III):

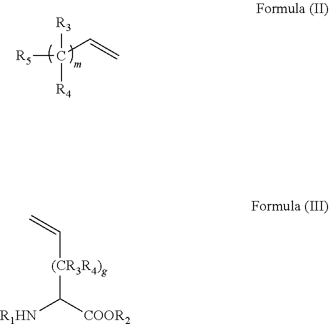

where each of m and g is an integer between 0 and 2 and R₁-R₅ are as defined above.

In some forms of the reaction in step (i), the first reactant can be optically pure and have the structure of Formula (IV):

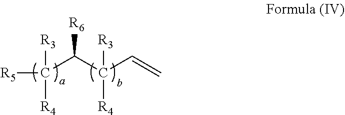

where each of a and b is independently 0 or 1; where each occurrence of R₃ and R₄ is independently a hydrogen, a substituted alkyl, an unsubstituted alkyl, a hydroxyl, a thiol, or NR₁₃R₁₄, each of R₁₃ and R₁₄ is hydrogen, a substituted alkyl, or an unsubstituted alkyl; where R₅ is a ketimine, an imidazole, an amino group, a carboxyl, a carboxylate, a hydroxyl, an amide, a thiol, a substituted alkyl, an unsubstituted alkyl, a sulfide, a substituted aryl, an unsubstituted aryl, or an indolizine, and when R₅ is or contains an amino group, a thiol group, and/or a hydroxyl group, the amino group, thiol group, and/or hydroxyl group is protected by any one of the protecting groups described above, such as acetyl, benzoyl, benzyl, methyl benzyl, tert-butyloxycarbonyl, 9-fluorenylmethyloxycarbonyl, carbamate, carbobenzyloxy, p-methoxybenzyl, 3,4-dimethoxybenzyl, and p-methoxyphenyl; and where R₆ is a substituted alkyl, an unsubstituted alkyl, a hydroxyl, a thiol, or NR₁₃R₁₄, each of R₁₃ and R₁₄ is hydrogen, substituted alkyl, or unsubstituted alkyl.

In some forms of the reaction in step (i), the second reactant can be optically pure and have the structure of Formula (IX):

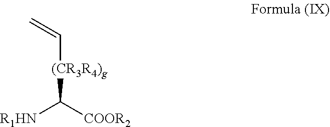

where g is an integer from 0 to 2; where each of R₁ and R₂ is independently a protecting group, such as acetyl, benzoyl, benzyl, methyl benzyl, tert-butyloxycarbonyl, 9-fluorenylmethyloxycarbonyl, carbamate, carbobenzyloxy, p-methoxybenzyl, 3,4-dimethoxybenzyl, or p-methoxyphenyl; and where each occurrence of R₃ and R₄ is independently a hydrogen, a substituted alkyl, an unsubstituted alkyl, a hydroxyl, a thiol, or NR₁₃R₁₄, each of R₁₃ and R₁₄ is hydrogen, a substituted alkyl, or an unsubstituted alkyl.

In some forms, the amino acid building blocks produced by the disclosed method contains two or more different protecting groups, such as two or three different protecting groups. Typically, each of the protecting groups contained in the amino acid building blocks is compatible with solid-phase peptide synthesis.

The method described herein can produce amino acid building blocks with a yield of at least 50%, at least 55%, at least 60%, at least 65%, at least 70%, or at least 75%.

Methods for preparing glycosylated amino acids are disclosed. In some forms, the glycosylated amino acids prepared by the disclosed methods can have the structure of Formula (XII):

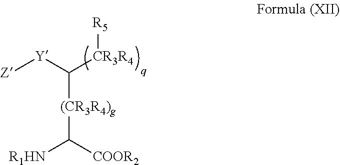

where each of p and q is an integer from 0 to 2; where Z′ is a monosaccharide moiety, a disaccharide moiety, an oligosaccharide moiety, or a polysaccharide moiety; where Y′ is an oxygen, a sulfur, or NR₁₅, R₁₅ is hydrogen, a substituted alkyl, or an unsubstituted alkyl; where each of R₁ and R₂ is independently a hydrogen or a protecting group; where each occurrence of R₃ and R₄ is independently a hydrogen, a substituted alkyl, an unsubstituted alkyl, a hydroxyl, a thiol, or NR₁₃R₁₄, each of R₁₃ and R₁₄ is hydrogen, a substituted alkyl, or an unsubstituted alkyl; where R₅ is absent, a ketimine, an imidazole, an amino group, a carboxyl, a carboxylate, a hydroxyl, an amide, a thiol, a substituted alkyl, an unsubstituted alkyl, a sulfide, a substituted aryl, an unsubstituted aryl, or an indolizine, and when R₅ is or contains an amino group, a thiol group, and/or a hydroxyl group, the amino group, thiol group, and/or hydroxyl group is protected by a protecting group; wherein each occurrence of the protecting group is independently acetyl, benzoyl, benzyl, methyl benzyl, β-methoxyethoxymethyl ether, dimethoxytrityl, methoxymethyl ether, methoxytrityl[(4-methoxyphenyl)diphenylmethyl], p-methoxybenzyl ether, p-methoxyphenyl ether, methylthiomethyl ether, pivaloyl, tetrahydropyranyl, tetrahydrofuran, trityl, silyl ether, methyl ether, ethoxyethyl ether, carbobenzyloxy, p-methoxybenzyl carbonyl, tert-butyloxycarbonyl, 9-fluorenylmethyloxycarbonyl, carbamate, p-methoxybenzyl, 3,4-dimethoxybenzyl, p-methoxyphenyl, tosyl, trichloroethyl chloroformate, nosyl, nps, methyl ester, benzyl ester, tert-butyl ester, 2,6-disubstituted phenol ester, silyl ester, orthoester, or oxazoline,

In some forms, the glycosylated amino acids prepared by the disclosed methods can have the structure of Formula (XXII) or Formula (XXIII):

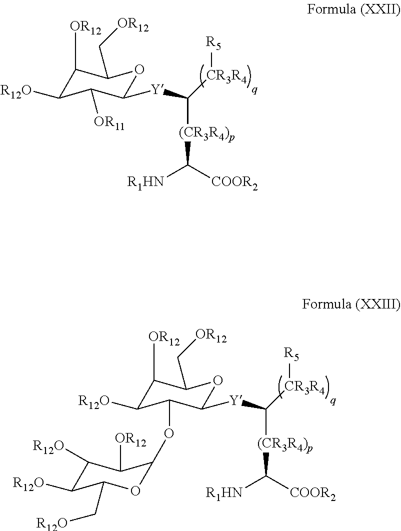

where each of R₁, R₂, R₁₁ and R₁₂ can be independently a hydrogen or a protecting group as described above, such as acetyl, benzoyl, benzyl, methyl benzyl, tert-butyloxycarbonyl, 9-fluorenylmethyloxycarbonyl, carbamate, carbobenzyloxy, p-methoxybenzyl, 3,4-dimethoxybenzyl, and p-methoxyphenyl; where R₃-R₅ and Y′ are as described above for Formula (XII).

In some forms, the glycosylated amino acids prepared by the disclosed methods are glycosylated (2S,5R)-hydroxylysine.

In some forms, the glycosylated amino acids produced by the disclosed method contains two or more different protecting groups, such as two or three different protecting groups. Typically, each of the protecting groups contained in the resulting glycosylated amino acids is compatible with solid-phase peptide synthesis.

In some forms, methods for preparing an amino acid building block can include: (ii) performing a reaction between a saccharide of Formula (XIII) and an amino acid building block of Formula (I):

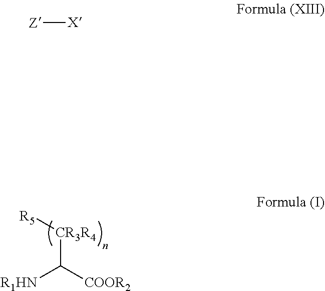

where Z′ and R₁-R₅ are as defined above; where n is an integer from 0 to 4; and wherein X′ is a leaving group.

In some forms of the reaction in step (ii), the saccharide can have the structure of Formula (XIV) or Formula (XV):

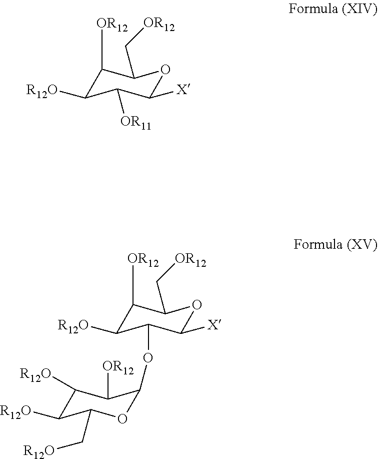

where X′ is a leaving group and where each occurrence of R₁₁ and R₁₂ is independently a hydrogen or a protecting group, such as acetyl, benzoyl, benzyl, methyl benzyl, tert-butyloxycarbonyl, 9-fluorenylmethyloxycarbonyl, carbamate, carbobenzyloxy, p-methoxybenzyl, 3,4-dimethoxybenzyl, or p-methoxyphenyl, such as methyl benzyl (e.g. 4-methyl benzyl) or acetyl.

The method described above for preparing glycosylated amino acids can be performed in a suitable solvent, such as a mixture of dimethylformamide and dichloromethane.

The method described above can produce glycosylated amino acids with a yield of at least 30%, at least 35%, at least 40%, at least 45%, or at least 50%.

In some forms of the reactions performed in step (i) and/or step (ii), a catalyst is used. Typically, the catalyst used in the reaction performed in step (i) and/or step (ii) is non-toxic. In some forms, the methods for preparing the amino acid building blocks and/or glycosylated amino acids does not include HPLC separation/purification.

In some forms, the amino acid building block used in step (ii) can be prepared by the reaction in step (i).

Methods for the synthesis of glycopeptides are also disclosed. In some forms, a method for chemical synthesis of a glycopeptide involves performing solid phase peptide synthesis (SPPS) to assemble a peptide having a desired amino acid sequence and incorporating one or more glycosylated amino acids of Formula (XII) into the peptide in one or more desired positions. Preferably, the one or more glycosylated amino acids are produced by the method for producing glycosylated amino acids of Formula (XII) described above and elsewhere in this disclosure. In some forms, Fmoc-based solid phase peptide synthesis is used.

In some forms, synthesis of glycopeptides can further include ligating two or more peptide fragments to form the glycopeptide. The two or more peptide fragments (e.g., at least one of which can be chemically synthesized by the disclosed SPPS) can be ligated using salicylaldehyde ester-mediated ligation at serine and/or threonine residues.

Glycopeptides chemically synthesized by the disclosed methods and compositions thereof are also provided. In some forms, the glycopeptide includes the amino acid sequence of any one of SEQ ID NOs:1-3 or a sequence having at least 75% sequence identity thereto. In some forms, the glycopeptide includes one or more domains of human adiponectin or portions thereof. For example, in some forms, the glycopeptide contains the amino acid sequence of SEQ ID NO:3 which has a portion of the variable region and the collagenous domain of human adiponectin.

In particular forms, an isolated glycopeptide includes or consists of the collagenous domain of human adiponectin or a portion thereof. Preferably, the glycopeptide includes one or more hydroxylysine residues in the collagenous domain, and one or more glycosylated lysine residues in the collagenous domain. In preferred forms, the one or more glycosylated lysine residues are chemically synthesized by the method for producing glycosylated amino acids of Formula (XII) described above. In preferred forms, the human adiponectin has the amino acid sequence of SEQ ID NO:1. An exemplary collagenous domain of human adiponectin is the amino acid sequence of SEQ ID NO:2.

Preferably, the one or more hydroxylysine residues are (2S,5R)-hydroxylysine (e.g., 5-(2S,5R)-hydroxylysine). The one or more hydroxylysine residues and/or the one or more glycosylated lysine residues can be selected from lysine residues 65, 68, 77, and 101 of human adiponectin. In some forms, the same residue(s) is hydroxylated and glycosylated.

In some forms, the glycopeptide is glycosylated at two or more lysine residues (e.g., lysine residues 65, 68, 77, and 101 of human adiponectin), such as lysine residues 68 and 77. In some forms, the glycopeptide is glycosylated at three or more lysine residues. In some forms, the glycopeptide is glycosylated at all four of lysine residues 65, 68, 77, and 101.

Exemplary sugar moieties with which the residues can be glycosylated include, without limitation, a glucosylgalactosyl moiety, a glucosylglucosyl moiety, a galactosylglucosyl moiety, or a galactosylgalactosyl moiety. In preferred forms, the one or more glycosylated lysine residues are glycosylated with a glucosylgalactosyl moiety. In some preferred forms, the one or more glycosylated lysine residues are glycosylated with 2-O-α-D-glucopyranosyl-D-galactose.

The glycopeptide may exert certain effects when contacted with cells or tissue in vitro or in vivo. For example, in some forms, administration of the glycopeptide to a subject reduces cancer cell proliferation, viability, or metastasis, reduces tumor growth or tumor burden, reduces body weight or body fat mass, improves glucose tolerance, improves insulin sensitivity, reduces or inhibits gluconeogenesis, reduces triglyceride or cholesterol levels (e.g., in the liver or serum), reduces or inhibits inflammation (e.g., in the liver), reduces the expression levels of one or more liver injury biomarkers, or combinations, improving immune cell development and function thereof. Non-limiting examples of liver injury biomarkers include ALT, AST, TNFα, CCL2, LDLR, COL1, COL6, bilirubin (TBL), alkaline phosphatase (ALP), Interleukin-6 (IL-6), and Interleukin-10 (IL-10).

In some forms, the subject administered with the glycopeptide suffers from obesity, cancer, steatohepatitis or other liver disease, a metabolic disease, Type 1 diabetes, Type 2 diabetes, obesity, metabolic syndrome, hypertension, atherosclerosis, inflammation, hyperglycemia, endothelial dysfunction, insulin resistance, or a combination thereof.

Also described are pharmaceutical compositions including the disclosed glycopeptide and a pharmaceutically acceptable carrier. The pharmaceutical composition can include a plurality of copies of the glycopeptide. In some forms, the pharmaceutical composition includes two or more isoforms of the glycopeptide (glycoforms).

Methods of using the glycopeptides and compositions thereof are provided. In preferred forms, the compositions are used therapeutically. For example, disclosed is a method of treating a subject having a disease, disorder, or condition by administering to the subject an effective amount of a disclosed glycopeptide containing pharmaceutical composition.

The disease, disorder, or condition can be associated with reduced or low adiponectin levels, for example, in the serum. In some forms, the disease, disorder, or condition is hypoadiponectinemia. In some forms, the disease, disorder, or condition is cancer, steatohepatitis or other liver disease, Type 1 diabetes, Type 2 diabetes, obesity, metabolic syndrome, hypertension, atherosclerosis, inflammation, hyperglycemia, endothelial dysfunction, or insulin resistance.

Preferably, the subject treated in accordance with the disclosed methods is human.

Additional advantages of the disclosed methods will be set forth in part in the description which follows, and in part will be understood from the description, or can be learned by practice of the disclosed methods and compositions. The advantages of the disclosed method and compositions will be realized and attained by means of the elements and combinations particularly pointed out in the appended claims. It is to be understood that both the foregoing general description and the following detailed description are exemplary and explanatory only and are not restrictive of the invention as claimed.

### BRIEF DESCRIPTION OF THE DRAWINGS

The accompanying drawings, which are incorporated in and constitute a part of this specification, illustrate several embodiments of the disclosed methods and compositions and together with the description, serve to explain the principles of the disclosed method and compositions.

FIG. 1 is a schematic showing the primary structure (amino acid sequence) of human adiponectin (SEQ ID NO:1) with detailed amino acid sequences of the collagenous domain.

FIG. 2A is a scheme showing the synthesis of glycopeptide hAdn-WM77. The conditions and reagents used as follows: (a) CH₃CN/H₂O/diethylamine (4.5/4.5/1, v/v/), r.t., 2 h, lyophilization; (b) hAdn-WM-a, pyridine/acetic acid (1/1, mole/mole), r.t., 4 h. FIG. 2B is a graph showing the UPLC-MS trace of purified hAdn-WM77. FIG. 2C is a graph showing the ESI-MS spectrum of purified hAdn-WM77. ESI-MS calcd. for C₃₀₂H₄₇₀N₉₂O₁₀₆S, [M+4H]4+m/z=1780.4, found 1780.4; [M+5H]5+m/z=1424.5, found 1424.6; [M+6H]6+m/z=1187.3, found 1187.3; [M+7H]7+m/z=1017.8, found 1017.7; [M+8H]8+m/z=890.7, found 890.8; [M+9H]9+m/z=791.9, found 792.0; [M+10H]10+m/z=712.8, found 712.6; [M+11H]11+m/z=648.1, found 648.4. FIG. 2D is a schematic depiction of the various glyACD glycoforms, indicating the composition of R1-R4 for the indicated peptide having the sequence of SEQ ID NO:3. FIGS. 2E-2F are schematics showing the synthesis of glycopeptide hAdn-WM65 via the synthesis and subsequent ligation of two precursor peptide fragments, namely WM65-b (FIG. 2E) and WM-a (FIG. 2F). The sequence identifier numbers for the amino acid sequences of the peptides depicted in FIGS. 2A-2F are Adn-WM77-b: SEQ ID NO:4, Adn-WM77-b1: SEQ ID NO:4, Adn-WM-a: SEQ ID NO:5, Adn-WM77: SEQ ID NO:3, WM65-a: SEQ ID NO:4, WM65-b: SEQ ID NO:4, WM65-b1: SEQ ID NO:4, WM-a: SEQ ID NO:5, WM65*: SEQ ID NO:3, and WM65: SEQ ID NO:3.

FIGS. 3A-3B are bar graphs showing the Eₘₐₓ (FIG. 3A) and EC₅₀ (FIG. 3B) of adiponectin or peptides with no glycans (hAdn-WM), or mono-, di-, tri- and tetra-glycans. The glyACD and adiponectin elicit synergistic anti-proliferative activity in human MDA-MB-231 cells. After serum starvation for 24 hours, MDA-MB-231 cells were treated with different concentrations of hAdn-WM, glyACD, full-length human adiponectin, or their combinations. After incubation for another 24 hours, cells were harvested for manual counting. FIG. 3C is a graph showing the inhibition rate on MDA-MB-231 cells of the indicated peptides. MDA-MB-231 cells were treated with different concentrations of adiponectin in the absence or presence of low (2.5 μg/ml) or high (20 μg/ml) doses of hAdn-WM or hAdn-WM656877101. FIG. 3D is a heatmap showing the percentage inhibition on cell growth. MDA-MB-231 cells were treated with 0, 0.22, 0.33, 0.49, 0.74, 1.11, 1.67, 2.5 μg/ml adiponectin in combination with 0, 0.3125, 0.625, 1.25, 2.5, 5, 10, 20 μg/ml hAdn-WM or hAdn-WM656877101. The percentage inhibition on cell growth was calculated and indicated by different intensity of the blue color. Data are presented as mean±SEM (n=6). *, P<0.05 vs full-length human adiponectin; ♦, P<0.05 vs peptide with no glycans (hAdn-WM); ▴, P<0.05 vs peptide with tetra-glycans (hAdn-WM656877101).

FIGS. 4A-4B are graphs showing the tumor volume (FIG. 4A) and body weight and tumor/body weight percentage (FIG. 4B) after human breast cancer MDA-MB-231 cells were incubated with phosphate buffer saline (PBS) or 20 μg/ml hAdn-WM6877 and injected into nude mice. FIGS. 4C-4D are graphs showing the tumor volume (FIG. 4C) and body weight and tumor/body weight percentage (FIG. 4D) after human breast cancer MDA-MB-231 cells were incubated with phosphate buffer saline (PBS) or 20 μg/ml hAdn-WM6877 and injected into NOD/Scid mice. FIGS. 4E-4F are graphs showing the tumor volume (FIG. 4E) and body weight and tumor/body weight percentage (FIG. 4F) when PyVT-AKO mice were intraperitoneally injected with PBS or hAdn-WM6877. Data are presented as mean±SEM (n=6); *, P<0.05 vs corresponding vehicle controls. The significant differences between groups were analyzed by t-test or two-way ANOVA.

FIGS. 5A-5D are graphs showing body weight (FIG. 5A), fat mass composition (FIG. 5B), glucose tolerance (FIG. 5C), and insulin tolerance (FIG. 5D) of adiponectin deficient (AKO) mice intraperitoneally injected with PBS or hAdn-WM6877 (40 g/mouse/day) over four weeks. Results are presented as fold changes against the starting points (before injection). In FIGS. 5C-5D, intraperitoneal glucose and insulin tolerance tests were performed after four-weeks of treatment. FIGS. 5E-5G are graphs showing oxygen consumption (VO₂; FIG. 5E), carbon dioxide production (VCO₂; FIG. 5F), and respiratory exchange ratio (RER; FIG. 5G) in mice treated with PBS or hAdn-WM6877 assessed via indirect calorimetry. FIGS. 5H-5K are graphs showing fasting glucose levels (FIG. 5H), fasting insulin levels (FIG. 5I), triglyceride levels (FIG. 5J), and cholesterol levels (FIG. 5K) in mice treated with PBS or hAdn-WM6877. At the end of treatment, mice were sacrificed after fasting with food removal for 16 hours. Serum was collected to measure circulating levels of glucose, insulin, triglyceride and cholesterol. Data are presented as mean±SEM (n=6); *, P<0.05 vs corresponding vehicle controls. Differences between means were determined using Two-Way ANOVA with Fisher's LSD test. Significant differences between groups were analyzed by t-test or two-way ANOVA.

FIGS. 6A-6I show that the hAdn-WM6877 glyACD alleviates HFD-induced fatty liver injuries. The adiponectin deficient (AKO) mice of the C57BL/6J background were fed with a high fat diet (HFD) starting from the age of four weeks. After eight-weeks of HFD, equal volume of PBS or hAdn-WM6877 (40 μg/mouse/day) was intraperitoneally injected into AKO mice for another four weeks. At the end of treatment, mice were sacrificed after fasting with food removal for 16 hours. FIGS. 6A-6D are graphs showing triglyceride contents in liver samples (FIG. 6A), ALT in blood circulation (FIG. 6B), cholesterol contents in liver samples (FIG. 6C), and AST in blood circulation (FIG. 6D). FIGS. 6E-6I are graphs showing quantitative PCR (QPCR) based gene expression levels of TNFA (FIG. 6E), CCL2 (FIG. 6F), LDLR (FIG. 6G), COL1 (FIG. 6H), and COL6 (FIG. 6I) in liver tissue. Data are presented as mean±SEM (n=6); *, P<0.05 vs corresponding vehicle controls (n=6). Differences between means were determined using the unpaired Student's t-tests.

### DETAILED DESCRIPTION OF THE INVENTION

The disclosed methods and compositions can be understood more readily by reference to the following detailed description of particular embodiments and the Examples included therein and to the Figures and their previous and following description.

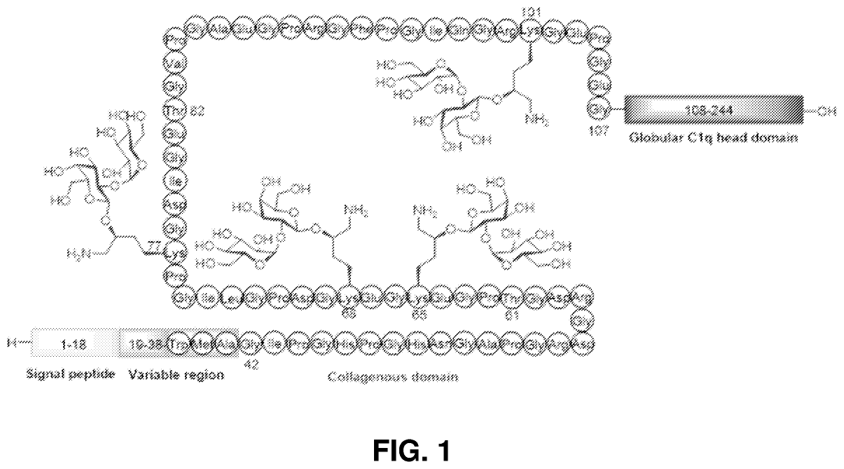

Adiponectin is a hormone secreted abundantly from adipose tissue, and is an insulin-sensitizing adipokine with antidiabetic, anti-atherogenic, anti-inflammatory and cardioprotective properties. Its human form is composed of 244 amino acid residues divided into four structurally distinct domains: a signal peptide, a variable region, a collagenous domain, and a globular domain that binds to the adiponectin receptors (see FIG. 1). In circulation, adiponectin exists mainly in three isoforms including trimeric, hexameric and high-molecular-weight (HMW, at least 18 protomers) oligomers and its monomeric form has not been detected under native conditions. This oligomerization represents a key mechanism to regulate the biological activities of adiponectin, and the HMW form is considered the most active form. Studies show that post-translational modification, especially glycosylation, plays a key role in HMW formation and its bioactivity.

Since the discovery of adiponectin and its role as an insulin-sensitizer and an inhibitory factor for cancer development, large scale production of adiponectin or its mimetics has been desirable. Currently, full adiponectin is mainly produced in mammalian cell lines, which is very costly. However, traditional large-scale bio-fermentation techniques fail to fully reproduce mammalian post-translational modifications, resulting in a failure to form bioactive HMW adiponectin oligomers. Furthermore, it is very hard to produce sufficient amounts of adiponectin from eukaryotic cell cultures, while adiponectin produced in prokaryotic cells does not have biological activity. Hence, development of full-length adiponectin as a therapeutic is unrealized. Therefore, development of adiponectin mimetics including synthesized peptides, mimetic proteins and chemicals, provides alternative approaches, which are low-cost and show high modification capacity. However, artificially mimicking post-translational modifications (for example, glycosylation, phosphorylation) on synthesized adiponectin is difficult. Traditional methods are expensive and time consuming.

A new method for the chemical synthesis of adiponectin which overcomes these challenges has been developed. The method is low-cost and highly efficient compared with traditional methods and facilitates the production of glycosylated peptides. As shown in the working Examples, all possible fifteen glycoforms of homogenously glycosylated, 69-amino acid, adiponectin collagenous domains (ACD) have been chemically and individually synthesized using stereoselective glycan synthesis and chemical peptide ligation.

Biological and pharmacological studies were used to assess the relationship between glycan pattern and function in the inhibition of cancer cell growth and regulation of systemic energy metabolism, as well as whether glycosylated ACD peptides could elicit the beneficial effects of full-length adiponectin. The glycopeptides demonstrated significant anticancer, anti-obesity and insulin sensitizing effects. In particular, hAdn-WM6877 was tested in detail using different mouse models and it exhibited in vivo anti-tumor, insulin sensitizing, and hepatoprotective activities.

Thus, the in vitro and in vivo tests showed positive effects of the synthesized glycopeptides on cancer development and metabolic function. The studies demonstrate the possibility of using synthetic glycopeptides, which are shorter than the full-length adiponectin, as adiponectin mimics for the development of novel therapeutics to treat diseases associated with deficient adiponectin. It is also contemplated that the disclosed platforms and methodology allow for synthesis of full-length, biologically active glycosylated proteins, including adiponectin.

Additional advantages of the disclosed methods and compositions will be set forth in part in the description which follows, and in part will be understood from the description, or can be learned by practice of the disclosed method and compositions. The advantages of the disclosed method and compositions will be realized and attained by means of the elements and combinations particularly pointed out in the appended claims. It is to be understood that both the foregoing general description and the following detailed description are exemplary and explanatory only and are not restrictive of the invention as claimed.

It is to be understood that the disclosed method and compositions are not limited to specific synthetic methods, specific analytical techniques, or to particular reagents unless otherwise specified, and, as such, can vary. It is also to be understood that the terminology used herein is for the purpose of describing particular embodiments only and is not intended to be limiting.

### I. Definitions

“Sensitive” refers to the presence of an intended response to an agent in a subject. The agent can be a physiological molecule such as a hormone or a drug that is administered to the subject for a specific purpose. Conversely, “insensitive” refers to the lack of an intended response in a subject to the agent. The response may be on a cellular or physiological level. For example, a subject who is administered a drug to induce weight loss may be characterized as “insensitive” to the drug if they do not exhibit weight loss upon administration of dosage regimen that is otherwise effective in the wider population. Such “insensitive” subjects may be referred to as nonresponsive, poorly responsive, non-susceptible, or less susceptible.

As used herein, the term “subject” means any individual, organism or entity. The subject can be a vertebrate, for example, a mammal (e.g., rat, rabbit, mouse, dog, cat, goat, pig, or horse). Thus, the subject can be a human. The term does not denote a particular age or sex. Thus, adult and newborn subjects, whether male or female, are intended to be covered. The subject may be healthy or suffering from or susceptible to a disease, disorder or condition. A “patient” refers to a subject afflicted with a disease or disorder. The term “patient” includes human and veterinary subjects.

“Obese,” as used herein, refers to a subject having a body mass index of 30 kg/m² or more. “Overweight” and “pre-Obese,” as used herein, refer to subject having a body mass index of between 25 kg/m² and 30 kg/m².

“Tumor burden” as used herein, refers to the number of cancer cells, the size or mass of a tumor, or the total amount of tumor/cancer in a particular region of a subject. Methods of determining tumor burden for different contexts are known in the art, and the appropriate method can be selected by the skilled person. For example, in some forms, tumor burden may be assessed using guidelines provided in the Response Evaluation Criteria in Solid Tumors (RECIST).

“Analog” and “derivative,” are used herein interchangeably, and refer to a compound that possesses the same core as a parent compound, but differs from the parent compound in bond order, in the absence or presence of one or more atoms and/or groups of atoms, and combinations thereof. The derivative can differ from the parent compound, for example, in one or more substituents present on the core, which may include one or more atoms, functional groups, or substructures. The derivative can also differ from the parent compound in the bond order between atoms within the core. In general, a derivative can be imagined to be formed, at least theoretically, from the parent compound via chemical and/or physical processes.

A “biomarker” refers generally to a molecule, including without limitation a gene or product thereof, nucleic acids (e.g., DNA, RNA), protein/peptide/polypeptide, carbohydrate structure, lipid, glycolipid, which can be detected in a tissue or cell to provide information that is predictive, diagnostic, and/or prognostic. In some forms, the biomarker can include, without limitation, a gene or gene product (e.g., a protein which may be expressed on the surface of or within a cell or tissue), chromosomal aberrations, genomic amplifications or copy number variations, and physical cellular structures. In some forms, a biomarker whose expression levels can be predictive of liver injury or permits the detection or diagnosis of liver injury is referred to as a “liver injury biomarker.”

As used herein in reference to the peptides, the term “isolated” means a peptide that is in a form that is relatively free from material such as contaminating polypeptides, lipids, nucleic acids and other cellular material that can normally be associated with the peptide in a cell or that is associated with the peptide in a library or in a crude preparation. A purified peptide can yield a single major band on a non-reducing polyacrylamide gel. A purified peptide can be at least about 75% pure (e.g., at least 80%, 85%, 90%, 95%, 97%, 98%, 99%, or 100% pure). Purified peptides can be obtained by, for example, extraction from a natural source, by chemical synthesis, or by recombinant production in a host cell or transgenic plant, and can be purified using, for example, affinity chromatography, immunoprecipitation, size exclusion chromatography, and ion exchange chromatography.

By “contact,” “contacting” or “exposing” is meant to allow or promote a state of immediate proximity or physical association between at least two elements. For example, to expose a peptide to a cell is to provide contact between the cell and the peptide. The term encompasses, but is not limited to, penetration of the contacted peptide to the interior of the cell by any suitable means, e.g., via transfection, electroporation, transduction, nanoparticle delivery, etc.

By “pharmaceutically acceptable” is meant a material that can be administered to a subject without causing undesirable biological effects or interacting in a deleterious manner with any of the other components of the pharmaceutical composition in which it is contained.

As used herein, the term “carrier” or “excipient” refers to an organic or inorganic ingredient, natural or synthetic inactive ingredient in a formulation, with which one or more active ingredients are combined.

The term, “in vitro” refers to an artificial environment and to processes or reactions that occur within an artificial environment. In vitro environments include, but are not limited to, in solution or suspension, and cell cultures. The term “in vivo” refers to in or associated with an organism, such as an animal.

As used herein, “treatment” refers to the medical management of a patient with the intent to cure, ameliorate, stabilize, or prevent a disease, pathological condition, or disorder. This term includes active treatment, that is, treatment directed specifically toward the improvement of a disease, pathological condition, or disorder, and also includes causal treatment, that is, treatment directed toward removal of the cause of the associated disease, pathological condition, or disorder. In addition, this term includes palliative treatment, that is, treatment designed for the relief of symptoms rather than the curing of the disease, pathological condition, or disorder; preventative treatment, that is, treatment directed to minimizing or partially or completely inhibiting the development of the associated disease, pathological condition, or disorder; and supportive treatment, that is, treatment employed to supplement another specific therapy directed toward the improvement of the associated disease, pathological condition, or disorder.

As used herein, the term “prevent” or “preventing” means to administer a composition to a subject or a system at risk for or having a predisposition for one or more symptoms caused by a disease or disorder to cause cessation of a particular symptom of the disease or disorder, a reduction or prevention of one or more symptoms of the disease or disorder, a reduction in the severity of the disease or disorder, the complete ablation of the disease or disorder, or stabilization or delay of the development or progression of the disease or disorder.

The term “effective amount,” or “therapeutically effective amount” as used herein, refers to an amount of an agent that is sufficient to elicit a desired biological and/or a pharmacologic response. In some forms, an “effective amount” or “therapeutically effective amount” means a quantity sufficient to alleviate or ameliorate one or more symptoms of a disorder, disease, or condition being treated. As will be appreciated by the skilled artisan, the effective amount of an agent, e.g., a glycopeptide formulation, may vary depending on various factors, for example, the desired biological response, the site being targeted, and on the agent being used.

As used herein, the terms “reduce” and “inhibit” mean to decrease an activity, response, condition, disease, or other biological parameter. This can include, but is not limited to, the complete ablation of the activity, response, condition, or disease. It is understood that this is typically in relation to a standard or expected value. The reduction or inhibition may be a 10, 20, 30, 40, 50, 60, 70, 80, 90, 100%, or any amount of reduction in between as compared to native or control levels. In some forms, inhibition or reduction is relative to a state prior to administration of one or more therapeutics. In some forms, inhibition or reduction is relative to a control that is not administered one or more therapeutics.

The term “percent (%) sequence identity” describes the percentage of nucleotides or amino acids in a candidate sequence that are identical with the nucleotides or amino acids in a reference sequence, after aligning the sequences and introducing gaps, if necessary, to achieve the maximum percent sequence identity. Alignment for purposes of determining percent sequence identity can be achieved in various ways that are within the skill in the art, for instance, using publicly available computer software such as BLAST, BLAST-2, ALIGN, ALIGN-2 or Megalign (DNASTAR) software. Appropriate parameters for measuring alignment, including any algorithms needed to achieve maximal alignment over the full-length of the sequences being compared can be determined by known methods.

“Identity” can be readily calculated by known methods, including, but not limited to, those described in Computational Molecular Biology, Lesk, A. M., Ed., Oxford University Press, New York, 1988; Biocomputing: Informatics and Genome Projects, Smith, D. W., Ed., Academic Press, New York, 1993; Computer Analysis of Sequence Data, Part I, Griffin, A. M., and Griffin, H. G., Eds., Humana Press, New Jersey, 1994; Sequence Analysis in Molecular Biology, von Heinje, G., Academic Press, 1987; and Sequence Analysis Primer, Gribskov, M. and Devereux, J., Eds., M Stockton Press, New York, 1991; and Carillo, H., and Lipman, D., SIAM J Applied Math., 48: 1073 (1988). Preferred methods to determine identity are designed to give the largest match between the sequences tested. Methods to determine identity and similarity are codified in publicly available computer programs. The percent identity between two sequences can be determined by using analysis software (i.e., Sequence Analysis Software Package of the Genetics Computer Group, Madison Wis.) that incorporates the Needelman and Wunsch, (J. Mol. Biol., 48: 443-453, 1970) algorithm (e.g., NBLAST, and XBLAST). In some forms, the default parameters can be used to determine the identity for the polynucleotides or polypeptides of the present disclosure.

In some forms, the % sequence identity of a given nucleic acid or amino acid sequence C to, with, or against a given nucleic acid or amino acid sequence D (which can alternatively be phrased as a given sequence C that has or includes a certain % sequence identity to, with, or against a given sequence D) is calculated as follows:

100 times the fraction W/Z,

where W is the number of nucleotides or amino acids scored as identical matches by the sequence alignment program in that program's alignment of C and D, and where Z is the total number of nucleotides or amino acids in D. It will be appreciated that where the length of sequence C is not equal to the length of sequence D, the % sequence identity of C to D will not equal the % sequence identity of D to C.

“Substituted,” as used herein, refers to all permissible substituents of the compounds or functional groups described herein. In the broadest sense, the permissible substituents include acyclic and cyclic, branched and unbranched, carbocyclic and heterocyclic, aromatic and nonaromatic substituents of organic compounds. Illustrative substituents include, but are not limited to, halogens, hydroxyl groups, or any other organic groupings containing any number of carbon atoms, preferably 1-14 carbon atoms, and optionally include one or more heteroatoms such as oxygen, sulfur, or nitrogen grouping in linear, branched, or cyclic structural formats. Representative substituents include a substituted or unsubstituted alkyl, a substituted or unsubstituted alkenyl, a substituted or unsubstituted alkynyl, a substituted or unsubstituted heterocyclyl, a substituted or unsubstituted phenyl, a substituted or unsubstituted aryl, a substituted or unsubstituted heteroaryl, a substituted or unsubstituted polyaryl, a substituted or unsubstituted polyheteroaryl, a substituted or unsubstituted aralkyl, a halogen, a hydroxyl, an alkoxy, a phenoxy, an aroxy, a silyl, a thiol, an alkylthio, a substituted alkylthio, a phenylthio, an arylthio, a cyano, an isocyano, a nitro, a substituted or unsubstituted carbonyl, a carboxyl, an amino, an amido, an oxo, a sulfinyl, a sulfonyl, a sulfonic acid, a phosphonium, a phosphanyl, a phosphoryl, a phosphonyl, an amino acid, poly(lactic-co-glycolic acid), a peptide, a polypeptide group, and a sugar group (such as a glucose group, an acetylated glucose, a fructose, an acetylated fructose, etc.). Such a substituted or unsubstituted alkyl, a substituted or unsubstituted alkenyl, a substituted or unsubstituted alkynyl, a substituted or unsubstituted heterocyclyl, a substituted or unsubstituted phenyl, a substituted or unsubstituted aryl, a substituted or unsubstituted heteroaryl, a substituted or unsubstituted polyaryl, a substituted or unsubstituted polyheteroaryl, a substituted or unsubstituted aralkyl, a halogen, a hydroxyl, an alkoxy, a phenoxy, an aroxy, a silyl, a thiol, an alkylthio, a substituted alkylthio, a phenylthio, an arylthio, a cyano, an isocyano, a nitro, a substituted or unsubstituted carbonyl, a carboxyl, an amino, an amido, an oxo, a sulfinyl, a sulfonyl, a sulfonic acid, a phosphonium, a phosphanyl, a phosphoryl, a phosphonyl, an amino acid, poly(lactic-co-glycolic acid), a peptide, a polypeptide group, and a sugar group can be further substituted.

Heteroatoms such as nitrogen may have hydrogen substituents and/or any permissible substituents of organic compounds described herein which satisfy the valences of the heteroatoms. It is understood that “substitution” or “substituted” includes the implicit proviso that such substitution is in accordance with permitted valence of the substituted atom and the substituent, and that the substitution results in a stable compound, i.e. a compound that does not spontaneously undergo transformation such as by rearrangement, cyclization, elimination, etc.

“Alkyl,” as used herein, refers to the radical of saturated aliphatic groups, including straight-chain alkyl groups, branched-chain alkyl, and cycloalkyl (alicyclic). In some forms, a straight chain or branched chain alkyl has 30 or fewer carbon atoms in its backbone (e.g., C₁-C₃₀ for straight chains, C₃-C₃₀ for branched chains), 20 or fewer, 15 or fewer, or 10 or fewer. Alkyl includes methyl, ethyl, n-propyl, isopropyl, n-butyl, isobutyl, t-butyl, pentyl, hexyl, heptyl, octyl, decyl, tetradecyl, hexadecyl, eicosyl, tetracosyl and the like. Likewise, a cycloalkyl is a non-aromatic carbon-based ring composed of at least three carbon atoms, such as a nonaromatic monocyclic or nonaromatic polycyclic ring containing 3-30 carbon atoms, 3-20 carbon atoms, or 3-10 carbon atoms in their ring structure, and have 5, 6 or 7 carbons in the ring structure. Cycloalkyls containing a polycyclic ring system can have two or more non-aromatic rings in which two or more carbons are common to two adjoining rings (i.e., “fused cycloalkyl rings”). Examples of cycloalkyl groups include, but are not limited to, cyclopropyl, cyclobutyl, cyclopentyl, cyclohexyl, cycloheptyl, cyclooctanyl, etc.

The term “alkyl” as used throughout the specification, examples, and claims is intended to include both “unsubstituted alkyls” and “substituted alkyls,” the latter of which refers to alkyl moieties having one or more substituents replacing a hydrogen on one or more carbons of the hydrocarbon backbone. Such substituents can be any substituents described above, e.g., halogen (such as fluorine, chlorine, bromine, or iodine), hydroxyl, carbonyl (such as a carboxyl, alkoxycarbonyl, formyl, or an acyl), thiocarbonyl (such as a thioester, a thioacetate, or a thioformate), aryl, alkoxyl, aralkyl, phosphonium, phosphanyl, phosphonyl, phosphoryl, phosphate, phosphonate, a phosphinate, amino, amido, amidine, imine, cyano, nitro, azido, oxo, sulfhydryl, thiol, alkylthio, silyl, sulfinyl, sulfate, sulfonate, sulfamoyl, sulfonamido, sulfonyl, heterocyclyl, an aromatic or heteroaromatic moiety. —NRR′, wherein R and R′ are independently hydrogen, alkyl, or aryl, and wherein the nitrogen atom is optionally quaternized; —SR, wherein R is a phosphonyl, a sulfinyl, a silyl a hydrogen, an alkyl, or an aryl; —CN; —NO₂; —COOH; carboxylate; —COR, —COOR, or —CON(R)₂, wherein R is hydrogen, alkyl, or aryl; imino, silyl, ether, haloalkyl (such as —CF3, —CH₂—CF₃, —CCl₃); —CN; —NCOCOCH₂CH₂; —NCOCOCHCH; and —NCS; and combinations thereof. The term “alkyl” also includes “heteroalkyl.”

It will be understood by those skilled in the art that the moieties substituted on the hydrocarbon chain can themselves be substituted, if appropriate. For instance, the substituents of a substituted alkyl may include halogen, hydroxy, nitro, thiols, amino, aralkyl, azido, imino, amido, phosphonium, phosphanyl, phosphoryl (including phosphonate and phosphinate), oxo, sulfonyl (including sulfate, sulfonamido, sulfamoyl and sulfonate), and silyl groups, as well as ethers, alkylthios, carbonyls (including ketones, aldehydes, carboxylates, and esters), haloalkyls, —CN and the like. Cycloalkyls can be substituted in the same manner.

Unless the number of carbons is otherwise specified, “lower alkyl” as used herein means an alkyl group, as defined above, but having from one to ten carbons, more preferably from one to six carbon atoms in its backbone structure. Likewise, “lower alkenyl” and “lower alkynyl” have similar chain lengths.

“Heteroalkyl,” as used herein, refers to straight or branched chain, or cyclic carbon-containing alkyl radicals, or combinations thereof, containing at least one heteroatom. Suitable heteroatoms include, but are not limited to, O, N, Si, P and S, wherein the nitrogen, phosphorous and sulfur atoms are optionally oxidized, and the nitrogen heteroatom is optionally quaternized. For example, the term “heterocycloalkyl group” is a cycloalkyl group as defined above where at least one of the carbon atoms of the ring is substituted with a heteroatom such as, but not limited to, nitrogen, oxygen, sulphur, or phosphorus.

The term “alkenyl” as used herein is a hydrocarbon group of from 2 to 24 carbon atoms and structural formula containing at least one carbon-carbon double bond. Alkenyl groups include straight-chain alkenyl groups, branched-chain alkenyl, and cycloalkenyl. A cycloalkenyl is a non-aromatic carbon-based ring composed of at least three carbon atoms and at least one carbon-carbon double bond, such as a nonaromatic monocyclic or nonaromatic polycyclic ring containing 3-30 carbon atoms and at least one carbon-carbon double bond, 3-20 carbon atoms and at least one carbon-carbon double bond, or 3-10 carbon atoms and at least one carbon-carbon double bond in their ring structure, and have 5, 6 or 7 carbons and at least one carbon-carbon double bond in the ring structure. Cycloalkenyls containing a polycyclic ring system can have two or more non-aromatic rings in which two or more carbons are common to two adjoining rings (i.e., “fused cycloalkenyl rings”) and contain at least one carbon-carbon double bond. Asymmetric structures such as (AB)C═C(C′D) are intended to include both the E and Z isomers. This may be presumed in structural formulae herein wherein an asymmetric alkene is present, or it may be explicitly indicated by the bond symbol C. The term “alkenyl” as used throughout the specification, examples, and claims is intended to include both “unsubstituted alkenyls” and “substituted alkenyls,” the latter of which refers to alkenyl moieties having one or more substituents replacing a hydrogen on one or more carbons of the hydrocarbon backbone. The term “alkenyl” also includes “heteroalkenyl.”

The term “substituted alkenyl” refers to alkenyl moieties having one or more substituents replacing one or more hydrogen atoms on one or more carbons of the hydrocarbon backbone. Such substituents can be any substituents described above, e.g., halogen, azide, alkyl, aralkyl, alkenyl, alkynyl, cycloalkyl, hydroxyl, carbonyl (such as a carboxyl, alkoxycarbonyl, formyl, or an acyl), silyl, ether, ester, thiocarbonyl (such as a thioester, a thioacetate, or a thioformate), alkoxyl, phosphonium, phosphanyl, phosphoryl, phosphate, phosphonate, phosphinate, amino (e.g. quarternized amino), amido, amidine, imine, cyano, nitro, azido, oxo, sulfhydryl, alkylthio, sulfate, sulfonate, sulfamoyl, sulfonamido, sulfonyl, heterocyclyl, alkylaryl, haloalkyl, —CN, aryl, heteroaryl, polyaryl, polyheteroaryl, and combinations thereof.

“Heteroalkenyl,” as used herein, refers to straight or branched chain, or cyclic carbon-containing alkenyl radicals, or combinations thereof, containing at least one heteroatom. Suitable heteroatoms include, but are not limited to, O, N, Si, P and S, wherein the nitrogen, phosphorous and sulfur atoms are optionally oxidized, and the nitrogen heteroatom is optionally quaternized. For example, the term “heterocycloalkenyl group” is a cycloalkenyl group where at least one of the carbon atoms of the ring is substituted with a heteroatom such as, but not limited to, nitrogen, oxygen, sulphur, or phosphorus.

The term “alkynyl group” as used herein is a hydrocarbon group of 2 to 24 carbon atoms and a structural formula containing at least one carbon-carbon triple bond. Alkynyl groups include straight-chain alkynyl groups, branched-chain alkynyl, and cycloalkynyl. A cycloalkynyl is a non-aromatic carbon-based ring composed of at least three carbon atoms and at least one carbon-carbon triple bond, such as a nonaromatic monocyclic or nonaromatic polycyclic ring containing 3-30 carbon atoms and at least one carbon-carbon triple bond, 3-20 carbon atoms and at least one carbon-carbon triple bond, or 3-10 carbon atoms and at least one carbon-carbon triple bond in their ring structure, and have 5, 6 or 7 carbons and at least one carbon-carbon triple bond in the ring structure. Cycloalkynyls containing a polycyclic ring system can have two or more non-aromatic rings in which two or more carbons are common to two adjoining rings (i.e., “fused cycloalkynyl rings”) and contain at least one carbon-carbon triple bond. Asymmetric structures such as (AB)C≡C(C″D) are intended to include both the E and Z isomers. This may be presumed in structural formulae herein wherein an asymmetric alkyne is present, or it may be explicitly indicated by the bond symbol C. The term “alkynyl” as used throughout the specification, examples, and claims is intended to include both “unsubstituted alkynyls” and “substituted alkynyls,” the latter of which refers to alkynyl moieties having one or more substituents replacing a hydrogen on one or more carbons of the hydrocarbon backbone. The term “alkynyl” also includes “heteroalkynyl.”

The term “substituted alkynyl” refers to alkynyl moieties having one or more substituents replacing one or more hydrogen atoms on one or more carbons of the hydrocarbon backbone. Such substituents can be any substituents described above, e.g., halogen, azide, alkyl, aralkyl, alkenyl, alkynyl, cycloalkyl, hydroxyl, carbonyl (such as a carboxyl, alkoxycarbonyl, formyl, or an acyl), silyl, ether, ester, thiocarbonyl (such as a thioester, a thioacetate, or a thioformate), alkoxyl, phosphonium, phosphanyl, phosphoryl, phosphate, phosphonate, phosphinate, amino (e.g. quarternized amino), amido, amidine, imine, cyano, nitro, azido, sulfhydryl, alkylthio, sulfate, sulfonate, sulfamoyl, sulfonamido, sulfonyl, heterocyclyl, alkylaryl, haloalkyl, —CN, aryl, heteroaryl, polyaryl, polyheteroaryl, and combinations thereof.

The term “aryl” as used herein is any C₅-C₂₆ carbon-based aromatic group, heteroaromatic, fused aromatic, or fused heteroaromatic. For example, “aryl,” as used herein can include 5-, 6-, 7-, 8-, 9-, 10-, 14-, 18-, and 24-membered single-ring aromatic groups, including, but not limited to, benzene, naphthalene, anthracene, phenanthrene, chrysene, pyrene, corannulene, coronene, etc. “Aryl” further encompasses polycyclic ring systems having two or more cyclic rings in which two or more carbons are common to two adjoining rings (i.e., “fused aromatic rings”), wherein at least one of the rings is aromatic, e.g., the other cyclic ring or rings can be cycloalkyls, cycloalkenyls, cycloalkynyls, aryls and/or heterocycles. The aryl group can be substituted with one or more groups including, but not limited to, alkyl, alkynyl, alkenyl, aryl, halide, nitro, amino, ester, ketone, aldehyde, hydroxy, carboxylic acid, or alkoxy.

The term “substituted aryl” refers to an aryl group, wherein one or more hydrogen atoms on one or more aromatic rings are substituted with one or more substituents. Such substituents can be any substituents described above, e.g., halogen, azide, alkyl, aralkyl, alkenyl, alkynyl, cycloalkyl, hydroxyl, alkoxy, carbonyl (such as a ketone, aldehyde, carboxyl, alkoxycarbonyl, formyl, or an acyl), silyl, ether, ester, thiocarbonyl (such as a thioester, a thioacetate, or a thioformate), alkoxyl, phosphonium, phosphanyl, phosphoryl, phosphate, phosphonate, phosphinate, amino (e.g. quarternized amino), amido, amidine, imine, cyano, nitro, azido, sulfhydryl, imino, alkylthio, sulfate, sulfonate, sulfamoyl, sulfonamido, sulfonyl, heterocyclyl, alkylaryl, haloalkyl (such as CF3, —CH₂—CF₃, —CCl₃), —CN, aryl, heteroaryl, and combinations thereof.

The term “amino” as used herein includes the group

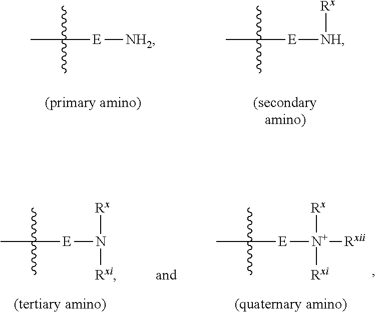

wherein, E is absent, or E is substituted or unsubstituted alkyl, substituted or unsubstituted alkenyl, substituted or unsubstituted alkynyl, substituted or unsubstituted aralkyl, substituted or unsubstituted aryl, wherein independently of E, R^{x}, R^{xi}, and R^{xii} each independently represent a substituted or unsubstituted alkyl, a substituted or unsubstituted alkenyl, a substituted or unsubstituted alkynyl, a substituted or unsubstituted carbonyl, a substituted or unsubstituted aryl, a substituted or unsubstituted heterocyclyl, a substituted or unsubstituted heteroaryl, a hydroxyl, a thiol, an amido, an amino, or —(CH₂)ₘ—R′″; R′″ represents a hydroxyl group, a substituted or unsubstituted carbonyl group, a substituted or unsubstituted aryl, a substituted or unsubstituted heterocyclyl, a substituted or unsubstituted aryl, a substituted or unsubstituted heteroaryl, an amido, or an amino; and m is zero or an integer ranging from 1 to 8. The term “quaternary amino” also includes the groups where the nitrogen, R^{x}, R^{xi}, and R^{xii} with the N⁺ to which they are attached complete a heterocyclyl or heteroaryl having from 3 to 14 atoms in the ring structure.

“Heterocycle” and “heterocyclyl” are used interchangeably, and refer to a cyclic radical attached via a ring carbon or nitrogen atom of a non-aromatic monocyclic or polycyclic ring containing 3-30 ring atoms, 3-20 ring atoms, 3-10 ring atoms, or 5-6 ring atoms, where each ring contains carbon and one to four heteroatoms each selected from the group consisting of non-peroxide oxygen, sulfur, and N(Y) wherein Y is absent or is H, O, C₁-C₁₀ alkyl, phenyl or benzyl, and optionally containing 1-3 double bonds and optionally substituted with one or more substituents. Heterocyclyl are distinguished from heteroaryl by definition. Heterocycles can be a heterocycloalkyl, a heterocycloalkenyl, a heterocycloalkynyl, etc, such as piperazinyl, piperidinyl, piperidonyl, 4-piperidonyl, dihydrofuro[2,3-b]tetrahydrofuran, morpholinyl, piperazinyl, piperidinyl, piperidonyl, 4-piperidonyl, piperonyl, pyranyl, 2H-pyrrolyl, 4H-quinolizinyl, quinuclidinyl, tetrahydrofuranyl, 6H-1,2,5-thiadiazinyl. Heterocyclic groups can optionally be substituted with one or more substituents as defined above for alkyl and aryl.

The term “heteroaryl” refers to C₅-C₃₀-membered aromatic, fused aromatic, biaromatic ring systems, or combinations thereof, in which one or more carbon atoms on one or more aromatic ring structures have been substituted with a heteroatom. Suitable heteroatoms include, but are not limited to, oxygen, sulfur, and nitrogen. Broadly defined, “heteroaryl,” as used herein, includes 5-, 6-, 7-, 8-, 9-, 10-, 14-, 18-, and 24-membered single-ring aromatic groups that may include from one to four heteroatoms, for example, pyrrole, furan, thiophene, imidazole, oxazole, thiazole, triazole, tetrazole, pyrazole, pyridine, pyrazine, pyridazine and pyrimidine, and the like. The heteroaryl group may also be referred to as “aryl heterocycles” or “heteroaromatics.” “Heteroaryl” further encompasses polycyclic ring systems having two or more rings in which two or more carbons are common to two adjoining rings (i.e., “fused rings”) wherein at least one of the rings is heteroaromatic, e.g., the other cyclic ring or rings can be cycloalkyls, cycloalkenyls, cycloalkynyls, aryls, heterocycles, or combinations thereof. Examples of heteroaryl rings include, but are not limited to, benzimidazolyl, benzofuranyl, benzothiofuranyl, benzothiophenyl, benzoxazolyl, benzoxazolinyl, benzthiazolyl, benztriazolyl, benztetrazolyl, benzisoxazolyl, benzisothiazolyl, benzimidazolinyl, carbazolyl, 4aH-carbazolyl, carbolinyl, chromanyl, chromenyl, cinnolinyl, decahydroquinolinyl, 2H,6H-1,5,2-dithiazinyl, furanyl, furazanyl, imidazolidinyl, imidazolinyl, imidazolyl, 1H-indazolyl, indolenyl, indolinyl, indolizinyl, indolyl, 3H-indolyl, isatinoyl, isobenzofuranyl, isochromanyl, isoindazolyl, isoindolinyl, isoindolyl, isoquinolinyl, isothiazolyl, isoxazolyl, methylenedioxyphenyl, naphthyridinyl, octahydroisoquinolinyl, 1,2,3-oxadiazolyl, 1,2,4-oxadiazolyl, 1,2,5-oxadiazolyl, 1,3,4-oxadiazolyl, oxazolidinyl, oxazolyl, oxindolyl, pyrimidinyl, phenanthridinyl, phenanthrolinyl, phenazinyl, phenothiazinyl, phenoxathinyl, phenoxazinyl, phthalazinyl, pteridinyl, purinyl, pyrazinyl, pyrazolidinyl, pyrazolinyl, pyrazolyl, pyridazinyl, pyridooxazole, pyridoimidazole, pyridothiazole, pyridinyl, pyridyl, pyrimidinyl, pyrrolidinyl, pyrrolinyl, pyrrolyl, quinazolinyl, quinolinyl, quinoxalinyl, tetrahydroisoquinolinyl, tetrahydroquinolinyl, tetrazolyl, 1,2,3-thiadiazolyl, 1,2,4-thiadiazolyl, 1,2,5-thiadiazolyl, 1,3,4-thiadiazolyl, thianthrenyl, thiazolyl, thienyl, thienothiazolyl, thienooxazolyl, thienoimidazolyl, thiophenyl and xanthenyl. One or more of the rings can be substituted as defined below for “substituted heteroaryl.”

The terms “amide” or “amido” are used interchangeably, refer to both “unsubstituted amido” and “substituted amido” and are represented by the general formula:

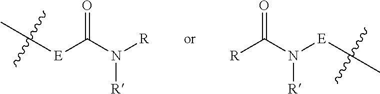

wherein, E is absent, or E is a substituted or unsubstituted alkyl, a substituted or unsubstituted alkenyl, a substituted or unsubstituted alkynyl, a substituted or unsubstituted aryl, substituted or unsubstituted heteroaryl, or a substituted or unsubstituted heterocyclyl, wherein independently of E, R and R′ each independently represent a hydrogen, a substituted or unsubstituted alkyl, a substituted or unsubstituted alkenyl, a substituted or unsubstituted alkynyl, a substituted or unsubstituted carbonyl, a substituted or unsubstituted heterocyclyl, a substituted or unsubstituted aryl, a substituted or unsubstituted heteroaryl, a hydroxyl, a thiol, an amido, an amino, or —(CH₂)ₘ—R′″, or R and R′ taken together with the N atom to which they are attached complete a heterocycle having from 3 to 14 atoms in the ring structure; R′″ represents a hydroxyl group, a substituted or unsubstituted carbonyl group, a substituted or unsubstituted aryl, a substituted or unsubstituted heterocyclyl, a substituted or unsubstituted aryl, a substituted or unsubstituted heteroaryl, an amido, or an amino; and m is zero or an integer ranging from 1 to 8. In some forms, when E is oxygen, a carbamate is formed.

“Carbonyl,” as used herein, is art-recognized and includes such moieties as can be represented by the general formula:

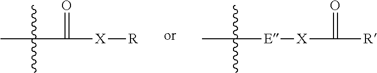

wherein X is a bond, or represents an oxygen or a sulfur, and R represents a hydrogen, a substituted or unsubstituted alkyl, a substituted or unsubstituted alkenyl, a substituted or unsubstituted alkynyl, a substituted or unsubstituted carbonyl, a substituted or unsubstituted heterocyclyl, a substituted or unsubstituted aryl, a substituted or unsubstituted heteroaryl, a hydroxyl, an amido, an amino, or —(CH₂)ₘ—R″, or a pharmaceutical acceptable salt; E″ is absent, or E″ is substituted or unsubstituted alkyl, substituted or unsubstituted alkenyl, substituted or unsubstituted alkynyl, substituted or unsubstituted aryl, substituted or unsubstituted heteroaryl, substituted or unsubstituted heterocyclyl; R′ represents a hydrogen, a substituted or unsubstituted alkyl, a substituted or unsubstituted alkenyl, a substituted or unsubstituted alkynyl, a substituted or unsubstituted carbonyl, a substituted or unsubstituted heterocyclyl, a substituted or unsubstituted aryl, a substituted or unsubstituted heteroaryl, a hydroxyl, an amido, an amino, or —(CH₂)ₘ—R″; R″ represents a hydroxyl group, a substituted or unsubstituted carbonyl group, a substituted or unsubstituted aryl, a substituted or unsubstituted heterocyclyl, a substituted or unsubstituted aryl, a substituted or unsubstituted heteroaryl, an amido, or an amino; and m is zero or an integer ranging from 1 to 8. Where X is oxygen and R′ is hydrogen, the formula represents a “formate.” Where X is oxygen and R or R′ is not hydrogen, the formula represents an “ester.” In general, where the oxygen atom of the above formula is replaced by a sulfur atom, the formula represents a “thiocarbonyl” group. Where X is sulfur and R or R′ is not hydrogen, the formula represents a “thioester.” Where X is sulfur and R is hydrogen, the formula represents a “thiocarboxylic acid.” Where X is sulfur and R′ is hydrogen, the formula represents a “thioformate.” Where X is a bond and R is not hydrogen, the above formula represents a “ketone.” Where X is a bond and R is hydrogen, the above formula represents an “aldehyde.”

The term “carboxyl” is defined by the formula —R^{iv}COOH, wherein R^{iv} is absent, a substituted or unsubstituted alkyl, a substituted or unsubstituted alkenyl, a substituted or unsubstituted alkynyl, a substituted or unsubstituted heterocyclyl, a substituted or unsubstituted aryl, or a substituted or unsubstituted heteroaryl.

The disclosed compounds and substituent groups, can, independently, possess two or more of the groups listed above. For example, if the compound or substituent group is a straight chain alkyl group, one of the hydrogen atoms of the alkyl group can be substituted with a hydroxyl group, an alkoxy group, etc. Depending upon the groups that are selected, a first group can be incorporated within second group or, alternatively, the first group can be pendant (i.e., attached) to the second group. For example, with the phrase “an alkyl group comprising an ester group,” the ester group can be incorporated within the backbone of the alkyl group. Alternatively, the ester can be attached to the backbone of the alkyl group. The nature of the group(s) that is (are) selected will determine if the first group is embedded or attached to the second group.

The compounds and substituents can be substituted with, independently, with the substituents described above in the definition of “substituted.”

Recitation of ranges of values herein are merely intended to serve as a shorthand method of referring individually to each separate value falling within the range, unless otherwise indicated herein, and each separate value is incorporated into the specification as if it were individually recited herein.

Use of the term “about” is intended to describe values either above or below the stated value in a range of approximately +/−10%; in other forms the values may range in value either above or below the stated value in a range of approximately +/−5%; in other forms the values may range in value either above or below the stated value in a range of approximately +/−2%; in other forms the values may range in value either above or below the stated value in a range of approximately +/−1%. The preceding ranges are intended to be made clear by context, and no further limitation is implied.

### II. Methods of Synthesis

To date, few synthetic routes for glycosylated amino acids, in particular optically pure glycosylated amino acids, such as glycosylated (2S,5R)-hydroxylysine, have been developed. Existing methods include i) racemic synthesis using HPLC resolution or a chiral resolving agent. This method requires HPLC purification, which is insufficient to provide large-scale desired product. Another approach is ii) asymmetric synthesis using a chiral auxiliary or chiral catalysis. Although these two strategies permit large-scale purification, the use of high toxicity reagents limits their application. Moreover, the final products of these two protocols are unsuitable for Fmoc solid phase peptide synthesis (Fmoc-SPPS), which precludes the possibility of glycopeptide synthesis. Another approach, iii) substrate directed asymmetric synthesis fails to convert an amino acid building block, such as a (2S,5R)-hydroxylysine, to a main chain Fmoc and side chain Boc protected product. The previously reported approaches remain unable to produce an Fmoc-SPPS applicable building block, which is important for the synthesis of glycosylated adiponectin collagenous domain.

Methods for the chemical synthesis of amino acid building blocks and glycosylated amino acids suitable for solid phase peptide synthesis, for example, Fmoc-SPPS, are disclosed. In particular, the methods can synthesize optically pure amino acid building blocks and glycosylated amino acids suitable for Fmoc-SPPS. For example, described herein are methods for the chemical synthesis of lysine building blocks and glycosylated lysine suitable for Fmoc-SPPS, such as (2S,5R)-hydroxylysine building block and glycosylated (2S,5R)-hydroxylysine.

The methods disclosed herein allow large scale synthesis of amino acid building blocks and glycosylated amino acids, in particular optically pure amino acid building blocks and glycosylated amino acids, such as (2S,5R)-hydroxylysine and glycosylated (2S,5R)-hydroxylysine, with Fmoc-SPPS compatible protecting groups. This method provides the following advantages: (1) in comparison with previously reported approaches, the method disclosed herein has fewer reaction steps and higher final product yield; (2) the orthogonal protecting groups in the building block make it a useful reagent for the glycopeptide synthesis, which was previously unobtainable; (3) the method avoids toxic and explosive reagents, such as diazo compound and sodium azide (NaN₃) used in previously reported approaches.

Methods for synthesizing peptides or proteins using the amino acid building blocks (e.g., (2S,5R)-hydroxylysine) and/or glycosylated amino acids (e.g., glycosylated 2S,5R)-hydroxylysine) are also provided. For example, methods for chemical synthesis of glycosylated adiponectin domains, such as the signal peptide, variable region, collagenous domain, and/or globular C1q head domain are described. In particular, methods for chemical synthesis of glycosylated adiponectin collagenous domains are disclosed. Also disclosed are methods for chemical synthesis of full-length glycosylated adiponectin, such as mammalian (e.g., human) adiponectin.

### A. Synthesis of Amino Acid Building Blocks

Methods for preparing amino acid building blocks are disclosed. In some forms, the amino acid building blocks prepared by the disclosed methods can have the structure of Formula (I):

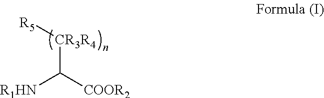

where n is an integer from 0 to 4; where each of R₁ and R₂ is independently a protecting group; where each occurrence of R₃ and R₄ is independently a hydrogen, a substituted alkyl, an unsubstituted alkyl, a hydroxyl, a thiol, or NR₁₃R₁₄, each of R₁₃ and R₁₄ is hydrogen, a substituted alkyl, or an unsubstituted alkyl; where R₅ is absent, a ketimine, an imidazole, an amino group, a carboxyl, a carboxylate, a hydroxyl, an amide, a thiol, a substituted alkyl, an unsubstituted alkyl, a sulfide, a substituted aryl, an unsubstituted aryl, or an indolizine, or R₅ is absent and (CR₃R₄)n is a substituted or unsubstituted alkyl that forms a ring with the nitrogen attached to C2 position, and when R₅ is or contains an amino group, a thiol group, and/or a hydroxyl group, the amino group, thiol group, and/or hydroxyl group is protected by a protecting group.

The protecting group refers to a chemical group or moiety that prevents reaction of a chemically reactive functional group under certain reaction conditions. The selection of protecting group depends on the type of chemically reactive group being protected and the specific reaction. In some forms, each occurrence of the protecting group of Formula (I) can be independently acetyl, benzoyl, benzyl (“Bn”), methyl benzyl, β-methoxyethoxymethyl ether, dimethoxytrityl, methoxymethyl ether, methoxytrityl[(4-methoxyphenyl)diphenylmethyl], p-methoxybenzyl ether, p-methoxyphenyl ether, methylthiomethyl ether, pivaloyl, tetrahydropyranyl, tetrahydrofuran, trityl, silyl ether, methyl ether, ethoxyethyl ether, carbobenzyloxy, p-methoxybenzyl carbonyl, tert-butyloxycarbonyl (“Boc”), 9-fluorenylmethyloxycarbonyl (“Fmoc”), carbamate, p-methoxybenzyl, 3,4-dimethoxybenzyl, p-methoxyphenyl, tosyl, trichloroethyl chloroformate, nosyl, nps, methyl ester, benzyl ester, tert-butyl ester, 2,6-disubstituted phenol ester, silyl ester, orthoester, or oxazoline.

For example, each occurrence of the protecting group of Formula (I) is independently acetyl, benzoyl, benzyl, methyl benzyl, tert-butyloxycarbonyl, 9-fluorenylmethyloxycarbonyl, carbamate, carbobenzyloxy, p-methoxybenzyl, 3,4-dimethoxybenzyl, or p-methoxyphenyl.

In some forms, the amino acid building blocks prepared by the disclosed methods can be optically pure, such as having the structure of Formula (XVI):

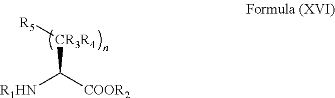

where n is an integer from 1 to 4; where each of R₁ and R₂ is independently a protecting group; where each occurrence of R₃ and R₄ is independently a hydrogen, a substituted alkyl, an unsubstituted alkyl, a hydroxyl, a thiol, or NR₁₃R₁₄, each of R₁₃ and R₁₄ is hydrogen, a substituted alkyl, or an unsubstituted alkyl; where R₅ is a ketimine, an imidazole, an amino group, a carboxyl, a carboxylate, a hydroxyl, an amide, a thiol, a substituted alkyl, an unsubstituted alkyl, a sulfide, a substituted aryl, an unsubstituted aryl, or an indolizine, and when R₅ is or contains an amino group, a thiol group, and/or a hydroxyl group, the amino group, thiol group, and/or hydroxyl group is protected by a protecting group; and where each occurrence of the protecting group is independently acetyl, benzoyl, benzyl, methyl benzyl, tert-butyloxycarbonyl, 9-fluorenylmethyloxycarbonyl, carbamate, carbobenzyloxy, p-methoxybenzyl, 3,4-dimethoxybenzyl, or p-methoxyphenyl.

In some forms, the amino acid building blocks prepared by the disclosed methods can have the structure of Formula (XVII):

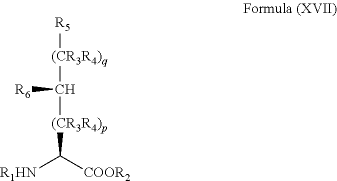

where each of p and q is an integer from 0 to 2 and p+q≤3; where each of R₁ and R₂ is independently a protecting group; where each occurrence of R₃ and R₄ is independently a hydrogen, a substituted alkyl, an unsubstituted alkyl, a hydroxyl, a thiol, or NR₁₃R₁₄, each of R₁₃ and R₁₄ is hydrogen, a substituted alkyl, or an unsubstituted alkyl; where R₅ is a ketimine, an imidazole, an amino group, a carboxyl, a carboxylate, a hydroxyl, an amide, a thiol, a substituted alkyl, an unsubstituted alkyl, a sulfide, a substituted aryl, an unsubstituted aryl, or an indolizine, and when R₅ is or contains an amino group, a thiol group, and/or a hydroxyl group, the amino group, thiol group, and/or hydroxyl group is protected by a protecting group; where R₆ is a substituted alkyl, an unsubstituted alkyl, a hydroxyl, a thiol, or NR₁₃R₁₄, each of R₁₃ and R₁₄ is hydrogen, substituted alkyl, or unsubstituted alkyl; and where each occurrence of the protecting group is independently acetyl, benzoyl, benzyl, methyl benzyl, tert-butyloxycarbonyl, 9-fluorenylmethyloxycarbonyl, carbamate, carbobenzyloxy, p-methoxybenzyl, 3,4-dimethoxybenzyl, or p-methoxyphenyl.

In some forms of Formulae (I), (XVI), and (XVII), R₅ is a ketimine, an imidazole, an amino group, a carboxyl, a carboxylate, a hydroxyl, an amide, a thiol, an indolizine, or a substituted aryl, wherein the substituent is a hydroxyl, a thiol, or an amino group.

In some forms, the amino acid building blocks prepared by the disclosed methods can have the structure of Formula (XVIII):

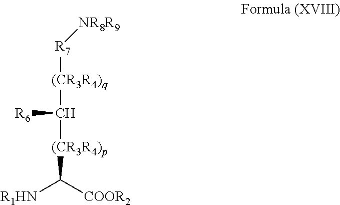

where each of p and q is an integer from 0 to 2 and p+q≤3; where each of R₁ and R₂ is independently a protecting group; where each occurrence of R₃ and R₄ is independently a hydrogen, a substituted alkyl, an unsubstituted alkyl, a hydroxyl, a thiol, or NR₁₃R₁₄, each of R₁₃ and R₁₄ is hydrogen, a substituted alkyl, or an unsubstituted alkyl; where R₆ is a substituted alkyl, an unsubstituted alkyl, a hydroxyl, a thiol, or NR₁₃R₁₄, each of R₁₃ and R₁₄ is hydrogen, substituted alkyl, or unsubstituted alkyl; where R₇ is absent, a hetero alkenyl that forms an imidazole with NR₈R₉, a carbonyl,

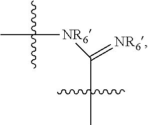

or an unsubstituted alkenyl that forms an indolizine with NR₈R₉, each occurrence of R₆′ is independently a hydrogen or a protecting group; where each of R₈ and R₉ is independently absent, a hydrogen, or a protecting group; and where each occurrence of the protecting group is independently acetyl, benzoyl, benzyl, methyl benzyl, tert-butyloxycarbonyl, 9-fluorenylmethyloxycarbonyl, carbamate, carbobenzyloxy, p-methoxybenzyl, 3,4-dimethoxybenzyl, or p-methoxyphenyl.

In some forms, the amino acid building blocks prepared by the disclosed methods can have the structure of Formula (XIX):

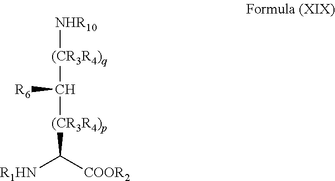

where each of p and q is an integer from 0 to 2 and p+q≤3; where each of R₁ and R₂ is independently a protecting group; where each occurrence of R₃ and R₄ is independently a hydrogen, a substituted alkyl, an unsubstituted alkyl, a hydroxyl, a thiol, or NR₁₃R₁₄, each of R₁₃ and R₁₄ is hydrogen, a substituted alkyl, or an unsubstituted alkyl; where R₆ is a substituted alkyl, an unsubstituted alkyl, a hydroxyl, a thiol, or NR₁₃R₁₄, each of R₁₃ and R₁₄ is hydrogen, substituted alkyl, or unsubstituted alkyl; where R₁₀ is a hydrogen or a protecting group; and where each occurrence of the protecting group is independently acetyl, benzoyl, benzyl, methyl benzyl, tert-butyloxycarbonyl, 9-fluorenylmethyloxycarbonyl, carbamate, carbobenzyloxy, p-methoxybenzyl, 3,4-dimethoxybenzyl, or p-methoxyphenyl.

In some forms, the amino acid building blocks prepared by the disclosed methods can have the structure of Formula (XX):

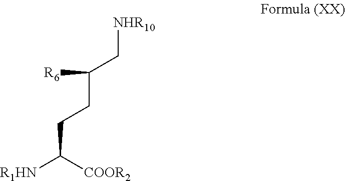

where each of R₁ and R₂ is independently a protecting group; where R₆ is a hydroxyl, a thiol, or NR₁₃R₁₄, each of R₁₃ and R₁₄ is hydrogen, a substituted alkyl, or an unsubstituted alkyl; where R₁₀ is a hydrogen or a protecting group; and where each occurrence of the protecting group is independently benzoyl, methyl benzyl, tert-butyloxycarbonyl, 9-fluorenylmethyloxycarbonyl, p-methoxybenzyl, or p-methoxyphenyl.

In some forms, the amino acid building block of Formula (I) and (XVI)-(XX) prepared by the disclosed methods can contains at least two different protecting groups, such as two different protecting groups or three different protecting groups described above. Typically, each of the protecting groups contained in the amino acid building blocks of Formula (I) and (XVI)-(XX) is compatible with solid phase peptide synthesis. For example, a protecting group is an SPPS compatible protecting group if it is stable under basic condition and instable under trifluoroacetic acid treatment.

For example, the amino acid building blocks prepared by the disclosed methods can have the structure of Formula (XXI), which contains three different protecting groups shown below:

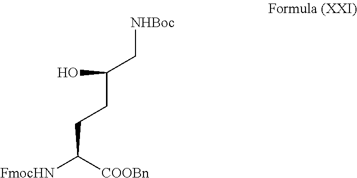

In some forms, methods for preparing an amino acid building block of any one of Formulae (I) and (XVI)-(XXI) can include: (i) performing a reaction between a first reactant of Formula (II) and a second reactant of Formula (III):

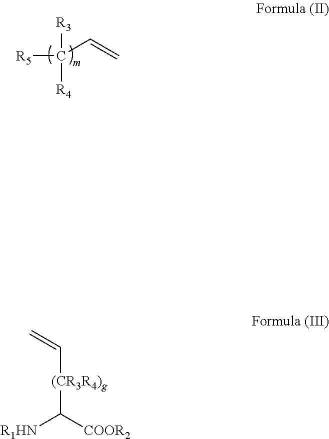

where each of m and g is an integer between 0 and 2 and R₁-R₅ are as defined above.

In some forms, the first reactant of Formula (II) can be prepared by converting a compound of Formula (IIa) to the first reactant of Formula (II):

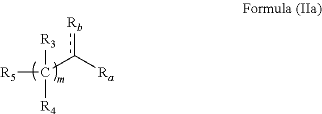

where m and R₃-R₅ are as defined above for Formula (II); Ra is hydrogen, hydroxyl, S—R_{c}, or substituted or unsubstituted alkenyl, such as a substituted or unsubstituted ethenyl, and R_{c} is a substituted or unsubstituted alkyl, such as methyl, ethyl, or propyl; and R_{b} is an oxygen, a hydroxyl, or a carbonyl. In some forms, the compound being converted to the first reactant of Formula (II) has the structure of Formula (IIb), (IIc), (IId), (IIe), or (IIf):

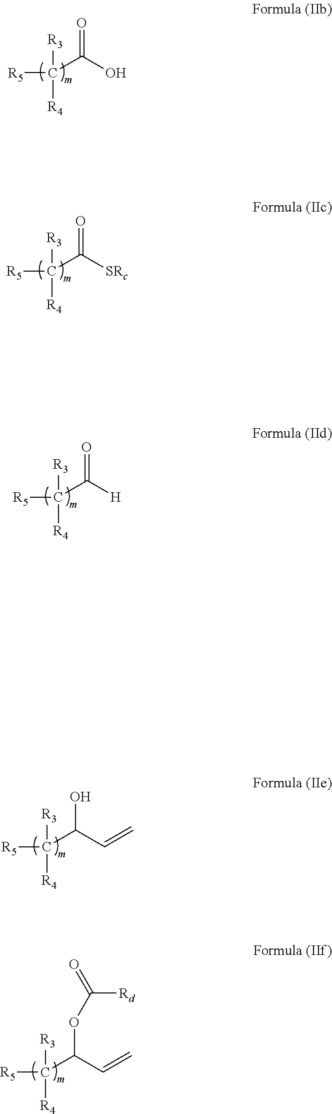

wherein m, R₃-R₅, and R_{c} are as defined above for Formula (II) and Formula (IIa); and R_{d} is a substituted or unsubstituted alkyl group, a substituted or unsubstituted aryl group, or an amino group. When R_{d} of Formula (IIf) is a substituted group, the substituents can be independently a substituted or unsubstituted alkyl, an amino group, a nitro, a hydroxyl, a thiol, or oxo. When R_{d} of Formula (IIf) is or contains an amino group, the amino group can be protected by a protecting group as described above, such as Boc. In some forms of Formula (IIf), the compound is a chiral compound having the structure of Formula (IIf′) or Formula (IIf″):

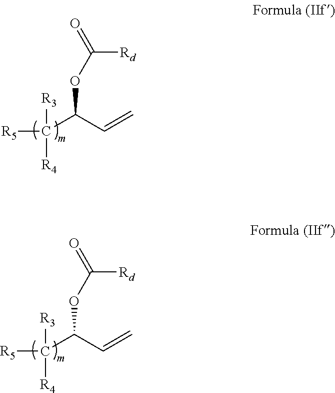

In some forms of Formula (IIf), the compound is a chiral compound having the structure of Formula (IIf′). In some forms of Formulae (IIf′) and (IIf″), R_{d} is

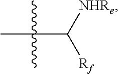

where Rₑ is a protecting group as described above, such as Boc, and R_{f} is a substituted or unsubstituted alkyl, such as methyl, ethyl, or propyl. In some forms of Formulae (IIf′) and (IIf″), R_{d} has at least one chiral center and can be

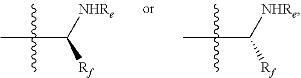

where Rₑ and R_{f} are as defined above.

In some forms of Formulae (IIa)-(IIf), (IIf′), and (IIf″), m is 1, R₃ and R₄ are hydrogen, and R₅ can be an amino group, where optionally the amino group is protected by a protecting group as described above, such as Boc.

In some forms of the reaction in step (i), the first reactant can have the structure of Formula (IV):

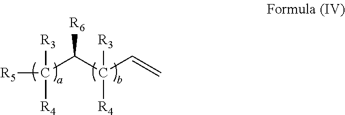

where each of a and b is independently 0 or 1; where each occurrence of R₃ and R₄ is independently a hydrogen, a substituted alkyl, an unsubstituted alkyl, a hydroxyl, a thiol, or NR₁₃R₁₄, each of R₁₃ and R₁₄ is hydrogen, a substituted alkyl, or an unsubstituted alkyl; where R₅ is a ketimine, an imidazole, an amino group, a carboxyl, a carboxylate, a hydroxyl, an amide, a thiol, a substituted alkyl, an unsubstituted alkyl, a sulfide, a substituted aryl, an unsubstituted aryl, or an indolizine, and when R₅ is or contains an amino group, a thiol group, and/or a hydroxyl group, the amino group, thiol group, and/or hydroxyl group is protected by any one of the protecting groups described above, such as acetyl, benzoyl, benzyl, methyl benzyl, tert-butyloxycarbonyl, 9-fluorenylmethyloxycarbonyl, carbamate, carbobenzyloxy, p-methoxybenzyl, 3,4-dimethoxybenzyl, and p-methoxyphenyl; and where R₆ is a substituted alkyl, an unsubstituted alkyl, a hydroxyl, a thiol, or NR₁₃R₁₄, each of R₁₃ and R₁₄ is hydrogen, substituted alkyl, or unsubstituted alkyl.

In some forms of Formula (IV), the sum of a and b is ≤2, such as 1 or 2.

In some forms of the reaction in step (i), the first reactant can have the structure of Formula (V):

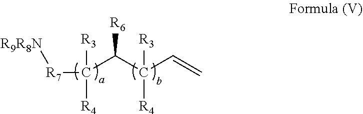

where each of a and b is independently 0 or 1; where each occurrence of R₃ and R₄ is independently a hydrogen, a substituted alkyl, an unsubstituted alkyl, a hydroxyl, a thiol, or NR₁₃R₁₄, each of R₁₃ and R₁₄ is hydrogen, a substituted alkyl, or an unsubstituted alkyl; where R₆ is a substituted alkyl, an unsubstituted alkyl, a hydroxyl, a thiol, or NR₁₃R₁₄, each of R₁₃ and R₁₄ is hydrogen, substituted alkyl, or unsubstituted alkyl; wherein R₇ is absent, a hetero alkenyl that forms an imidazole with NR₈R₉, a carbonyl,

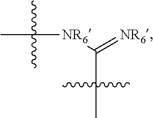

or an unsubstituted alkenyl that forms an indolizine with NR₈R₉, each occurrence of R₆′ is independently a hydrogen or a protecting group; where each of R₈ and R₉ is independently absent, a hydrogen, or a protecting group; and where each occurrence of the protecting group is independently acetyl, benzoyl, benzyl, methyl benzyl, tert-butyloxycarbonyl, 9-fluorenylmethyloxycarbonyl, carbamate, carbobenzyloxy, p-methoxybenzyl, 3,4-dimethoxybenzyl, or p-methoxyphenyl.

In some forms of the reaction in step (i), the first reactant can have the structure of Formula (VI):

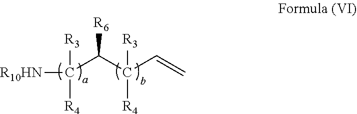

where each of a and b is independently 0 or 1; where each occurrence of R₃ and R₄ is independently a hydrogen, a substituted alkyl, an unsubstituted alkyl, a hydroxyl, a thiol, or NR₁₃R₁₄, each of R₁₃ and R₁₄ is hydrogen, a substituted alkyl, or an unsubstituted alkyl; where R₆ is a substituted alkyl, an unsubstituted alkyl, a hydroxyl, a thiol, or NR₁₃R₁₄, each of R₁₃ and R₁₄ is hydrogen, substituted alkyl, or unsubstituted alkyl; where R₁₀ is a hydrogen or a protecting group; and where each occurrence of the protecting group is independently acetyl, benzoyl, benzyl, methyl benzyl, tert-butyloxycarbonyl, 9-fluorenylmethyloxycarbonyl, carbamate, carbobenzyloxy, p-methoxybenzyl, 3,4-dimethoxybenzyl, or p-methoxyphenyl.

In some forms of the reaction in step (i), the first reactant can have the structure of Formula (VII):

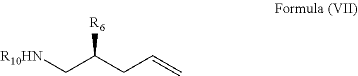

where R₆ is a hydroxyl, a thiol, or NR₁₃R₁₄, each of R₁₃ and R₁₄ is hydrogen, substituted alkyl, or unsubstituted alkyl; and where R₁₀ is a hydrogen or a protecting group, such as acetyl, benzoyl, benzyl, methyl benzyl, tert-butyloxycarbonyl, 9-fluorenylmethyloxycarbonyl, carbamate, carbobenzyloxy, p-methoxybenzyl, 3,4-dimethoxybenzyl, or p-methoxyphenyl.

In some forms of the reaction in step (i), the first reactant can have the structure of Formula (VIII):

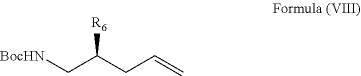

In some forms of the reaction in step (i), where the first reactant can have the structure of any one of Formulae (II) and (IV)-(VIII), the second reactant can have the structure of Formula (IX):

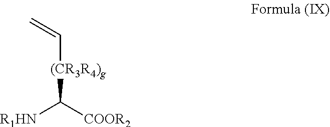

where g is an integer from 0 to 2; where each of R₁ and R₂ is independently a protecting group, such as acetyl, benzoyl, benzyl, methyl benzyl, tert-butyloxycarbonyl, 9-fluorenylmethyloxycarbonyl, carbamate, carbobenzyloxy, p-methoxybenzyl, 3,4-dimethoxybenzyl, or p-methoxyphenyl; and where each occurrence of R₃ and R₄ is independently a hydrogen, a substituted alkyl, an unsubstituted alkyl, a hydroxyl, a thiol, or NR₁₃R₁₄, each of R₁₃ and R₁₄ is hydrogen, a substituted alkyl, or an unsubstituted alkyl.

In some forms of the reaction in step (i), where the first reactant can have the structure of any one of Formulae (II) and (IV)-(VIII), the second reactant can have the structure of Formula (X):

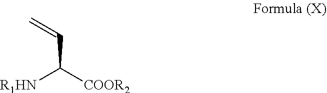

where each of R₁ and R₂ is independently a protecting group, such as acetyl, benzoyl, benzyl, methyl benzyl, tert-butyloxycarbonyl, 9-fluorenylmethyloxycarbonyl, carbamate, carbobenzyloxy, p-methoxybenzyl, 3,4-dimethoxybenzyl, and p-methoxyphenyl.

In some forms of the reaction in step (i), where the first reactant can have the structure of any one of Formulae (II) and (IV)-(VIII), the second reactant can have the structure of Formula (XI):

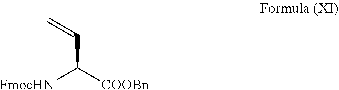

The method described above can produce amino acid building blocks of Formula (I) with a yield of at least 50%, at least 55%, at least 60%, at least 65%, at least 70%, or at least 75%. The yield of amino acid building blocks of Formula (I) produced using the disclosed method can be calculated by dividing the actual weight of product produced by the theoretical weight that could have been produced.

In some forms of the reaction performed in step (i), a catalyst is used. Typically, the catalyst used in the reaction performed in step (i) is non-toxic. Examples of catalyst that is suitable for use in the reaction performed in step (i) includes, but are not limited to, Grubbs II, CuW′ where W′ is a halogen, a Schrock catalyst, and a combination thereof.

The reaction performed in step (i) can be performed under a suitable condition, such as at a suitable temperature and pressure for a sufficient time period, to produce the amino acid building blocks of any one of Formula (I) and (XVI)-(XXI). Specific examples of performing the reaction in step (i) are described in the Examples.

In some forms, the methods for preparing the amino acid building block of any one of Formulae (I) and (XVI)-(XXI) does not include HPLC separation/purification.

In some forms, the methods for preparing the amino acid building block of any one of Formulae (I) and (XVI)-(XXI) can include a hydrogenation step subsequent to step (i). The hydrogenation step can reduce any unsaturated carbon-carbon bonds, such as carbon-carbon double bonds and/or triple bonds. Reaction conditions for performing hydrogenation reaction are known.

### B. Synthesis of Glycosylated Amino Acids

Methods for preparing glycosylated amino acids are disclosed. In some forms, the glycosylated amino acids prepared by the disclosed methods can have the structure of Formula (XII):

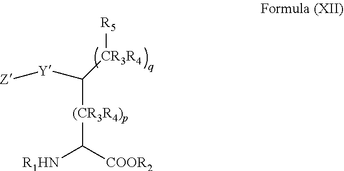

where each of p and q is an integer from 0 to 2; where Z′ is a monosaccharide moiety, a disaccharide moiety, an oligosaccharide moiety, or a polysaccharide moiety; where Y′ is an oxygen, a sulfur, or NR₁₅, R₁₅ is hydrogen, a substituted alkyl, or an unsubstituted alkyl; where each of R₁ and R₂ is independently a hydrogen or a protecting group; where each occurrence of R₃ and R₄ is independently a hydrogen, a substituted alkyl, an unsubstituted alkyl, a hydroxyl, a thiol, or NR₁₃R₁₄, each of R₁₃ and R₁₄ is hydrogen, a substituted alkyl, or an unsubstituted alkyl; where R₅ is absent, a ketimine, an imidazole, an amino group, a carboxyl, a carboxylate, a hydroxyl, an amide, a thiol, a substituted alkyl, an unsubstituted alkyl, a sulfide, a substituted aryl, an unsubstituted aryl, or an indolizine, and when R₅ is or contains an amino group, a thiol group, and/or a hydroxyl group, the amino group, thiol group, and/or hydroxyl group is protected by a protecting group; wherein each occurrence of the protecting group is independently acetyl, benzoyl, benzyl, methyl benzyl, β-methoxyethoxymethyl ether, dimethoxytrityl, methoxymethyl ether, methoxytrityl[(4-methoxyphenyl)diphenylmethyl], p-methoxybenzyl ether, p-methoxyphenyl ether, methylthiomethyl ether, pivaloyl, tetrahydropyranyl, tetrahydrofuran, trityl, silyl ether, methyl ether, ethoxyethyl ether, carbobenzyloxy, p-methoxybenzyl carbonyl, tert-butyloxycarbonyl, 9-fluorenylmethyloxycarbonyl, carbamate, p-methoxybenzyl, 3,4-dimethoxybenzyl, p-methoxyphenyl, tosyl, trichloroethyl chloroformate, nosyl, nps, methyl ester, benzyl ester, tert-butyl ester, 2,6-disubstituted phenol ester, silyl ester, orthoester, or oxazoline,

In some forms, the glycosylated amino acids prepared by the disclosed methods can have the structure of Formula (XXII) or Formula (XXIII):

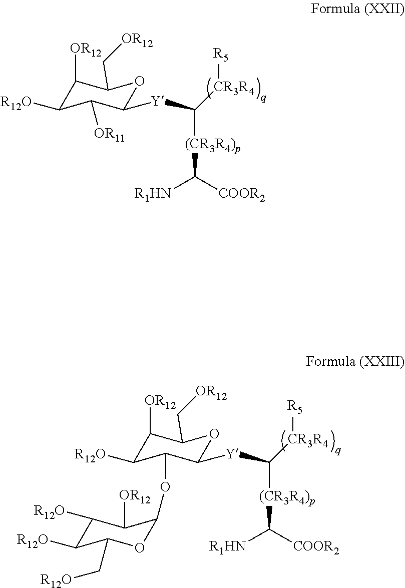

where each of R₁, R₂, R₁₁ and R₁₂ can be independently a hydrogen or a protecting group as described above, such as acetyl, benzoyl, benzyl, methyl benzyl, tert-butyloxycarbonyl, 9-fluorenylmethyloxycarbonyl, carbamate, carbobenzyloxy, p-methoxybenzyl, 3,4-dimethoxybenzyl, and p-methoxyphenyl, where R₃-R₅ and Y′ are as described above for Formula (XII).

In some forms, the glycosylated amino acids prepared by the disclosed methods can have the structure of Formula (XXIV) or Formula (XXV):

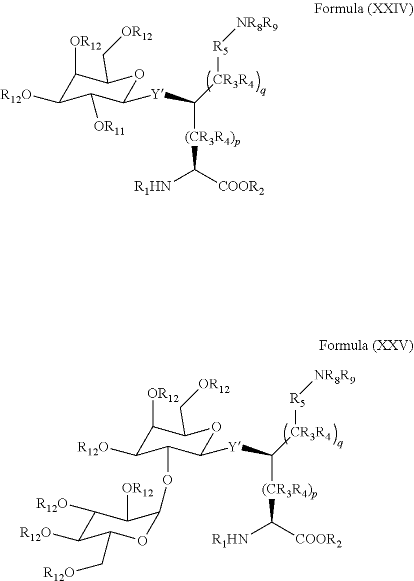

where each of R₁, R₂, R₁₁ and R₁₂ can be independently a hydrogen or a protecting group; where R₃-R₅ and Y′ are as described above for Formula (XII); where R₇ is absent, a hetero alkenyl that forms an imidazole with NR₈R₉, a carbonyl,

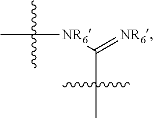

or an unsubstituted alkenyl that forms an indolizine with NR₈R₉, each occurrence of R₆′ is independently a hydrogen or a protecting group; where each of R₈ and R₉ is independently absent, a hydrogen, or a protecting group; and where each occurrence of the protecting group is independently acetyl, benzoyl, benzyl, methyl benzyl, tert-butyloxycarbonyl, 9-fluorenylmethyloxycarbonyl, carbamate, carbobenzyloxy, p-methoxybenzyl, 3,4-dimethoxybenzyl, or p-methoxyphenyl.

In some forms, the glycosylated amino acids prepared by the disclosed methods can have the structure of Formula (XXVI) or Formula (XXVII):

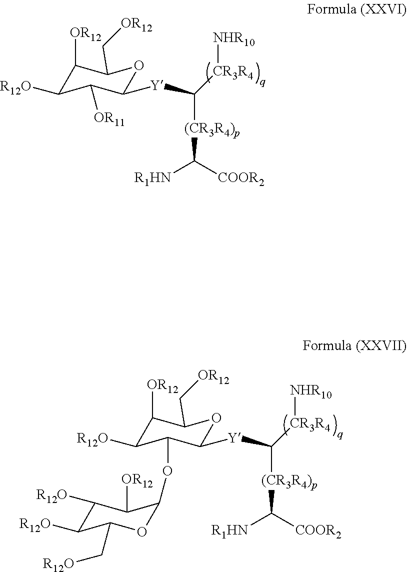

where each of R₁, R₂, R₁₁ and R₁₂ can be independently a hydrogen or a protecting group; where R₃-R₅ and Y′ are as described above for Formula (XII); where R₁₀ can be a hydrogen or a protecting group; and where each occurrence of the protecting group is independently acetyl, benzoyl, benzyl, methyl benzyl, tert-butyloxycarbonyl, 9-fluorenylmethyloxycarbonyl, carbamate, carbobenzyloxy, p-methoxybenzyl, 3,4-dimethoxybenzyl, or p-methoxyphenyl.

In some forms, the glycosylated amino acids prepared by the disclosed methods can have the structure of Formula (XXVIII) or Formula (XXIX):

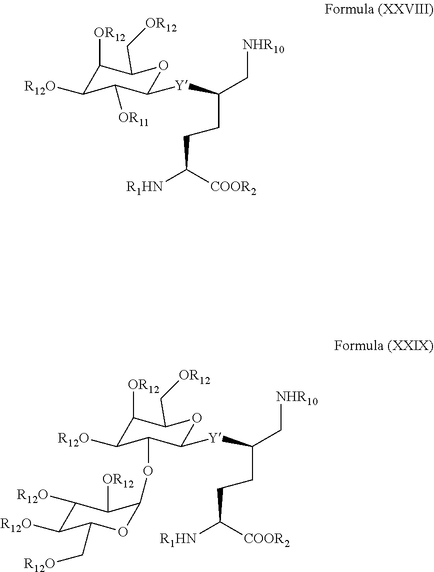

where each of R₁, R₂, R₁₁ and R₁₂ can be independently a hydrogen or a protecting group; where Y′ is as described above for Formula (XII); where R₁₀ can be a hydrogen or a protecting group; and where each occurrence of the protecting group is independently acetyl, benzoyl, benzyl, methyl benzyl, tert-butyloxycarbonyl, 9-fluorenylmethyloxycarbonyl, carbamate, carbobenzyloxy, p-methoxybenzyl, 3,4-dimethoxybenzyl, or p-methoxyphenyl.

In some forms, the glycosylated amino acids of Formula (VII) and (XII)-(XXIX) prepared by the disclosed methods can contains at least two different protecting groups, such as two different protecting groups or three different protecting groups described above. Typically, each of the protecting groups contained in the glycosylated amino acids of Formula (VII) and (XII)-(XXIX) is compatible with solid phase peptide synthesis.

For example, the glycosylated amino acids prepared by the disclosed methods can have the structure of Formula (XXX) or Formula (XXXI):

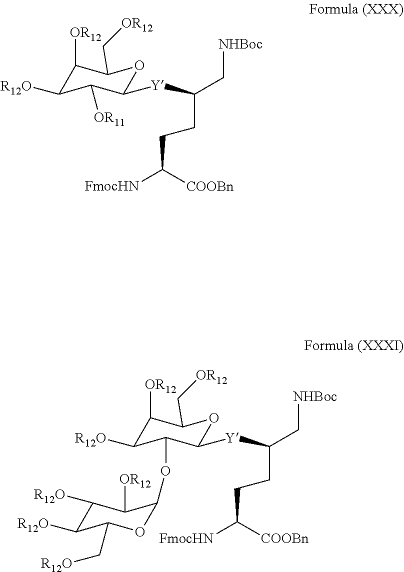

where each of R₁₁ and R₁₂ can be independently a hydrogen or a protecting group; where Y′ is as described above for Formula (XII); and where each occurrence of the protecting group is independently acetyl, benzoyl, benzyl, methyl benzyl, tert-butyloxycarbonyl, 9-fluorenylmethyloxycarbonyl, carbamate, carbobenzyloxy, p-methoxybenzyl, 3,4-dimethoxybenzyl, or p-methoxyphenyl.

In some forms, the glycosylated amino acids prepared by the disclosed methods can have the structure of Formula (XXXI) as described above.

In some forms of the glycosylated amino acids of Formula (XII) and (XXII)-(XXXI), Y′ is O or S, and R₁₁ is different R₁₂. In some forms of the glycosylated amino acids of Formula (XII) and (XXII)-(XXXI), Y′ is O or S, R₁ is acetyl or p-methoxybenzyl and R₁₂ is methyl benzyl, such as 4-methyl benzyl.

In some forms, methods for preparing an amino acid building block of any one of Formulae (XII) and (XXII)-(XXXI) can include: (ii) performing a reaction between a saccharide of Formula (XIII) and an amino acid building block of Formula (I):

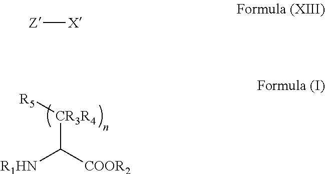

where Z′ and R₁-R₅ are as defined above; where n is an integer from 0 to 4; and wherein X′ is a leaving group.

In some forms of the reaction in step (ii), the saccharide can have the structure of Formula (XIV) or Formula (XV):

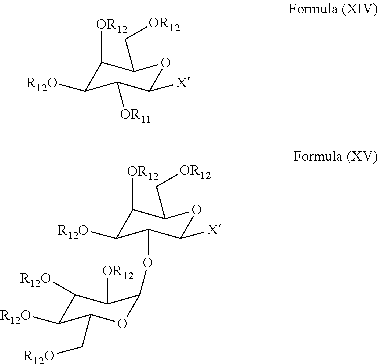

where each occurrence of R₁ and R₁₂ is independently a hydrogen or a protecting group, such as acetyl, benzoyl, benzyl, methyl benzyl, tert-butyloxycarbonyl, 9-fluorenylmethyloxycarbonyl, carbamate, carbobenzyloxy, p-methoxybenzyl, 3,4-dimethoxybenzyl, or p-methoxyphenyl.

In some forms of the reaction in step (ii), the leaving group X′ of the saccharide can be a dinitrogen, a dialkyl ether, a perfluoroalkylsulfonate, tosylate, mesylate, a halogen, SR₁₆, OR₁₇, a thioether, an amino group, a carboxylate, a phenoxide, or an amide, and where each of R₁₆ and R₁₇ is independently a hydrogen, a substituted alkyl, an unsubstituted alkyl, a substituted aryl, an unsubstituted aryl, an imino, or a carbonyl.

In some forms of the reaction in step (ii), the leaving group X′ of the saccharide can be a halogen, SR₁₆, OR₁₇, or NR₁₈R₁₉, and where each of R₁₆-R₁₉ is independently a hydrogen, a substituted alkyl, an unsubstituted alkyl, a substituted aryl, an unsubstituted aryl, an imino, or a carbonyl.

In some forms of the reaction in step (ii), where the saccharide can have the structure of any one of Formulae (VIII), (XIV) and (XV), the amino acid building block can have the structure of any one of Formula (XVI)-(XXI) as described above.

The method described above for preparing glycosylated amino acids can be performed in a suitable solvent. Examples of solvents suitable for performing the reaction in step (ii) include, but are not limited to, ethyl ether, tetrahydrofuran, acetonitrile, propionitrile, and toluene. For example, the reaction performed in step (ii) can be performed in a mixture of dimethylformamide and dichloromethane.

In some forms of the reaction performed in step (ii), a catalyst is used. Typically, the catalyst used in the reaction performed in step (ii) is non-toxic. Examples of catalyst that is suitable for use in the reaction performed in step (ii) includes, but are not limited to, toluenesulfenyl chloride (TolSCl), silver trifluoromethanesulfonate (AgOTf), N-Iodosuccinimide, trimethylsilicon trifluoromethesulfonate, Au catalyst, trifluoromethanesulfonic acid, and a combination thereof. For example, the reaction in step (ii) is performed using a catalyst that is a mixture of TolSCl and AgOTf.

The reaction performed in step (ii) can be performed under a suitable condition, such as at a suitable temperature and pressure for a sufficient time period, to produce the glycosylated amino acids of any one of Formula (XII) and (XXII)-(XXXI). Specific examples of performing the reaction in step (ii) are described in the Examples.

In some forms, the methods for preparing the amino acid building block of any one of Formulae (XII) and (XXII)-(XXXI) does not include HPLC separation/purification.

The method described above can produce glycosylated amino acids of any one of Formula (XII) and (XXII)-(XXXI) with a yield of at least 30%, at least 35%, at least 40%, at least 45%, or at least 50%. The yield of glycosylated amino acids produced using the disclosed method can be calculated by dividing the actual weight of product produced by the theoretical weight that could have been produced.

In some forms, the methods for preparing the glycosylated amino acids of any one of Formula (XII) and (XXII)-(XXXI) can include a deprotection step subsequent to step (ii). The deprotection step can selectively remove one or more of the protecting groups on the produced glycosylated amino acids of any one of Formula (XII) and (XXII)-(XXXI). For example, the deprotection step selectively removes the protecting group on the carboxyl group and thus the carboxylic acid of the glycosylated amino acid retains reactivity for any subsequent reactions, such as peptide synthesis. For example, when R₂ of Formula (VII) and (XII)-(XXIX) is a protecting group, the deprotection step removes R₂ and thus produces the carboxylic acid group on the glycosylated amino acid. Reaction conditions for performing deprotection of selected groups are known.

In some forms, the amino acid building block having the structure of any one of Formulae (I) and (XVI)-(XXI) in step (ii) is prepared by the method described in the synthesis of amino acid building block, i.e. by the reaction in step (i).

Exemplary substituents suitable for any of the substituted groups described above can be a substituted or unsubstituted alkyl, a substituted or unsubstituted alkenyl, a substituted or unsubstituted alkynyl, a substituted or unsubstituted heterocyclyl, a substituted or unsubstituted aryl, a substituted or unsubstituted heteroaryl, a substituted or unsubstituted polyaryl, a substituted or unsubstituted polyheteroaryl, a substituted or unsubstituted aralkyl, a substituted or unsubstituted carbonyl, a substituted or unsubstituted alkoxy, a halogen, a hydroxyl, a phenoxy, an aroxy, an alkylthio, a phenylthio, an arylthio, a cyano, an isocyano, a nitro, an carboxyl, an amino, an amido, an oxo, a silyl, a sulfinyl, a sulfonyl, a sulfonic acid, a phosphonium, a phosphanyl, a phosphoryl, a phosphonyl, a thiol, an amino acid, a peptide, a polypeptide, or a sugar group (such as a glucose group or an acetylated glucose), or a combination thereof.

The alkyl described above can be a linear alkyl, a branched alkyl, or a cyclic alkyl (either monocyclic or polycyclic). Exemplary alkyl include a linear C₁-C₃₀ alkyl, a branched C₄-C₃₀ alkyl, a cyclic C₃-C₃₀ alkyl, a linear C₁-C₂₀ alkyl, a branched C₄-C₂₀ alkyl, a cyclic C₃-C₂₀ alkyl, a linear C₁-C₁₀ alkyl, a branched C₄-C₁₀ alkyl, a cyclic C₃-C₁₀ alkyl, a linear C₁-C₆ alkyl, a branched C₄-C₆ alkyl, a cyclic C₃-C₆ alkyl, a linear C₁-C₄ alkyl, cyclic C₃-C₄ alkyl, such as a linear C₁-C₁₀, C₁-C₉, C₁-C₈, C₁-C₇, C₁-C₆, C₁-C₅, C₁-C₄, C₁-C₃, C₁-C₂ alkyl group, a branched C₃-C₉, C₃-C₉, C₃-C₅, C₃-C₇, C₃-C₆, C₃-C₅, C₃-C₄ alkyl group, or a cyclic C₃-C₉, C₃-C₉, C₃-C₈, C₃-C₇, C₃-C₆, C₃-C₅, C₃-C₄ alkyl group.

It is understood that any of the above-described exemplary alkyl groups can be heteroalkyl. For example, the alkyl can be a linear C₂-C₃₀ heteroalkyl, a branched C₄-C₃₀ heteroalkyl, a cyclic C₃-C₃₀ heteroalkyl (i.e. a heterocycloalkyl), a linear C₁-C₂₀ heteroalkyl, a branched C₄-C₂₀ heteroalkyl, a cyclic C₃-C₂₀ heteroalkyl, a linear C₁-C₁₀ heteroalkyl, a branched C₄-C₁₀ heteroalkyl, a cyclic C₃-C₁₀ heteroalkyl, a linear C₁-C₆ heteroalkyl, a branched C₄-C₆ heteroalkyl, a cyclic C₃-C₆ heteroalkyl, a linear C₁-C₄ heteroalkyl, cyclic C₃-C₄ heteroalkyl, such as a linear C₁-C₁₀, C₁-C₉, C₁-C₅, C₁-C₇, C₁-C₆, C₁-C₅, C₁-C₄, C₁-C₃, C₁-C₂ heteroalkyl group, a branched C₃-C₉, C₃-C₉, C₃-C₅, C₃-C₇, C₃-C₆, C₃-C₅, C₃-C₄ heteroalkyl group, or a cyclic C₃-C₉, C₃-C₉, C₃-C₅, C₃-C₇, C₃-C₆, C₃-C₅, C₃-C₄ heteroalkyl group.

The aryl group described above can be a C₅-C₃₀ aryl, a C₅-C₂₀ aryl, a C₅-C₁₂ aryl, a C₅-C₁₁ aryl, a C₅-C₉ aryl, a C₆-C₂₀ aryl, a C₆-C₁₂ aryl, a C₆-C₁₁ aryl, or a C₆-C₉ aryl. It is understood that the aryl can be a heteroaryl, such as a C₅-C₃₀ heteroaryl, a C₅-C₂₀ heteroaryl, a C₅-C₁₂ heteroaryl, a C₅-C₁₁ heteroaryl, a C₅-C₉ heteroaryl, a C₆-C₃₀ heteroaryl, a C₆-C₂₀ heteroaryl, a C₆-C₁₂ heteroaryl, a C₆-C₁₁ heteroaryl, or a C₆-C₉ heteroaryl.

### C. Synthesis of Glycosylated Peptide Via SPPS and Ligation

Methods for the chemical synthesis of glycosylated peptides containing one or more glycosylated amino acid building blocks are provided. For example, disclosed are methods for the chemical synthesis of glycosylated peptides containing one or more glycosylated (2S,5R)-hydroxylysines. In particular, disclosed are methods for the chemical synthesis of adiponectin-based glycopeptides containing one or more glycosylated (2S,5R)-hydroxylysines, such as a glycopeptide including the sequence of the human adiponectin collagenous domain, wherein one or more of the lysine residues in the collagenous domain are glycosylated (2S,5R)-hydroxylysines.

The peptides can be produced by stepwise synthesis or by synthesis of a series of fragments that can be coupled by well-known techniques. In some preferred forms, the synthesis of a final glycosylated peptide is performed via ligating two pre-cursor peptide fragments to form the final peptide. The glycosylated amino acid building block(s) is incorporated into the precursor peptide fragment(s) at the desired position. The precursor peptide fragment(s) containing the glycosylated amino acid building block (e.g., glycosylated (2S,5R)-hydroxylysine) can be synthesized via solid phase peptide synthesis.

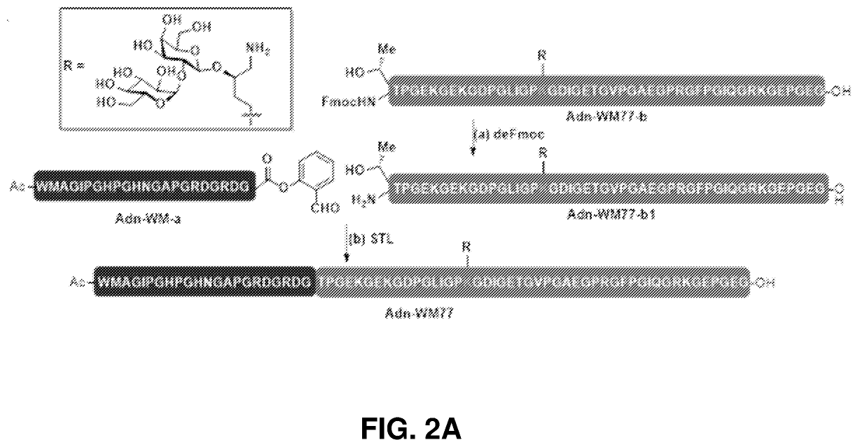

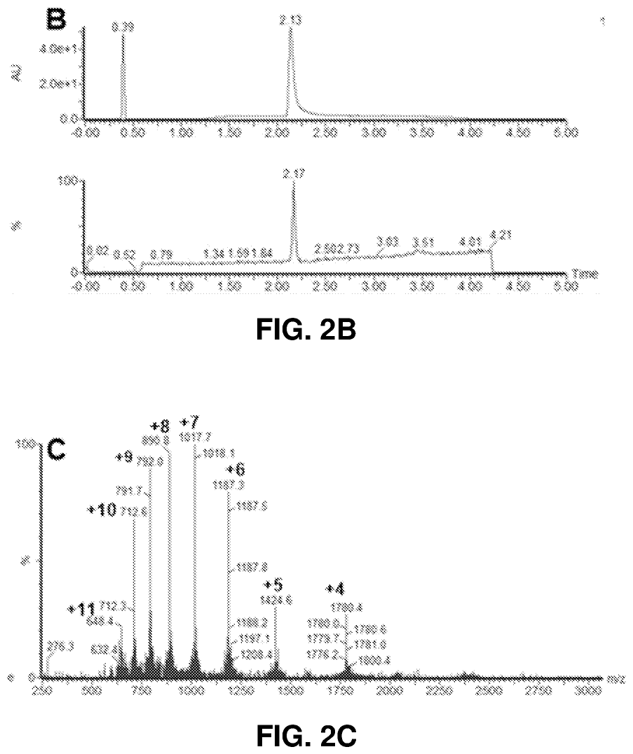

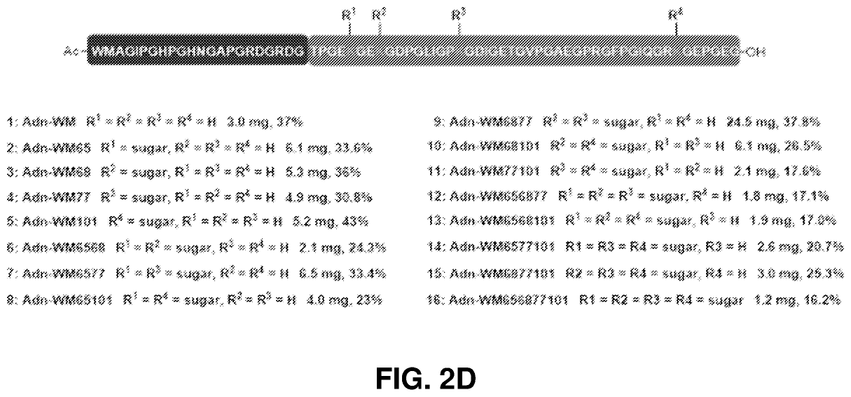

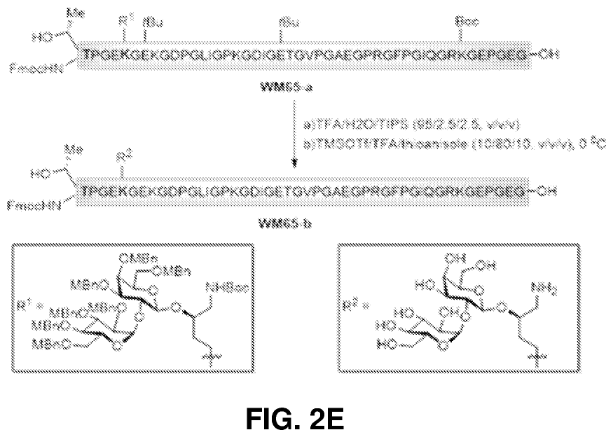

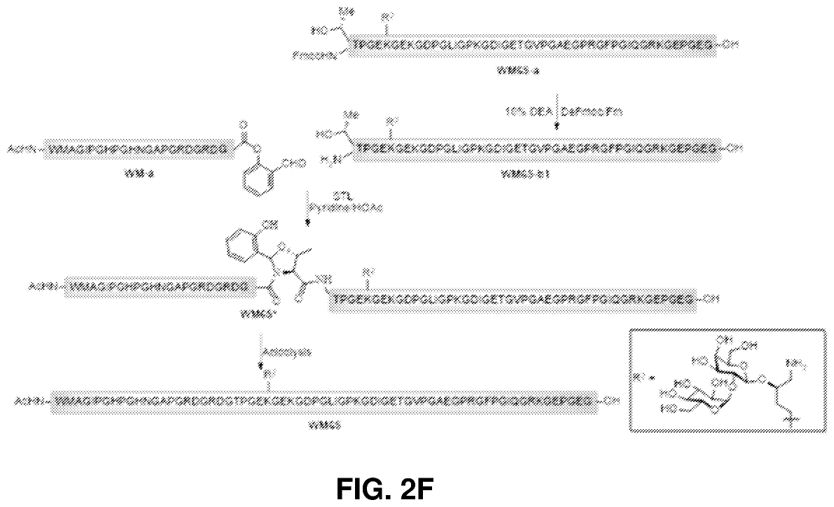

In preferred forms, the peptide fragments are ligated via Ser/Thr ligation. The Ser/Thr ligation approach is known in the art. See, for example, References 32-35 and FIGS. 2A-2C. This method allows for ligation at N-terminal serine and threonine residues to generate Xaa-Ser/Thr linkages directly, using serine or threonine residues at the reacting N-terminus.

In certain forms, the peptide fragments to be ligated are chemically synthesized by any of a number of fluid or solid phase peptide synthesis techniques known to those of skill in the art. For example, standard Fmoc synthesis is described in the literature (e.g., solid phase peptide synthesis, see E. Atherton, RC Sheppard, Oxford University press (1989), or liquid phase synthesis (where peptides are assembled using a mixed strategy by BOC chemistry and fragment condensation).

Preferably, the peptide fragments to be ligated can be prepared via solid phase peptide synthesis (SPPS). Methods for SPPS are well known in the art. Solid phase synthesis, in which the C-terminal amino acid of the sequence is attached to an insoluble support followed by sequential addition of the remaining amino acids in the sequence is a preferred method for the chemical synthesis of the peptides. Techniques for solid phase synthesis are well known to those of skill in the art and are described, for example, by Barany and Merrifield (1963) Solid-Phase Peptide Synthesis; pp. 3-284 in The Peptides: Analysis, Synthesis, Biology. Vol. 2: Special Methods in Peptide Synthesis, Part A.; Merrifield et al. (1963) J. Am. Chem. Soc, 85: 2149-2156, and Stewart et al. (1984) Solid Phase Peptide Synthesis, 2nd ed. Pierce Chem. Co., Rockford, 111.

Such methods include bench scale solid phase synthesis and automated peptide synthesis in any one of the many commercially available peptide synthesizers. Solid phase synthesis is commonly used, and various commercial synthesizers are available, such as automated synthesizers by Applied Biosystems Inc., Foster City, CA; Beckman; MultiSyntech, Bochum, Germany etc. Functional groups for conjugating the peptide to small molecules, label moieties, peptides, or proteins may be introduced into the molecule during chemical synthesis. In addition, small molecules and label moieties/reporter units may be attached during the synthetic process. Preferably, introduction of the functional groups and conjugation to other molecules minimally affects the structure and function of the subject peptide.

Chemical synthesis typically starts from the C-terminus, to which amino acids are sequentially added using, for example, a 2-chlorotrityl chloride resin, a Rink amide resin (resulting in an —NH₂ group at the C-terminus of the peptide after cleavage from the resin), or a Wang resin (resulting in an —OH group at the C-terminus). Accordingly, peptides having a C-terminal moiety that may be selected from the group consisting of —H, —OH, —COOH, —CONH₂, and —NH₂ are contemplated for use.

Standard Fmoc (9-florenylmethoxycarbonyl) derivatives include Fmoc-Asp(OtBu)-OH, Fmoc-Arg(Pbf)-OH, and Fmoc-Ala-OH. Couplings are mediated with DIC (diisopropylcarbodiimide)/6-Cl-HOBT (6-chloro-1-hydroxybenzotriazole). In some forms, the last four residues of the peptide require one or more recoupling procedures. In particular, the final Fmoc-Arg(Pbf)-OH coupling may require recoupling. For example, a second or third recoupling can be carried out to complete the peptide using stronger activation chemistry such as DIC/HOAT (1-hydroxy-7-azabenzotriazole) or HATU (1-[bis(dimethylamino)methylene]-1H-1,2,3-triazolo[4,5-b]pyridinium 3-oxid hexafluorophosphate)/NMM (N-methylmorpholine).

Acidolytic cleavage of the peptide can be carried out with the use of carbocation scavengers (thioanisole, anisole and H₂O). Optimization can be achieved by varying the ratio of the components of the cleavage mixture. An exemplary cleavage mixture ratio is 90:2.5:2.5:5 (TFA-thioanisole-anisole-H₂O). The reaction can be carried out for 4 hours at room temperature. In some forms, the removal of residual impurities is carried out by wash steps. For example, trifluoroacetic acid (TFA) and organic impurities can be eliminated by precipitation and repeated washes with cold diethyl ether and methyl t-butyl ether (MTBE).

As shown in the working examples, peptide salicylaldehyde esters (SAL), which are compatible with standard SPPS strategies are prepared. Thus, in some forms, fully protected peptides bearing C-terminal free carboxylic acid and N-terminal Ac are synthesized via Fmoc-SPPS using 2-chlorotrityl chloride resin. The fully protected peptidic acid can be coupled with 2-(dimethoxymethyl)-phenol 12 in anhydrous CH₂Cl₂ overnight, in the presence of N,N′-dicyclohexylcarbodiimide (DCC) and 4-dimethylaminopyridine (DMAP) (3.3 mg, 2.7 μmol). Upon completion, the reaction mixture can be concentrated and subjected to the treatment with TFA/H₂O (95/5, v/v). The crude peptide SAL ester can be precipitated out by cold diethyl ether. After decanting diethyl ether, the remaining solid can be dissolved in 15% CH₃CN/H₂O and subjected to preparative HPLC purification (e.g., 10-50% CH₃CN/H₂O over 30 min).

Synthesis of Peptide hAdn-WM65

By way of example, the chemical synthesis of a specific adiponectin glycoform, hAdn-WM65 (see Table 3), is described below. It is understood that this method is generally applicable to the chemical synthesis of any glycopeptide disclosed herein or known in the art.

### Preparation of WM65-b

Peptide WM65-a was synthesized using SPPS with a 2-chlorotrityl chloride resin by introducing the glycosylated 5-hydroxylysine building-block at the desired position (lysine residue 65 of human adiponectin; in the collagenous domain). Upon completion of synthesis, the crude peptide was then deprotected and cleaved from the resin using 5 mL TFA/TIPS/H₂O (90:5:5, v/v/v) for 1 h. The resin was filtered and the combined filtrate was stirred at −10° C. in cooling bath for 10 min. Then, thioanisole 625 μL was added to the above solution and stirred for an additional 10 min. TMSOTf 625 μL was slowly added to the above solution and stirred at −10° C. for 45 min. The reaction solution was poured into cold (−20° C.) diethyl ether (45 mL), and the resulting suspension was centrifuged to give a white pellet. After decanting diethyl ether, the remaining solid was dissolved in 2 mL 50% CH₃CN/H₂O and NH₄HCO₃ solid was added until the solution's pH became 7. The resulting solution was stirred at room temperature for 1 h and subjected to preparative HPLC purification (20-50% CH₃CN/H₂O over 45 min). Lyophilization afforded WM65-b (13.0 mg, 10.2%) as a white powder. See FIG. 2E.

### Preparation of WM65

WM65-b (13.0 mg, 2.55 μmol) was dissolved in 250 μL CH₃CN/H₂O/diethylamine (4.5/4.5/1, v/v/) at room temperature and stirred at room temperature for 2 h to give product WM65-b1. See FIG. 2F. The reaction mixture was diluted with 50% CH₃CN/H₂O (20 mL) and subjected to lyophilization to produce WM65-b1 as a slightly yellow solid. This crude peptide was washed by diethyl ether. The crude WM65-b1 and WM-a (6.0 mg, 2.55 μmol) were dissolved in pyridine/acetic acid (1/1, mole/mole) buffer at a concentration of 15 mM at room temperature. The reaction mixture was stirred at room temperature for 4 h to give ligation intermediate WM65*, and the solvent was then blown off under a stream of condensed N₂. The residue was treated with 1 mL TFA/H₂O (95/5, v/v) for 20 min to obtain crude product WM65, and then TFA was blown off under a stream of condensed N₂. The remaining residue was subjected to preparative HPLC purification (10-35% CH₃CN/H₂O over 45 min) and lyophilization to provide hAdn-WM65 (6.1 mg, 34%) as a white powder. See FIG. 2F.

### Peptide Purification

In some forms, the final peptide product is purified. Peptides produced using the disclosed methods can be purified using high pressure liquid chromatography (HPLC). Suitable solvents for dissolving the peptides include neat TFA. Typically, the peptides remain soluble at TFA concentrations of 0.5% to 8% and can be loaded onto reverse phase (RP)-HPLC columns for salt exchange. Exemplary salt exchange methods use 3-4 column volumes of acidic buffer to wash away the TFA counter ion due to its stronger acidity coefficient. Buffers suitable for use in washing away the TFA counter ion include 0.1% HCl in H₂O.

Following removal of TFA, peptides can be eluted with a step gradient. Exemplary elution buffers include 30% acetonitrile (MeCN) vs. 0.1% HCl in H₂O. For acetate exchange, peptides can be loaded from the same diluted TFA solution, washed with 3-4 column volumes of 1% acetic acid (AcOH) in H₂O, followed by 2 column volumes of 0.1 M NH₄OAc in H₂O, pH 4.4. In some forms, the column is washed again with 3-4 column volumes of 1% AcOH in H₂O.

Analytical HPLC can be carried out to assess the purity and homogeneity of peptides. An exemplary HPLC column for use in analytical HPLC is a PHENOMENEX® JUPITER® column. A step gradient can be used to separate the peptide composition. In some embodiments, the gradient is from 1%-40% MeCN vs 0.05% TFA in H₂O. The change in gradient can be achieved over 20 minutes using a flow rate of 1 ml/min. Peptides can be detected using UV detection at 215 nm.

Where the peptides or compositions thereof are required to be sterilized or otherwise processed for the removal of undesirable contaminants and/or micro-organisms, filtration can be used. Filtration can be achieved using any system or procedures known in the art. In some forms, filtration removes contaminants or prevents the growth or presence of microorganisms. Exemplary microorganisms and contaminants that can be removed include bacteria, cells, protozoa, viruses, fungi, and combinations thereof. In some forms, the step of filtration is carried out to remove aggregated or oligomerized peptides. For example, solutions of the peptides can be filtered to remove oligomers on the basis of size.

### III. Compositions

### A. Peptides

Glycosylated peptides (also referred to herein as “glycopeptides”) based on or derived from adiponectin, and compositions and formulations thereof are disclosed. For example, in some forms, the glycosylated peptides can have the sequence of a naturally occurring adiponectin, such as a mammalian adiponectin.

Preferably, the glycopeptides are chemically synthesized, for example, by the methods disclosed herein. As such, in some forms, the glycopeptides are not recombinant. In some forms, the glycopeptides are not purified or isolated from a host, such as cells, tissues, or biological fluids (e.g., serum or adipocytes), or animals.

Adiponectin (also known as Acrp30, AdipoQ, GBP-28, and apM1) is the most abundant peptide secreted by adipocytes, and adiponectin reduction plays a central role in obesity-related diseases, including insulin resistance/type 2 diabetes and cardiovascular disease. Adiponectin is a hormone secreted mainly, but not exclusively, by adipose tissue. Other cell and tissue types, including hematopoietic progenitors, osteoblasts, liver parenchyma cells, myocytes, epithelial cells, endothelial cells, and placental tissue can also produce this adipocytokine³⁷. Adiponectin circulates in high concentrations in healthy adults, accounting for 0.01% of total plasma protein and its plasma levels are a thousand times that of leptin³⁸. Circulating levels of adiponectin range between 2 and 30 μg/ml in humans and are generally higher in females than males³⁸.

Human adiponectin is encoded by the Adipo Q gene, which spans 17 kb on chromosome locus 3q27. The gene for human adiponectin contains three exons, with the start codon in exon 2 and stop codon in exon 3. This human chromosome 3q27 has been identified as a region carrying a susceptibility gene for type 2 diabetes and metabolic syndrome. Full-length human adiponectin contains 244 amino acid residues, including an N-terminal signal sequence (amino acids 1-18), a variable region (amino acids 19-41), followed by a collagenous domain containing 22 Gly-XY repeats (amino acids 42-107), and a C-terminal C1q-like globular domain (amino acids 108-244)³⁷. In contrast to humans, mouse adiponectin is 247 amino acids long.

Adiponectin is secreted from adipocytes into the bloodstream where it is present in three main forms: trimers (˜100 kDa), hexamers (˜200 kDa), and high molecular weight (˜400-600 kDa) multimers containing at multiple monomers^{37,38}. The monomeric form of adiponectin is undetectable in native conditions. Homotrimer, also known as low molecular weight (LMW), is a basic building block of oligomeric adiponectin. The interaction between the collagenous domains results in formation of highly ordered trimer, which is further stabilized by an intratrimer disulfide bond. The formation of a disulfide bond between two trimers leads to the formation of the hexameric form of adiponectin. This hexameric form serves as the building block for the HMM form, which contains 12-18 hexamers existing in a bouquet-like structure³⁷.

To create these forms, a number of post-translational modifications are required. For example, post-translational modifications, especially hydroxylation and subsequent glycosylation of several highly conserved lysine residues within the collagenous domain, are crucial for the formation of HMW oligomeric adiponectin, which is the major bioactive isoform contributing to its insulin-sensitizing and cardiovascular protective effects. Globular adiponectin, the globular C1q domain of adiponectin generated from full-length protein by proteolysis, is also biologically active. Sialic acids also modified adiponectin through O-linked glycosylation situated on threonine residues within the hypervariable region, which determines the half-life of adiponectin in the circulation by modulating its clearance from the bloodstream. In addition, succination of the highly conserved cysteine residues (Cys36) within the hypervariable region of adiponectin blocks adiponectin multimerization and may contribute to the decrease in plasma adiponectin in diabetes. Therefore, post-translational modifications of adiponectin are essential for efficient maturation, oligomerization, and secretion of adiponectin, and are also important for maintaining its stability in the circulation.

Adiponectin effects are mediated by adiponectin receptors, including the isoforms AdipoR1 and AdipoR2. AdipoR1 is a high affinity receptor for globular adiponectin and a low affinity receptor for full length adiponectin. It is expressed ubiquitously, but most abundantly, in skeletal muscle. On the other hand, AdipoR2 mainly recognizes full length adiponectin and is predominantly expressed in the liver. Another receptor for adiponectin is T-cadherin, which acts as a receptor for hexameric and HMW forms of adiponectin, but not for other forms.

Adiponectin has direct actions in liver, skeletal muscle, and the vasculature. Adiponectin exhibits anti-diabetic, anti-inflammatory, and anti-atherogenic effects, and it also functions as an insulin sensitizer. Adiponectin also plays a central role in energy homeostasis through its action in hypothalamus³⁷. Adiponectin plays an important role in fat metabolism, feeding behavior, insulin sensitivity and is a negative regulator of hematopoiesis and immune responses. Adiponectin has been shown to suppress the expression of a number of membrane-bound proteins involved in the infiltration of cells to sites of inflammation, thereby indicating that adiponectin may inhibit inflammation. Adiponectin levels are reduced in human disease states such as obesity and coronary artery disease. Serum levels of adiponectin correlate with insulin sensitivity, and additionally, polymorphisms in the adiponectin gene result in an increased risk of insulin resistance and type 2 diabetes. Taken together, it is believed that adiponectin plays a role in the pathogenesis of obesity-related type 2 diabetes.

In some forms, the disclosed glycopeptides can be characterized by their biological function or activity. For example, a disclosed glycopeptide can be an agonist of the site of action of adiponectin or can be capable of eliciting the same biological response as adiponectin. Thus, the disclosed glycopeptides can be referred to as adiponectin mimetics. An adiponectin mimetic may have the ability to bind to or interact with one or more adiponectin receptors (AdipoR1 and AdipoR2) or variants thereof.

In some forms, contact or exposure of a cell, in vitro or in vivo, to one or more disclosed glycopeptides can reduce cell proliferation or viability. In some forms, administration of one or more disclosed glycopeptides to a subject reduces cancer cell proliferation, viability, or metastasis, reduces tumor growth or tumor burden, reduces body weight or body fat mass, prevents gain of body weight or body fat mass, improves glucose tolerance, improves insulin sensitivity, reduces or inhibits gluconeogenesis (e.g., hepatic gluconeogenesis), reduces triglyceride or cholesterol content/levels (e.g., in the liver or serum), reduces or inhibits inflammation (e.g., in the liver), reduces the expression levels of one or more liver injury biomarkers (e.g., ALT, AST, TNFα, CCL2, LDLR, COL1, COL6, TBL, ALP, IL-6, and IL-10), improves immune cell development and function, or combinations thereof in the subject.

i. Exemplary Glycopeptides and Variants

Thus, the disclosed glycopeptides are preferably based on or derived from adiponectin. For example, the glycopeptides can show sequence similarity to any adiponectin protein sequence or a portion thereof. Suitable adiponectin proteins include, but are not limited to, mammalian adiponectin such as mouse, rat, cat, dog, pig, sheep, monkey, cow, horse, and human. Mammalian adiponectin amino acid sequences are known in the art and include, for example, the following sequences from the UniProt database, which are hereby incorporated by reference: mouse (UniProt ID No. Q60994), rat (UniProt ID No. Q8K3R4), cat (UniProt ID No. A4PB30), dog (UniProt ID No. Q76C76), pig (UniProt ID No. Q6PP07), sheep (UniProt ID No. X4ZFS1), macaque (UniProt ID Nos. B1Q3K7, Q95JD7), cow (UniProt ID No. Q3Y5Z3), and horse (UniProt ID No. F7DZE7). Thus, the glycopeptides can include an amino acid sequence having at least 50%, 55%, 60%, 65%, 70%, 75%, 80%, 85%, 90%, 95%, 96%, 97%, 98%, 99%, 99.5% or more sequence identity to a mammalian adiponectin, including any of the foregoing.

In preferred forms, the glycopeptides are based on or derived from human adiponectin. For example, in some forms, the glycopeptides include an amino acid sequence having at least 50%, 55%, 60%, 65%, 70%, 75%, 80%, 85%, 90%, 95%, 96%, 97%, 98%, 99%, 99.5% or more sequence identity to human adiponectin. Amino acid sequences of human adiponectin are known in the art. See, for example, UniProt ID No. Q15848, which provides the following amino acid sequence:

(SEQ ID NO: 1)
MLLLGAVLLLLALPGHDQETTTQGPGVLLPLPKGACTGWMAGIPGHPGH
NGAPGRDGRDGTPGEKGEKGDPGLIGPKGDIGETGVPGAEGPRGFPGIQ
GRKGEPGEGAYVYRSAFSVGLETYVTIPNMPIRFTKIFYNQQNHYDGST
GKFHCNIPGLYYFAYHITVYMKDVKVSLFKKDKAMLFTYDQYQENNVDQ
ASGSVLLHLEVGDQVWLQVYGEGERNGLYADNDNDSTFTGFLLYHDTN.

In some forms, amino acid residues 1-18 of SEQ ID NO:1 form the signal domain (bolded and italicized), residues 19-41 of SEQ ID NO:1 form the variable region, residues 42-107 of SEQ ID NO:1 form the collagenous domain (bolded), and residues 108-244 of SEQ ID NO:1 form the globular (C1q) domain (italicized).

Thus, the glycopeptides can include an amino acid sequence having at least 50%, 55%, 60%, 65%, 70%, 75%, 80%, 85%, 90%, 95%, 96%, 97%, 98%, 99%, 99.5%, or 100% sequence identity to SEQ ID NO:1 or a portion of SEQ ID NO:1. In some forms, a preferred glycopeptide is or includes the peptide having the amino acid sequence of SEQ ID NO:1 (referred to as full-length human adiponectin).

In some forms, the glycopeptide is or includes one or more domains of human adiponectin, such as one or more domains selected from the signal domain, the variable region, the collagenous domain, and the globular domain. In a preferred form, the glycopeptide is or includes the collagenous domain of human adiponectin. An exemplary amino acid sequence of a collagenous domain of human adiponectin is: GIPGHPGHNGAPGRDGRDGTPGEKGEKGDPGLIGPKGDIGETGVPGAEGPRGFPGIQGR KGEPGEG (SEQ ID NO:2). Thus, the glycopeptides can include an amino acid sequence having at least 50%, 55%, 60%, 65%, 70%, 75%, 80%, 85%, 90%, 95%, 96%, 97%, 98%, 99%, 99.5%, or 100% sequence identity to SEQ ID NO:2 or a portion of SEQ ID NO:2.

In some forms, the glycopeptide can include other domains of adiponectin, such as the variable region or portion thereof in combination with the collagenous domain or portion thereof. In some forms, the glycopeptide is or includes the following amino acid sequence: WMAGIPGHPGHNGAPGRDGRDGTPGEKGEKGDPGLIGPKGDIGETGVPGAEGPRGFPGI QGRKGEPGEG (SEQ ID NO:3). SEQ ID NO:3 contains the last three amino acids of the variable region and the collagenous domain of the human adiponectin sequence of SEQ ID NO:1. Thus, the glycopeptides can include an amino acid sequence having at least 50%, 55%, 60%, 65%, 70%, 75%, 80%, 85%, 90%, 95%, 96%, 97%, 98%, 99%, 99.5%, or 100% sequence identity to SEQ ID NO:3 or a portion of SEQ ID NO:3.

Suitable peptides also include variants of the disclosed glycopeptides, such as the peptides of SEQ ID NO:1, 2, or 3, and modifications thereof retaining the same functional activity. For example, suitable peptides can include one or more point mutations or substitutions (e.g., 1, 2, 3, 4, 5 or more mutations) at any amino acid residue of SEQ ID NO:1, 2 or 3. Amino acid substitutions include conservative amino acid substitutions, although non-conservative substitutions can also be used.

Examples of conservative amino acid substitutions include those in which the substitution is within one of the five following groups: 1) small aliphatic, nonpolar or slightly polar residues (Ala, Ser, Thr, Pro, Gly); 2) polar, negatively charged residues and their amides (Asp, Asn, Glu, Gln); polar, positively charged residues (His, Arg, Lys); large aliphatic, nonpolar residues (Met, Leu, Ile, Val, Cys); and large aromatic resides (Phe, Tyr, Trp). Examples of non-conservative amino acid substitutions are those where 1) a hydrophilic residue, e.g., seryl or threonyl, is substituted for (or by) a hydrophobic residue, e.g., leucyl, isoleucyl, phenylalanyl, valyl, or alanyl; 2) a cysteine or proline is substituted for (or by) any other residue; 3) a residue having an electropositive side chain, e.g., lysyl, arginyl, or histidyl, is substituted for (or by) an electronegative residue, e.g., glutamyl or aspartyl; or 4) a residue having a bulky side chain, e.g., phenylalanine, is substituted for (or by) a residue that does not have a side chain, e.g., glycine.

It is understood that substitutions at amino acid positions can be made using any amino acid or amino acid analog. For example, the substitutions can be made with any of the naturally occurring amino acids (e.g., alanine, aspartic acid, asparagine, arginine, cysteine, glycine, glutamic acid, glutamine, histidine, leucine, valine, isoleucine, lysine, methionine, proline, threonine, serine, phenylalanine, tryptophan, or tyrosine).

Alanine scanning of peptides is useful for identifying amino acids that can be modified without reducing functional properties of the overall glycopeptide.

The term “variant” refers to a polypeptide that differs from a reference polypeptide, but retains essential properties. Generally, differences are limited so that the sequences of the reference polypeptide and the variant are closely similar overall and, in many regions, identical, but in all cases retain the same functional activity or mechanism of action. A variant and reference polypeptide may differ in amino acid sequence by one or more modifications (e.g., substitutions, additions, and/or deletions). A substituted or inserted amino acid residue may or may not be one encoded by the genetic code. A variant of a polypeptide may be naturally occurring such as an allelic variant, or it may be a variant that is not known to occur naturally.

ii. Glycopeptide PTMs

In preferred forms, the disclosed adiponectin-derived glycopeptides include one or more post-translational modifications (PTMs). PTMs generally refer to the covalent attachment of chemical groups to a protein after its synthesis. These modifications include phosphorylation, glycosylation, ubiquitination, nitrosylation, methylation, acetylation, lipidation and proteolysis. PTMs are most often mediated by enzymatic activity. Enzymes mediating PTMs include kinases, phosphatases, transferases and ligases, which add or remove functional groups, proteins, lipids or sugars to or from amino acid side chains; and proteases, which cleave peptide bonds to remove specific sequences or regulatory subunits. PTMs are important because they regulate protein activity, localization, and interaction with other cellular molecules such as proteins, nucleic acids, lipids and cofactors.

In preferred forms, the glycopeptides are hydroxylated and/or glycosylated. Hydroxylation is an oxidation reaction in which carbon-hydrogen (C—H) bond oxidizes into carbon-hydroxyl (C—OH) bond. In biology, hydroxylation is mediated by enzymes called hydroxylases. Hydroxylation of proteins occurs on three residues, most commonly proline at either the 3- or 4-position, lysine at the 5-position, and asparagine at the 3-position. Hydroxyproline and 5-hydroxylysine residues are abundant in many proteins including collagen. The hydroxyproline and hydroxylysine residues play important roles in the water solubility as well as in the triple helical structure formation in collagen fibrils.

Glycosylation generally refers to covalently linking carbohydrates (also called glycans) to lipid or protein molecules. A vast number of naturally occurring sugars can be combined to create a variety of unique glycan structures on lipid and protein molecules that modulate their function. Multiple enzymatic site preferences, as well as the use of stereochemical a or 3 conjugations, create further diversity in where and how these sugars are linked to each other. Glycans serve a variety of structural and functional roles in membrane and secreted proteins, including protein folding and stability, cell-to-cell adhesion, and immunity.

Suitable sugar moieties that can be included in the disclosed glycopeptides include monosaccharides, disaccharides, and oligosaccharides, such as, but not limited to: fucose (Fuc), galactose (Gal), glucose (GIc), galactosamine (GaINAc), glucosamine (GIcNAc), mannose (Man), N-acetyl-lactosamine (lacNAc), and N₅N′-diacetyllactosediamine (lacdiNAc). These sugar moieties can attach to polypeptide back bones in several ways, including: (1) via an N-glycosidic bond to the R-group of an asparagine residue (N-glycosylation; N-linked glycans); (2) via an O-glycosidic bond to the R-group of serine, threonine, hydroxyproline, tyrosine or hydroxylysine (O-glycosylation; O-linked glycans); (3) via the R-group of tyrosine in C-linked mannose; (4) as a glycophosphatidylinositol anchor used to secure some proteins to cell membranes; (5) as a single monosaccharide attachment of GIcNAc to the R-group of serine or threonine; (6) attachment of a linear polysaccharide to serine, threonine or asparagine (proteoglycans); and (7) via a S-glycosidic bond to the R-group of cysteine.

In some forms, the glycopeptides include one or more residues that are hydroxylated, glycosylated, or both hydroxylated and glycosylated. In some forms, one or more proline, lysine, or asparagine residues, or combinations thereof, are hydroxylated. In some forms, one or more asparagine, serine, threonine, or tyrosine residues, or combinations thereof, are glycosylated.

In particular forms, the glycopeptides include one or more residues which are both hydroxylated and glycosylated. For example, hydroxyproline and hydroxylysine residues can be glycosylated. It is known that in collagen, for example, the hydroxylysine residues are often glycosylated through the hydroxyl groups. Thus, the disclosed glycopeptides can include one or more glycosylated hydroxyproline and/or hydroxylysine residues.

In some forms, the glycosylation is with a single sugar moiety. A single sugar moiety can be, for example, sialic acid, glucosyl, galactosyl, N-acetylgalactosyl, N-acetylglucosyl, sialyl Lewis X, and fucosyl. In some forms, glycosylation is with multiple sugar moieties. In some forms, the glycosylation is with any one or more of a glucosylgalactosyl moiety, a glucosylglucosyl moiety, a galactosylglucosyl moiety, or a galactosylgalactosyl moiety. In preferred forms, the glycopeptides are glycosylated with 2-O-α-D-glucopyranosyl-D-galactose.

In preferred forms, the glycopeptides are glycosylated at one or more lysine residues, for example one or more hydroxylysine residues. In particular forms, the glycopeptides are glycosylated at one or more 5-(2S,5R)-hydroxylysine residues.

Since the glycopeptides are based on adiponectin, the glycosylated residues can be described based on the relative position in adiponectin. In some forms, the glycopeptides are glycosylated at one or more lysine residues in the collagenous domain of adiponectin, such as, the collagenous domain of human adiponectin. In preferred forms, the glycopeptides are glycosylated at one or more hydroxylysine residues (e.g., 5-(2S,5R)-hydroxylysine) in the collagenous domain of human adiponectin. Preferably one or more of the lysine residues corresponding to lysine residues 65, 68, 77, and 101 of human adiponectin is glycosylated. Residues 65, 68, 77, and 101 of human adiponectin are in the collagenous domain.

An exemplary amino acid sequence of a collagenous domain of human adiponectin is: GIPGHPGHNGAPGRDGRDGTPGEKGEKGDPGLIGPKGDIGETGVPGAEGPRGFPGIQGR KGEPGEG (SEQ ID NO:2). In some forms, when the glycopeptide is or includes the amino acid sequence of SEQ ID NO:2 or a portion thereof, any one or more (e.g., 1, 2, 3, or 4) of the italicized lysines (corresponding to residues 65, 68, 77, and 101 of human adiponectin) are hydroxylated and/or glycosylated.

In preferred forms, the glycopeptides contain the following amino acid sequence: WMAGIPGHPGHNGAPGRDGRDGTPGEKGEKGDPGLIGPKGDIGETGVPGAEGPRGFPGI QGRKGEPGEG (SEQ ID NO:3). In some forms, when the glycopeptide is or includes the amino acid sequence of SEQ ID NO:3 or a portion thereof, any one or more (e.g., 1, 2, 3, or 4) of the italicized lysines (corresponding to residues 65, 68, 77, and 101 of human adiponectin) are hydroxylated and/or glycosylated.

In some forms, the disclosed glycopeptides are glycosylated at one lysine residue in the collagenous domain of human adiponectin. Preferably, the glycosylated lysine residue is 5-(2S,5R)-hydroxylysine. In some forms, a disclosed glycopeptide is glycosylated at lysine residue 65 of human adiponectin. In some forms, the glycopeptide is glycosylated at lysine residue 68 of human adiponectin. In some forms, the glycopeptide is glycosylated at lysine residue 77 of human adiponectin. In some forms, the glycopeptide is glycosylated at lysine residue 101 of human adiponectin.

In some forms, the disclosed glycopeptides are glycosylated at two lysine residues in the collagenous domain of human adiponectin. Preferably, the glycosylated lysine residues are 5-(2S,5R)-hydroxylysine residues. In some forms, a disclosed glycopeptide is glycosylated at lysine residues 65 and 68 of human adiponectin. In some forms, the glycopeptide is glycosylated at lysine residues 65 and 77 of human adiponectin. In some forms, the glycopeptide is glycosylated at lysine residues 65 and 101 of human adiponectin. In some forms, the glycopeptide is glycosylated at lysine residues 68 and 77 of human adiponectin. In some forms, the glycopeptide is glycosylated at lysine residues 68 and 101 of human adiponectin. In some forms, the glycopeptide is glycosylated at lysine residues 77 and 101 of human adiponectin.

In some forms, the disclosed glycopeptides are glycosylated at three lysine residues in the collagenous domain of human adiponectin. Preferably, the glycosylated lysine residues are 5-(2S,5R)-hydroxylysine residues. In some forms, a disclosed glycopeptide is glycosylated at lysine residues 65, 68, and 77 of human adiponectin. In some forms, the glycopeptide is glycosylated at lysine residues 65, 68, and 101 of human adiponectin. In some forms, the glycopeptide is glycosylated at lysine residues 65, 77, and 101 of human adiponectin. In some forms, the glycopeptide is glycosylated at lysine residues 68, 77, and 101 of human adiponectin.

In some forms, the disclosed glycopeptides are glycosylated at all four lysine residues in the collagenous domain of human adiponectin. Preferably, the glycosylated lysine residues are 5-(2S,5R)-hydroxylysine residues. For example, the glycopeptide can be glycosylated at lysine residues 65, 68, 77, and 101 of human adiponectin.

In any of the foregoing, the glycosylation is with any one or more of a glucosylgalactosyl moiety, a glucosylglucosyl moiety, a galactosylglucosyl moiety, or a galactosylgalactosyl moiety. Preferably, the glycosylation is with 2-O-α-D-glucopyranosyl-D-galactose.

Disclosed peptides that differ from one another by the glycosylation (or lack thereof) at lysine residues (e.g., residues 65, 68, 77, and 101) are sometimes referred to herein as “glycoisoforms” or “glycoforms.” Glycoforms include peptides or protein having a constant primary structure but differing at the level of secondary or tertiary structure or co- or post-translational modification such as different positions of glycosylation.

### B. Other Peptide Modifications

The disclosed glycopeptides may be modified in various ways. In some forms, the modification(s) may render the glycopeptides more stable (e.g., resistant to degradation in vivo) or confer other desirable characteristics as will be appreciated by one skilled in the art. Such modifications include, without limitation, chemical modification, N terminus modification, C terminus modification, peptide bond modification, backbone modifications, residue modification, D-amino acids, non-natural amino acids, or others. In some forms, one or more modifications may be used simultaneously. In preferred forms, the peptides are stabilized against proteolysis. For example, the stability and activity of peptides can be improved by protecting some of the peptide bonds with N-methylation or C-methylation. It is believed that amidation can also enhance the stability of the glycopeptides to peptidases.

Modifications to the glycopeptides generally should leave them functional. It is understood that there are numerous amino acid analogs which can be incorporated into the peptides. For example, there are numerous D amino acids or other non-natural amino acids which can be used. The opposite stereoisomers of naturally occurring glycopeptides are disclosed, as well as the stereo isomers of peptide analogs. Amino acid analogs and peptide analogs often have enhanced or desirable properties, such as, more economical production, greater chemical stability, enhanced pharmacological properties (half-life, absorption, potency, efficacy, etc.), altered specificity (e.g., a broad-spectrum of biological activities), reduced antigenicity, and others.

Either or both ends of a given linear peptide can be modified. For example, the glycopeptides can be acetylated and/or amidated. In some forms, the glycopeptides are acetylated at the N-terminus.

The glycopeptides may contain naturally occurring α-amino acid residues, non naturally occurring α-amino acid residues, and combinations thereof. The D-enantiomer (“D-α-amino acid”) of residues may also be used. Incorporation of artificial amino acids such as beta or gamma amino acids and those containing non-natural side chains, and/or other similar monomers such as hydroxyacids are also contemplated, with the effect that the corresponding component is peptide-like in this respect.

Non-naturally occurring amino acids are not found or have not been found in nature, but they can by synthesized and incorporated into a peptide chain. Non-limiting examples of suitable non-natural amino acids (in L- or D-configuration) are azidoalanine, azidohomoalanine, 2-amino-5-hexynoic acid, norleucine, azidonorleucine, L-a-aminobutyric acid, 3-(1-naphthyl)-alanine, 3-(2-naphthyl)-alanine, p-ethynyl-phenylalanine, m-ethynyl-phenylalanine, p-ethynyl-phenylalanine, p-bromophenylalanine, p-idiophenylalanine, p-azidophenylalanine, and 3-(6-chloroindolyl) alanin.

In some forms, peptide bonds (—CO—NH—) within the glycopeptide can be substituted, for example, by N-methylated bonds (—N(CH3)-CO—), ester bonds (—C(R)H—C—0-0-C(R)—N—), ketomethylen bonds (—CO—CH2-), CC-aza bonds (—NH—N(R)—CO—), wherein R is any alkyl, e.g., methyl, carba bonds (—CH2-NH—), hydroxyethylene bonds (—CH(OH)—CH2-), thioamide bonds (—CS—NH—), olefinic double bonds (—CH═CH—), retro amide bonds (—NH—CO—), peptide derivatives (—N(R)—CH2-CO—), wherein R is the normal side chain, naturally presented on the carbon atom. These modifications can occur at any of the bonds along the peptide chain and even at several (e.g., 2, 3, 4 or more) at the same time.

The peptides can be utilized in a linear form, although it will be appreciated that in cases where cyclization does not severely interfere with peptide characteristics, cyclic forms of the peptides can also be used. As used herein in reference to a peptide, the term “cyclic” means a structure including an intramolecular bond between two non-adjacent amino acids or amino acid analogues. The cyclization can be effected through a covalent or non-covalent bond. A preferred method of cyclization is through formation of a disulfide bond between the side-chains of non-adjacent amino acids or amino acid analogs. A peptide also can cyclize, for example, via a lactam bond, which can utilize a side-chain group of one amino acid or analog thereof to form a covalent attachment to the N-terminal amine of the amino-terminal residue. Cyclization additionally can be effected, for example, through the formation of a lysinonorleucine bond between lysine (Lys) and leucine (Leu) residues or a dityrosine bond between two tyrosine (Tyr) residues. The skilled person understands that these and other bonds can be included in a cyclic glycopeptide.

Peptidomimetics may optionally be used to inhibit degradation of the peptides by enzymatic or other degradative processes. The peptidomimetics can be produced by organic synthetic techniques. Non-limiting examples of suitable peptidomimetics include D amino acids of the corresponding L amino acids. D-amino acids can be used to generate more stable peptides, because D amino acids are not recognized by peptidases and such. Systematic substitution of one or more amino acids in a given sequence with a D-amino acid of the same type (e.g., D-lysine in place of L-lysine) can be used to generate more stable peptides as long as activity is preserved.

In some forms, the peptides can contain one or more of the following modifications: glycosylation, amidation, acetylation, acylation, alkylation, alkenylation, alkynylation, phosphorylation, sulphorization, hydroxylation, hydrogenation, cyclization, ADP-ribosylation, anchor formation, covalent attachment of a lipid or lipid derivative, methylation, myristylation, pegylation, prenylation, esterification, biotinylation, coupling of farnesyl or ubiquitination, a linker which allows for conjugation or functionalization of the peptide, or a combination thereof.

In some forms, when the glycopeptide is a linear molecule, it is possible to place various functional groups at various points on the linear molecule which are susceptible to or suitable for chemical modification. In some forms, the functional groups improve the activity of the peptide with regard to one or more characteristics, including but not limited to, stability, penetration (e.g., through cellular membranes and/or tissue barriers), tissue localization, efficacy, decreased clearance, decreased toxicity, improved selectivity, improved resistance to expulsion by cellular pumps, and the like. Non-limiting examples of suitable functional groups are described in Green and Wuts, “Protecting Groups in Organic Synthesis,” the teachings of which are incorporated herein by reference.

In some forms, the glycopeptides can be modified to include one or more albumin-binding molecules or moieties. Such albumin-binding molecules or moieties can provide altered pharmacodynamics of the glycopeptide, such as alteration of tissue uptake, penetration, or diffusion; enhanced efficacy; and increased half-life. For example, the serum half-life of a peptide can be increased by linking it to a serum albumin-binding moiety and administering the peptide to a subject. The resulting conjugate will associate with circulating serum albumin and will remain in the serum longer than if the peptide were administered in the absence of a serum albumin-binding moiety. Thus, in particular forms, albumin-binding molecules or moieties are used to increase the half-life and overall stability of a disclosed glycopeptide that is administered to or enters the circulatory system of a subject. The albumin-binding moiety can be covalently or non-covalently linked, coupled or associated to the glycopeptide at a site that keeps the albumin-binding site of the moiety intact and still capable of binding to a serum albumin, without compromising the desired prophylactic or therapeutic activity of the glycopeptide. Exemplary albumin-binding molecules or moieties that can be used include, without limitation, fatty acids and derivatives thereof, small molecules, peptides, and proteins. See Zorzi A., et al., Med Chem Comm., 10(7):1068-1081 (2019), which is hereby incorporated by reference in its entirety, and which provides a review of albumin-binding ligands and their use in extending the circulating half-life of therapeutics.

### C. Pharmaceutical Formulations

Compositions and formulations of the disclosed glycopeptides are provided. In some forms, the compositions or formulations include one or more copies of the same glycopeptide. In some forms, the compositions or formulations include one or more copies of different glycopeptides (e.g., 2 or more glycopeptides). For example, the compositions or formulations can include multiple copies of each of two or more (e.g., 2, 3, 4, 5, or more) different glycoforms of the disclosed peptides.

The pharmaceutical compositions include the disclosed glycopeptides in combination with one or more pharmaceutically acceptable carriers and/or excipients that are considered safe and effective and can be administered to an individual without causing undesirable biological side effects or unwanted interactions. The carrier is all components present in the pharmaceutical formulation other than the active ingredient or ingredients. Suitable carriers, diluents and their formulations are described in Remington: The Science and Practice of Pharmacy (19th ed.) ed. A. R. Gennaro, Mack Publishing Company, Easton, P A 1995. Examples of pharmaceutically acceptable carriers include, but are not limited to, saline, Ringer's solution and dextrose solution. It will be apparent to those persons skilled in the art that certain carriers can be more preferable depending upon, for instance, the route of administration and concentration of composition being administered. Those of skill in the art can readily determine the various parameters for preparing and formulating the compositions without resort to undue experimentation.

Pharmaceutical compositions may be formulated in a conventional manner using one or more physiologically acceptable carriers including excipients and auxiliaries which facilitate processing of the active compounds into preparations which can be used pharmaceutically. The compositions may be administered in combination with one or more physiologically or pharmaceutically acceptable carriers, thickening agents, co-solvents, adhesives, antioxidants, buffers, viscosity and absorption enhancing agents and agents capable of adjusting osmolarity of the formulation. Proper formulation is dependent upon the route of administration chosen. If desired, the compositions may also contain minor amounts of nontoxic auxiliary substances such as wetting or emulsifying agents, dyes, pH buffering agents, or preservatives.

The formulations can contain a preservative to prevent the growth of microorganisms. Suitable preservatives include, but are not limited to, parabens, chlorobutanol, phenol, sorbic acid, and thimerosal. The formulations can also contain an antioxidant to prevent degradation of the active agent(s).

Pharmaceutical compositions of the glycopeptides may be for systemic or local administration. In some forms, the compositions can be formulated for administration by parenteral (e.g., intramuscular (IM), intraperitoneal (IP), intravenous (IV), intra-arterial, intrathecal, or subcutaneous injection (SC)), transmucosal (e.g., nasal, vaginal, pulmonary, or rectal), or enteral routes of administration. In some forms, the compositions are formulated for mucosal administration, such as through nasal, pulmonary, oral (e.g., sublingual, buccal), vaginal, or rectal mucosa delivery.

i. Parenteral Formulations

The compositions may be formulated for parenteral administration, such as by injection, e.g., by bolus injection or continuous infusion. Parenteral administration can include administration to a subject intravenously, intradermally, intraarterially, intraperitoneally, intracranially, intraarticularly, intraprostatically, intrapleurally, intratracheally, intravitreally, intratumorally, intramuscularly, subcutaneously, subconjunctivally, intravesicularly, intrapericardially, intraumbilically, by injection, and by infusion.

For injection, the glycopeptides and compositions thereof may be formulated in aqueous solutions, preferably in physiologically compatible buffers such as Hanks's solution, Ringer's solution, or physiological saline buffer. Parenteral formulations also include solid forms suitable for using to prepare solutions or suspensions upon the addition of a reconstitution medium prior to injection. Formulations for injection may be presented in unit dosage form, e.g., in ampules or in multi-dose containers, optionally with an added preservative.

The compositions may take such forms as sterile aqueous or non-aqueous solutions, suspensions and emulsions, which can be isotonic with the blood of the subject in certain forms. Examples of non-aqueous solvents are polypropylene glycol, polyethylene glycol, vegetable oil such as olive oil, sesame oil, coconut oil, arachis oil, peanut oil, mineral oil, injectable organic esters such as ethyl oleate, or fixed oils including synthetic mono or di-glycerides. Aqueous carriers include water, alcoholic/aqueous solutions, emulsions or suspensions, including saline and buffered media. Parenteral vehicles include sodium chloride solution, 1,3-butandiol, Ringer's dextrose, dextrose and sodium chloride, lactated Ringer's or fixed oils. Intravenous vehicles include fluid and nutrient replenishers, and electrolyte replenishers (such as those based on Ringer's dextrose). The compositions may be in solution, emulsions, or suspension (for example, incorporated into particles or liposomes). Typically, an appropriate amount of a pharmaceutically acceptable salt is used in the formulation to render the formulation isotonic. Trehalose, typically in the amount of 1-5%, may be added to the pharmaceutical compositions.

The pH of the solution can be preferably from about 5 to about 8, and more preferably from about 7 to about 7.5. Suitable buffers include, but are not limited to, phosphate buffers, acetate buffers, and citrate buffers.

Sterile injectable solutions can be prepared by incorporating the glycopeptides in the required amount in the appropriate solvent or dispersion medium with one or more of the excipients, as required, followed by filtered sterilization. Generally, dispersions are prepared by incorporating the various sterilized active ingredients into a sterile vehicle which contains the basic dispersion medium and the required other ingredients from those listed above. In the case of sterile powders for the preparation of sterile injectable solutions, the preferred methods of preparation are vacuum-drying and freeze-drying techniques which yield a powder of the active ingredient plus any additional desired ingredient from a previously sterile-filtered solution thereof. The powders can be prepared in such a manner that the particles are porous in nature, which can increase dissolution of the particles. Methods for making porous particles are well known in the art.

For parenteral administration, active agents such as the glycopeptides, can be incorporated into microparticles, nanoparticles, or combinations thereof that provide controlled release of the active agents. For example, active agent(s) can be incorporated into polymeric microparticles, which provide controlled release of the agent(s). Release of the agent(s) is controlled by diffusion of the agent(s) out of the microparticles and/or degradation of the polymeric particles by hydrolysis and/or enzymatic degradation. Suitable polymers include ethylcellulose and other natural or synthetic cellulose derivatives.

ii. Enteral Formulations

Enteral administration (e.g., oral, sublingual) may be used, e.g., when the glycopeptides are stable enough to withstand the harsh proteolytic environment of the gastrointestinal tract. Suitable oral dosage forms include tablets, capsules, solutions, suspensions, syrups, and lozenges. Tablets can be made using compression or molding techniques well known in the art. Gelatin or non-gelatin capsules can prepared as hard or soft capsule shells, which can encapsulate liquid, solid, and semi-solid fill materials, using techniques well known in the art. In forms where the formulation is for oral administration involving transit through the gastrointestinal tract, the formulation is preferably coated to protect the peptide from gastrointestinal enzymes.

In some forms, the compositions can be formulated readily by combining the glycopeptide compositions with pharmaceutically acceptable carriers well known in the art. Carriers include, without limitation, diluents, preservatives, binders, lubricants, disintegrators, swelling agents, fillers, stabilizers, and combinations thereof. Carriers also include all components of the coating composition, which can include plasticizers, pigments, colorants, stabilizing agents, and glidants. Such carriers enable the compositions to be formulated as tablets, pills, dragees, capsules, liquids, gels, syrups, slurries, suspensions and the like, for oral ingestion by a subject to be treated.

Pharmacological preparations for oral use can made with the use of a solid excipient, optionally grinding the resulting mixture, and processing the mixture of granules, after adding suitable auxiliaries, if desired, to obtain tablets. Suitable excipients include fillers such as sugars, including lactose, sucrose, mannitol, or sorbitol; cellulose preparations such as, for example, maize starch, wheat starch, rice starch, potato starch, gelatin, gum tragacanth, methyl cellulose, hydroxypropylmethyl-cellulose, sodium carboxymethylcellulose, and/or polyvinylpyrrolidone (PVP). If desired, disintegrating agents may be added, such as the cross-linked polyvinyl pyrrolidone, agar, or alginic acid or a salt thereof such as sodium alginate.

Oral dosage forms, such as capsules, tablets, solutions, and suspensions, can for formulated for controlled release. For example, active agents (e.g., glycopeptides) can be formulated into nanoparticles, microparticles, and combinations thereof, and encapsulated in a soft or hard gelatin or non-gelatin capsule or dispersed in a dispersing medium to form an oral suspension or syrup. The particles can be formed of the active agents (e.g., glycopeptides) and a controlled release polymer or matrix. Alternatively, the active agents (e.g., glycopeptides) can be coated with one or more controlled release coatings prior to incorporation into the finished dosage form.

In some forms, one or more active agents are dispersed in a matrix material, which gels or emulsifies upon contact with an aqueous medium, such as physiological fluids. In the case of gels, the matrix swells entrapping the active agents, which are released slowly over time by diffusion and/or degradation of the matrix material. Such matrices can be formulated as tablets or as fill materials for hard and soft capsules.

In still some forms, one or more active agents are formulated into a solid oral dosage form, such as a tablet or capsule, and the solid dosage form is coated with one or more controlled release coatings, such as delayed release coatings or extended-release coatings. The coating or coatings can also contain one or more active agents.

### IV. Methods of Use

Disclosed herein are various methods related to the disclosed glycopeptides and compositions and their use. The glycopeptides and compositions thereof can be used in therapeutic, prophylactic, and/or diagnostic applications. For example, adiponectin is known as an anti-inflammatory, antioxidative, anti-atherogenic, proapoptotic, and antiproliferative adipokine and it has insulin sensitizing effect. Thus, the disclosed adiponectin-based glycopeptide formulations can be used to exert anti-inflammatory, antioxidative, anti-atherogenic, proapoptotic, antiproliferative, or insulin sensitizing effects, or combinations thereof.

Methods of treating a disease or disorder, or one or more symptoms thereof are provided. In preferred forms, the glycopeptide compositions can be used to treat, prevent or manage a disease or condition, in a subject. The glycopeptide compositions can also be used to reduce, manage, delay or prevent one or more symptoms of a disease, disorder, or condition, in a subject in need thereof. Thus, disclosed are methods of treating one or more diseases or disorders associated with adiponectin regulation, or reduced or even abolished adiponectin expression or levels.

Suitable diseases or disorders to be treated include, but are not limited to, cancer, inflammation, autoimmune diseases, neurological degeneration, hyperglycemia, insulin resistance, metabolic syndromes associated with insulin resistance, Type 1 diabetes, Type 2 diabetes, obesity, metabolic syndrome, hypertension, atherosclerosis, steatohepatitis, coronary heart disease, ischemic heart disease, polycystic ovary syndrome, fatty liver disease, cardiovascular disease, endothelial dysfunction, cellular infiltration to sites of inflammation, or other diseased states associated with adiponectin or obesity. Metabolic syndrome, also known as syndrome X or insulin resistance syndrome, is a collection of obesity-associated disorders that includes dyslipidemia (triglyceride (TG)>150 mg/dl, high-density lipoprotein (HDL) cholesterol (<40 mg/dl in males and <50 in females), impaired fasting glucose (fasting glucose ≥100) and visceral adiposity (waist circumference >102 cm in men and >88 cm in woman). Also, this syndrome is associated with prothrombotic state, inflammation, oxidative stress, elevated risk of developing cardiovascular disease (CVD) like atherosclerosis and type 2 diabetes (T2D).

In particular forms, the disclosed glycopeptide compositions are used in methods of treating cancer, such as carcinomas, sarcomas, lymphomas and leukemias. The described compositions and methods are useful for treating, or alleviating subjects having benign or malignant tumors by delaying or inhibiting the growth/proliferation or viability of tumor cells in a subject, reducing the number, growth or size of tumors, inhibiting or reducing metastasis of the tumor, and/or inhibiting or reducing symptoms associated with tumor development or growth. The types of cancer that can be treated with the provided compositions and methods include, but are not limited to, cancers such as blood/hematological cancer, myeloma, adenocarcinomas and sarcomas, of bone, bladder, brain, breast, cervical, colorectal, esophageal, kidney, liver, lung, nasopharangeal, pancreatic, prostate, skin, stomach, thyroid, ovarian, testicular, and uterine. Preferably, the cancer to be treated is a lung cancer such as small cell or non-small cell lung cancer (including adenocarcinoma, squamous cell carcinoma, and large cell carcinoma) or breast cancer. In some forms, the compositions are used to treat multiple cancer types concurrently. The compositions can also be used to treat metastases or tumors at multiple locations.

In some forms, a method of treating a disease or disorder includes administering to a subject an effective amount of pharmaceutical formulation containing any of the disclosed glycopeptides or compositions thereof. For example, a subject can be administered an effective amount of a composition containing glycopeptides and a pharmaceutically acceptable carrier.

In preferred forms, pharmaceutical formulations containing one or more of the disclosed glycopeptides can be administered to a subject to reduce cancer cell proliferation or viability, reduce tumor growth or tumor burden, reduce body weight or body fat mass, prevent gain of body weight or body fat mass, improve glucose tolerance, improve insulin sensitivity, reduce or inhibit gluconeogenesis (e.g., hepatic gluconeogenesis), reduce lipid (e.g., triglyceride or cholesterol) content/levels (e.g., in the liver or serum), reduce or inhibit inflammation (e.g., in the liver), reduce the expression levels of one or more liver injury biomarkers (e.g., ALT, AST, TNFα, CCL2, LDLR, COL1, COL6, TBL, ALP, IL-6, and IL-10) e.g., in the liver, or combinations thereof in the subject.

In some forms, administation of the glycopeptide formulations reduces cancer cell proliferation or viability and/or reduces tumor burden in a subject. These effects on cell proliferation or viability or tumor burden can be direct (e.g., not mediated through an intermediate) or indirect (e.g., effects are mediated through one or more intermediates, e.g., a cell or signaling molecule). In some forms, the glycopeptide formulations may lead to direct, and/or indirect reduction of tumor cell proliferation by 5%, 10%, 20%, 30%, 40%, 50%, 60%, 70%, 80%, 90%, or more than 90%. In some forms, the glycopeptide formulations may lead to direct and/or indirect reduction of cancer cell viability by 5%, 10%, 20%, 30%, 40%, 50%, 60%, 70%, 80%, 90%, or more than 90%. In some forms, the glycopeptide formulations may lead to direct, and/or indirect reduction in tumor burden by 5%, 10%, 20%, 30%, 40%, 50%, 60%, 70%, 80%, 90%, or more than 90%. It is to be understood that the aforementioned reductions are relative to a control, which need not be stated. One of ordinary skill in the art can determine the appropriate control. For example, in some forms, reduction is relative to a state prior to administration of the glycopeptide compositions. In some forms, reduction is relative to a subject who is not administered the compositions.

The subject can be treated with the disclosed peptides and/or other active agents by administering an effective amount of the peptide and/or other active agents to the subject, enterally, by pulmonary or nasal administration, or parenterally (intravenously, intradermally, intraarterially, intraperitoneally, intracranially, intraarticularly, intraprostatically, intrapleurally, intratracheally, intravitreally, intratumorally, intramuscularly, subcutaneously, subconjunctivally, intravesicularly, intrapericardially, intraumbilically, by injection, and by infusion.

Preferably, the subject is a human.

### A. Effective Amounts

Typically, the methods involve administering an effective amount of the pharmaceutical compositions. For example, in some forms, the compositions are administered to a subject in an effective amount for treatment and/or prevention of a disease, disorder or condition e.g., caused by reduced adiponectin expression and/or activity.

The effective amount can be a dosage sufficient to treat, inhibit, or alleviate one or more symptoms of a disease, disorder or condition being treated or to otherwise provide a desired pharmacologic and/or physiologic effect. The effective amount of the glycopeptide compositions will vary from subject to subject, and can depend on the species, age, weight and general condition of the subject, the severity of the disorder being treated, and the mode of administration. Thus, it is not possible to specify an exact amount for every therapeutic composition. However, an appropriate amount can be determined by one of ordinary skill in the art using only routine experimentation given the teachings herein. For example, effective dosages and schedules for administering the therapeutics may be determined empirically, and making such determinations is within the skill in the art. The dosage ranges for the administration of the compositions are those large enough to effect one or more desired responses.

As further studies are conducted, information will emerge regarding appropriate dosage levels for treatment of various conditions in various patients, and the ordinary skilled worker, considering the therapeutic context, age, and general health of the recipient, will be able to ascertain proper dosing. The selected dosage can depend upon the age, condition, and sex of the subject, the desired therapeutic effect, on the route of administration, and on the duration of the treatment desired. The dosage should not be so large as to cause adverse side effects, such as unwanted cross-reactions, anaphylactic reactions, and the like. The dosage can be adjusted by the individual physician in the event of any counter-indications. It will also be appreciated that the effective dosage of the composition used for treatment may increase or decrease over the course of a particular treatment. Changes in dosage may result and become apparent from the results of diagnostic assays.

In some forms, the amount of the glycopeptide composition administered is effective to reduce cancer cell proliferation or viability, reduce tumor growth or tumor burden, reduce body weight or body fat mass, prevent gain of body weight or body fat mass, improve glucose tolerance, improve insulin sensitivity, reduce or inhibit gluconeogenesis (e.g., hepatic gluconeogenesis), reduce lipid (e.g., triglyceride or cholesterol) content/levels (e.g., in the liver or serum), reduce or inhibit local or systemic inflammation (e.g., in the liver), reduce the expression levels of one or more liver injury biomarkers (e.g., ALT, AST, TNFα, CCL2, LDLR, COL1, COL6, TBL, ALP, IL-6, and IL-10) e.g., in the liver, or combinations thereof in the subject.

In some forms, pharmaceutical formulations containing one or more of the glycopeptides is administered to a subject in an effective amount to induce or increase the production of anti-inflammatory cytokines, such as interleukin 10 (IL-10), interleukin-1 receptor antagonist (IL-1RA).

In some forms, pharmaceutical formulations containing one or more of the glycopeptides is administered to an obese subject in an effective amount to induce weight loss. In some forms, pharmaceutical formulations containing one or more of the glycopeptides is administered to an obese subject in an effective amount to decrease body mass by at least 10% (e.g., at least 15% or 20%). In some forms, pharmaceutical formulations containing one or more of the glycopeptides is administered to an obese subject in an effective amount to decrease body fat by at least 10% (e.g., at least 15% or 20%).

In some forms, pharmaceutical formulations containing one or more of the glycopeptides is administered to a subject in an effective amount to improve glucose homeostasis. In some forms, pharmaceutical formulations containing one or more of the glycopeptides is administered to a subject in an effective amount to reduce average fasting plasma blood glucose e.g., by at least 10% (e.g., at least 15% or 20%). In some forms, pharmaceutical formulations containing one or more of the glycopeptides is administered to a subject in an effective amount to reduce fasting plasma glucose levels to less than about 180 mg/dL (e.g., less than about 160 mg/dL, or less than about 140 mg/dL).

In some forms, pharmaceutical formulations containing one or more of the glycopeptides is administered to a subject in an effective amount to induce or increase activity or signaling of one or more adiponectin receptors (e.g., AdipoR1, AdipoR2, and/or T-cadherin). For example, in some forms, pharmaceutical formulations containing one or more of the glycopeptides is administered to a subject in an effective amount to activate or increase AMP-activated protein kinase (AMPK), PPAR-α, p38 mitogen-activated protein kinase (MAPK) signaling pathways (e.g., in the liver, muscle, and/or adipocytes) and/or semaphorin-4D (SEMA4D) (also known as CD100).

The glycopeptide compositions can be administered to a subject at a suitable dose, such as from about 1 μg/kg to about 20 mg/kg, for example, from about 1 mg/kg to about 10 mg/kg.

In some forms, an effective amount of the glycopeptide composition or the effects thereof can be compared to a control. Suitable controls are known in the art. A typical control is a comparison of a condition or symptom of a subject prior to and after administration of the composition. For example, the effect of the composition on a particular symptom, pharmacologic, or physiologic indicator (including those mentioned above and elsewhere herein) can be compared to an untreated subject, or the condition of the subject prior to treatment. In some form, the symptom, pharmacologic, or physiologic indicator is measured in a subject prior to treatment, and again one or more times after treatment is initiated. The condition or symptom can be a biochemical, molecular, physiological, or pathological readout. In some forms, the control is a matched subject that is administered a different agent or that does not receive any treatment. In some forms, the control is a reference level, or average determined based on measuring the symptom, pharmacologic, or physiologic indicator in one or more subjects that do not have the disease or condition to be treated (e.g., healthy subjects). In some forms, the effective amount or effect of the compositions is compared to other art recognized treatments for the disease or condition to be treated or prevented.

### B. Dosing Regimens

Dosages and timing of administration can vary. Guidance can be found in the literature for appropriate dosages for given classes of pharmaceutical products. Optimal dosing schedules can be calculated from measurements of drug accumulation in the body of the subject or patient. Persons of ordinary skill can determine optimum dosages, dosing methodologies, and repetition rates. Optimum dosages may vary depending on the relative potency of individual therapeutics and can generally be estimated based on EC50s found to be effective in in vitro and in vivo animal models.

Treatment can be continued for an amount of time sufficient to achieve one or more desired goals (e.g., therapeutic or prophylactic goals). Treatment can be continued for a desired period of time, and the progression of treatment can be monitored using any suitable means known in the art. In some forms, administration is carried out every day of treatment, or every week, or every fraction of a week. In some forms, treatment regimens are carried out over the course of up to two, three, four or five days, weeks, or months, or for up to 6 months, or for more than 6 months, for example, up to one or two years.

The compositions can be administered during a period during, or after onset of disease symptoms, or any combination of periods during or after diagnosis of one or more disease symptoms. For example, the subject can be administered one or more doses of the composition every 1, 2, 3, 4, 5, 6, 7, 14, 21, 28, 35, or 48 days after the onset or diagnosis of disease symptoms. In some forms, multiple doses of the compositions are administered before an improvement in disease condition is evident. For example, in some forms, the subject receives the composition, over a period of 1, 2, 3, 4, 5, 6 7, 14, 21, 28, 35, or 48 days or weeks before an improvement in the disease or condition is evident.

In some forms, the subject is a patient in intensive care. In the intensive care setting, the glycopeptide compositions can be administered over the course of one or more hours, for example, as a rescue therapy or salvage therapy. In some forms, the glycopeptide compositions can be administered as a preventative.

The composition can be administered hourly, daily, weekly, or monthly, one or more times, as required. In a particular form, the compositions are delivered to a subject or patient via intravenous infusion over the course of one or more hours. In some forms, the composition is administered or applied for a time of from about 30 seconds to about 30 minutes, for example about 30 seconds, about 1, 2, 3, 4, 5, 6, 7, 8, 9, 10, 11, 12, 13, 14, 15, 16, 17, 18, 19, 20, 25, or 30 minutes. In some forms, the composition is administered or applied one or more times per day, e.g., 1, 2, 3 or more times per day.

### C. Combination Therapy

In some forms, the disclosed glycopeptide composition is administered in combination with one or more additional active agents. Such combination therapies can include administration of the active agents together in the same admixture, or in separate admixtures. Therefore, in some forms, the pharmaceutical composition includes two, three, or more active agents. Such formulations typically include an effective amount of one or more disclosed glycopeptides or a pharmaceutically acceptable salt thereof.

The term “combination” or “combined” is used to refer to either concomitant, simultaneous, or sequential administration of two or more agents. Therefore, the combinations can be administered either concomitantly (e.g., as an admixture), separately but simultaneously (e.g., via separate intravenous lines into the same subject; one agent is given orally while the other agent is given by infusion or injection, etc.,), or sequentially (e.g., one of the compounds or agents is given first followed by the second).

Any suitable agent can be used in the combination therapy. Exemplary additional active agents include anti-inflammatory agents, analgesics, and chemotherapeutics. In some forms, additional active agents include leptin, insulin or an analog thereof, and/or celastrol.

In some forms, the additional active agent is an agent conventionally used in the treatment of the disease or disorder being treated. In certain forms, a subject suffering from diabetes is co-administered one or more therapies for diabetes in combination with a disclosed glycopeptide composition. For example, in the case of diabetes, insulin or an analog thereof is a suitable additional active agent.

In certain forms, a subject suffering from cancer is co-administered one or more anticancer therapies in combination with a disclosed glycopeptide composition. Exemplary additional anticancer therapies or agents include surgery, radiation therapy, gefitinib, erlotinib, cisplatin, 5-fluorouracil, tegafur, raltitrexed, cytosine arabinoside, hydroxyurea, adriamycin, bleomycin, daunomycin, mitomycin-C, dactinomycin and mithramycin, vincristine, vinblastine, vindesine, vinorelbine, etoposide, teniposide, topotecan, camptothecin bortezomib anegrilide, tamoxifen, toremifene, raloxifene, droloxifene, iodoxyfene fulvestrant, bicalutamide, flutamide, nilutamide, cyproterone, goserelin, leuprorelin, buserelin, megestrol, anastrozole, letrozole, vorazole, exemestane, finasteride, marimastat, trastuzumab (HERCEPTIN®), bevacizumab (AVASTIN®), gemtuzumab (MYLOTARG®), panitumumab (VECTIBIX®) or edrecolomab (PANOREX®), tyrosine kinase inhibitor, such as sorafenib (NEXAVAR®) or sunitinib (SUTENT®), cetuximab, dasatinib, imatinib, combretastatin, thalidomide, and/or lenalidomide, alkylating agents; alkyl sulfonates; aziridines, such as Thiotepa; ethyleneimines; anti-metabolites; folic acid-analogues, such as methotrexate (FARMITREXAT®, LANTAREL®, METEX®, MTX HEXAL®); purine analogues, platinum-compounds, such as cisplatin or carboplatin; as well as derivates, tautomers and pharmaceutically active salts of the aforementioned compounds.

In some forms, the one or more additional active agents is one or more other targeted cancer therapies and/or immune-checkpoint blockage agents. In some forms, the additional active agent is an anti-PD-1 or anti-PD-L1 antibody. Anti-PD-L1 antibodies and antigen-binding fragments thereof suitable for use are known in the art and include atezolizumab, avelumab, durvalumab, pembrolizumab, nivolumab and antibodies disclosed in U.S. Pat. No. 9,624,298, which is hereby incorporated by reference in its entirety. In some forms, the additional active agent is an anti-CTLA-4 antibody (e.g., Ipilimumab and Tremelimumab).

In some forms, the combination of two or more active agents achieves a result greater than when the individual agents are administered alone or in isolation. For example, in some forms, the result achieved by the combination is partially or completely additive of the results achieved by the individual agents alone. In some forms, the result achieved by the combination is more than additive of the results achieved by the individual agents alone.

A treatment regimen of a combination therapy can include one or multiple administrations of each active agent. In certain forms, the two or more agents are administered simultaneously in the same or different pharmaceutical compositions. In some forms, two or more active agents are administered sequentially, typically, in two or more different pharmaceutical compositions. The different active agents be administered hours or days apart.

Dosage regimens or cycles of the agents can be completely or partially overlapping or can be sequential. In some forms, all such administration(s) of one agent occurs before or after administration of the second and/or subsequent agent. Alternatively, administration of one or more doses of the one or more agents can be temporally staggered.

An effective amount of each of the agents can be administered as a single unit dosage (e.g., as dosage unit), or sub-therapeutic doses that are administered over a finite time interval. Such unit doses can be administered on a daily basis for a finite time period, such as up to 3 days, or up to 5 days, or up to 7 days, or up to 10 days, or up to 15 days, or up to 20 days, or up to 25 days.

### V. Kits

The disclosed reagents, materials, and compositions as well as other materials can be packaged together in any suitable combination as a kit useful for performing, or aiding in the performance of, the disclosed methods. It is useful if the components in a given kit are designed and adapted for use together in the disclosed method.

Dosage units including the disclosed compositions, for example, in a pharmaceutically acceptable carrier for shipping, storage and/or administration are provided. Components of the kit may be packaged individually and can be sterile. The kits typically include a container containing one or more of the active agents (e.g., the disclosed glycopeptides) described herein. In certain forms, the active agent(s) can be provided in a unit dosage formulation (e.g., suppository, tablet, caplet, patch, etc.) and/or may be optionally combined with one or more pharmaceutically acceptable excipients.

A kit with one or more compositions for administration to a subject, may include a pre-measured dosage of the composition in a sterile needle, ampule, tube, container, or other suitable vessel. In some forms, the active agents can be supplied alone (e.g., lyophilized).

In some forms, a pharmaceutically acceptable carrier containing an effective amount of the composition is shipped and stored in a sterile vial. The sterile vial may contain enough composition for one or more doses. The composition may be shipped and stored in a volume suitable for administration or may be provided in a concentration that is diluted prior to administration.

In some forms, a pharmaceutically acceptable carrier containing drug can be shipped and stored in a syringe. Kits containing syringes of various capacities or vessels with deformable sides (e.g., plastic vessels or plastic-sided vessels) that can be squeezed to force a liquid composition out of an orifice are provided. The size and design of the syringe will depend on the route of administration. Any of the kits can include instructions for use. The instructions can be in the form of a publication, a recording, a diagram, or any other medium of expression which can be used to communicate the usefulness of the compositions and methods of the kit. For example, the instructional material may provide instructions for methods using the kit components, such as reconstituting dried powder formulations, performing dilutions, administration of injectable doses, and the like.

### EXAMPLES

The following examples are put forth so as to provide those of ordinary skill in the art with a complete disclosure and description of how the compounds, compositions, articles, devices and/or methods claimed herein are made and evaluated and are intended to be purely exemplary and are not intended to limit the disclosure.

### Example 1: Synthesis of a Hydroxylysine Building Block

### Material and Methods

### General Information

All commercial materials (Aldrich, Chemimpex, Fluka and GL Biochem) were used without further purification. All solvents were reagent grade or HPLC grade (RCI or DUKSAN). Dry dichloromethane (CH₂Cl₂) was distilled from calcium hydride (CaH₂). All reversed-phase (RP) high-performance liquid chromatography (HPLC) separations involved a mobile phase of 0.1% trifluoroacetic acid (TFA) (v/v) in acetonitrile (CH₃CN)/0.1% TFA (v/v) in water (H₂O) were performed with a Waters HPLC system equipped with a photodiode array detector (Waters 2996) using a Vydac 214TPTM C4 column (5 μm, 300 Å, 4.6×250 mm) at a flow rate of 0.6 mL/min for analytical HPLC and Vydac 214TPTM C4 column (10 μm, 300 Å, 22×250 mm) or Vydac 218TPTM C18 column (10 μm, 300 Å, 22×250 mm) at a flow rate of 10 mL/min for preparative HPLC. Low-resolution mass spectral (MS) analyses were performed with a Waters 3100 mass spectrometer using electrospray ionization (ESI, in positive mode unless otherwise specified). The results were analyzed with Waters Empower software. Calculated masses were based upon the most abundant isotope of a given ion. Analytical TLC was performed on E. Merck silica gel 60 F254 plates and visualized under UV light (254 nm) or by staining with ninhydrin or 5% sulfuric acid in ethanol. Silica flash column chromatography was performed on E. Merck 230-400 mesh silica gel 60. 1H and 13C nuclear magnetic resonance (NMR) spectra were recorded at 298 K on Bruker Avance DRX 300 FT-NMR Spectrometer at 75 MHz for 13C NMR or Bruker Avance DRX 400 FT-NMR spectrometer at 400 MHz for 1H NMR and 100 MHz for 13C NMR or Bruker Avance DRX 600 FT-NMR spectrometer at 150 MHz for 13C NMR. Chemical shifts were reported in parts per million (ppm) and were referenced to solvent residual signals: CDCl3 (δ 7.26 [1H]). 1H NMR data was reported as chemical shift (δ), relative integral, multiplicity (s=singlet, d=doublet, t=triplet, dd=doublet of doublets, td=triplet of doublets), coupling constant (J Hz). UPLC-MS=Ultra Performance Liquid Chromatography mass-spectrometry; PG=protecting groups; SAL=salicylaldehyde; DMF=dimethylformamide; TIPS=triisopropylsilane; Mbn=4-methyl benzyl. For glycosylated 5-hydroxylysine building block coupling, double coupling is required for sufficient conversion (1.2 equiv, 0.1M, 6 h, twice).

### Synthesis of L-α-vinylglycine

### Compound 1.1

KHCO₃ (13.5 g, 134.60 mmol, 2.5 equiv.) was slowly added to the mixture of Fmoc-Met-OH 1 (20.0 g, 53.80 mmol, 1.0 equiv.), BnBr (13.7 g, 80.70 mmol, 1.5 equiv.) in anhydrous DMF (50 mL). The reaction mixture was stirred at room temperature for 4 h. Till the full conversion of the acid 1, the reaction mixture was diluted with EtOAc (500 mL) and washed sequentially with 1 N HCl (3×100 mL) and brine (100 mL). The organic phase was dried over Na2SO4 and concentrated under vacuum. Purification by silica gel chromatography (n-hexane/EtOAc=6:1) gave Fmoc-Met-OBn 1.1 (18.6 g, 75%) as a white solid.

¹H NMR (400 MHz, CDCl3), δ=7.75 (2H, d, J=7.5 Hz), 7.57 (2H, d, J=7.5 Hz), 7.26-7.40 (9H, m), 5.46 (1H, d, J=8.0 Hz), 5.19 (2H, dd, J=8.0 Hz, 9.2 Hz), 5.05 (1H, dd, J=12.6 Hz), 4.41 (2H, d, J=6.9 Hz), 4.21 (1H, t, J=6.9 Hz), 2.37-2.50 (2H, m), 2.20-2.31 (1H, m), 2.04 (3H, s), 1.94-2.04 (1H, m).

¹³C NMR (100 MHz, CDCl₃), δ=171.7, 155.7, 143.7, 143.5, 141.1, 135.0, 128.5, 128.2, 127.5, 126.3, 124.9, 119.8, 67.2, 66.8, 53.1, 46.9, 31.6, 29.5, 15.2.

HRMS (ESI+) for C₂₇H₂₇NNaO₄S (+) [M+Na]+: calcd 484.1558; found 484.1552.

### Compound 1.2

NaIO₄ (8.6 g, 40.3 mmol, 1.0 equiv) in H₂O (20 mL) was slowly added to the mixture of 1.1 (18.6 g, 40.3 mmol, 1.0 equiv.) in MeOH (200 mL) and THF (40 mL) in ice bath. The cooling bath was then removed and the mixture was stirred overnight. After full conversion was achieved, the reaction mixture was concentrated under vacuum. The residue was diluted with DCM (500 mL) and washed with 1 N HCl (2×100 mL) and brine (100 mL). The organic phase was dried over anhydrous Na₂SO₄ and concentrated under vacuum. Purification by silica gel chromatography (CH₂Cl₂/MeOH=18:1) gave 1.2 (17.3 g, 90%) as a white solid.

¹H NMR (400 MHz, CDCl₃), δ=7.75 (2H, d, J=7.5 Hz), 7.59 (2H, s), 7.30-7.41 (9H, m), 5.91-5.99 (1H, m), 5.19 (2H, dd, J=12.3 Hz, 18.5 Hz), 4.50-4.52 (1H, m), 4.40 (2H, dd, J=6.6 Hz, 11.4 Hz), 4.19 (1H, t, J=6.5 Hz), 2.64-2.70 (2H, m), 2.48 (3H, d, J=6.3 Hz), 2.37-2.42 (1H, m), 2.15-2.19 (1H, m).

¹³C NMR (100 MHz, CDCl₃), δ=171.0, 156.0, 143.7, 143.5, 141.1, 134.8, 128.6, 128.3, 127.6, 127.0, 119.8, 67.5, 66.9, 53.0, 52.8, 50.0, 49.9, 47.0, 38.4, 25.9, 25.5.

HRMS (ESI+) for C₂₇H₂₇NNaO₅S(+) [M+Na]+: calcd 500.1508; found 500.1517.

### Compound 1.3

The compound 1.2 (17.3 g, 36.2 mmol) was refluxed in xylene (250 mL) for 24 h. After full conversion, the solvent was removed under vacuum and the residue was purified by silica gel chromatography (n-hexane/EtOAc=7:1) to afford 1.3 as a light yellow solid (10.0 g, 67%).

¹H NMR (400 MHz, CDCl₃), δ=7.78 (2H, d, J=7.5 Hz), 7.61 (2H, d, J=7.5 Hz), 7.28-7.42 (9H, m), 5.85-6.06 (1H, m), 5.51 (1H, d, J=7.3 Hz), 5.29-5.38 (2H, m), 5.23 (2H, s), 5.20 (1H, m), 5.44 (2H, d, J=6.8 Hz), 4.25 (1H, t, J=6.8 Hz).

¹³C NMR (100 MHz, CDCl₃), δ=170.2, 155.5, 143.7, 143.6, 141.2, 135.0, 132.1, 128.5, 128.4, 128.1, 127.6, 127.0, 125.0, 119.9, 117.8, 67.4, 67.0, 56.1, 47.0.

### Compound 2.1

DMAP (418.3 mg, 3.42 mmol, 0.1 equiv.) was added to the solution of Boc-Gly-OH 2 (6.0 g, 34.20 mmol, 1.0 equiv.), EtSH (5 mL, 68.50 mmol, 2.0 equiv.) and DCC (7.0 g, 34.2 mmol, 1.0 equiv.) in anhydrous CH₂Cl₂ under argon. The reaction mixture was stirred at room temperature for 4 h. After full conversion, the white suspension was filtrated through celite and the filtrate was concentrated under vacuum. The residue was purified by silica gel chromatography (n-hexane/EtOAc=5:1) to give 2.1 as a colorless oil (6.7 g, 90%).

¹H NMR (400 MHz, CDCl₃), δ 5.11 (s, 1H), 4.06 (d, J=6.0 Hz, 2H), 2.93 (q, J=7.4 Hz, 2H), 1.48 (s, 9H), 1.28 (t, J=7.4 Hz, 3H).

¹³C NMR (100 MHz, CDCl₃), δ 198.5, 155.4, 80.3, 50.2, 28.5, 23.2, 14.9.

HRMS (FAB) for C₇H₁₄NO₃: calcd 160.0895; found 160.0889.

### Compound 2.2

Et₃SiH (7.20 mL, 45.6 mmol, 2.0 equiv) was slowly added to the mixture of compound 2.1 (5.0 g, 22.80 mmol, 1.0 equiv.) and 10% Pd/C (500.0 mg) in anhydrous DCM under argon. The reaction mixture was stirred at room temperature for 3 h, then was filtered through celite. The filtrate was concentrated under vacuum and the residue was purified by flash silica gel chromatography (n-hexane/EtOAc=3:1) to give 2.2 as a colorless oil (3.2 g, 86%).

¹H NMR (400 MHz, CDCl₃), δ 9.58 (s, 1H), 5.41 (s, 1H), 3.99 (d, J=5.2 Hz, 2H), 1.40 (s, 9H).

¹³C NMR (100 MHz, CDCl₃), δ 197.7, 155.8, 80.0, 51.2, 28.2.

HRMS (CI+) for C₇H₁₃NO₃: calcd 159.0895; found 159.0892.

### Compound 2.3

Under argon protection, vinylmagnesium bromide solution (1M in THF, 24.7 mL, 2.0 equiv.) was added into a 100 mL round bottom flask. The solution was cooled to −30° C., then the solution of compound 2.2 (1.8 g, 12.40 mmol, 1.0 equiv.) in anhydrous THF (20 mL) was added. The resultant solution was stirred at −30° C. for 2 h, then the saturated NH4Cl aqueous solution (20 mL) was added slowly to quench the reaction. THF was evaporated under vacuum, and the residue was diluted with EtOAc (150 mL). The organic phase was washed with water (2×30 mL) and brine (30 mL). The organic phase was dried over anhydrous Na₂SO₄. The residue was purified by silica gel chromatography (n-hexane/EtOAc=3:1) to give 2.3 as a colorless oil (1.1 g, 55%).

¹H NMR (400 MHz, CDCl₃): δ=5.83 (1H, ddd, J=5.7 Hz, 6.5 Hz, 12.2 Hz), 5.33 (1H, d, J=14.6 Hz), 5.18 (1H, d, J=11.7 Hz), 5.16 (1H, m), 4.22 (1H, m), 3.28-3.37 (2H, m), 3.06-3.08 (1H. m), 1.44 (9H, s).

¹³C NMR (100 MHz, CDCl₃): δ=156.7, 138.0, 116.1, 79.6, 72.2, 46.2, 28.4.

HRMS (CI+) for C₉H₁₈O₃N: calcd 188.1286; found 188.1282.

### Compound 2.4

DMAP (44.6 mg, 0.36 mmol, 0.1 equiv.) was added to the solution of 2.3 (0.7 g, 3.65 mmol, 1.0 equiv.), Boc-Ala-OH (1.05 g, 5.45 mmol, 1.5 equiv.) and EDCI (1.4 g, 7.25 mmol, 2.0 equiv.) in anydrous DCM (15 mL) under argon. The reaction mixture was stirred at room temperature for 8 h, then was diluted with ethyl acetate (200 mL) and washed sequentially with 1 N HCl (2×30 mL) and brine (30 mL). The organic phase was dried over anhydrous Na₂SO₄ and concentrated under vacuum. The residue was purified by silica gel chromatography (n-hexane/EtOAc=7:1) to give the compound 2.4-1 (lower polarity, 0.51 g, 40%) and compound 2.4-2 (higher polarity, 0.57 g, 45%).

Compound 2.4-1: ¹H NMR (400 MHz, CDCl₃): δ=5.77 (1H, ddd, J=6.1 Hz, 10.6 Hz, 17.0 Hz), 5.29-5.35 (3H, m), 5.02 (1H, d, J=5.6 Hz), 4.91 (1H, s), 4.28 (1H, t, J=6.1 Hz), 3.45 (1H, m), 3.23-3.27 (1H, m), 1.44 (9H, s), 1.43 (9H, s), 1.40 (3H, d, J=11.3 Hz).

¹³C NMR (100 MHz, CDCl₃): δ=172.4, 156.0, 155.4, 133.1, 118.6, 80.1, 79.6, 74.6, 49.7, 43.6, 28.4, 18.4.

HRMS (ESI+) for C₁₇H₃₀N₂NaO₆(+) [M+Na]+: calcd 381.1996; found 381.2002.

Compound 2.4-2: ¹H NMR (400 MHz, CDCl3): δ=5.77 (1H, ddd, J=5.8 Hz, 10.6 Hz, 16.9 Hz), 5.24-5.37 (3H, m), 5.00-5.03 (2H, m), 4.26 (1H, t, J=6.6 Hz), 3.20-3.27 (1H, m), 1.44 (9H, s), 1.42 (9H, s), 1.38 (3H, d, J=7.2 Hz).

¹³C NMR (100 MHz, CDCl3): δ 172.7, 156.0, 155.3, 133.2, 118.5, 80.2, 79.7, 74.8, 49.6, 43.8. 28.5, 18.3.

HRMS (ESI+) for C₁₇H₃₀N₂NaO₆(+) [M+Na]+: calcd 381.1996; found 381.2002.

### Compound 2.3-1

1 M aqueous lithium hydroxide (36.7 mg, 0.90 mmol, 3.0 equiv.) was added to the solution of compound 2.4-1 (100.0 mg, 0.30 mmol, 1.0 equiv.) in THF at room temperature. The mixture was stirred for 4 h and extracted with ethyl acetate (3×30 mL). The combined organic phase was washed with aqueous sodium bicarbonate (30 mL) and brine (20 mL), and dried over anhydrous Na₂SO₄. The organic phase was concentrated under vacuum, and the residue was purified by silica gel chromatography (n-hexane/EtOAc=2.5:1) to give the 2.3-1 as a colorless oil (40.3 mg, 92%).

¹H NMR (400 MHz, CDCl₃), δ=5.83 (1H, ddd, J=5.7 Hz, 6.5 Hz, 12.2 Hz), 5.33 (1H, d, J=14.6 Hz), 5.18 (1H, d, J=11.7 Hz), 5.16 (1H, m), 4.22 (1H, m), 3.28-3.37 (2H, m), 3.06-3.08 (1H. m), 1.44 (9H, s).

¹³C NMR (100 MHz, CDCl₃): δ=156.8, 138.0, 116.1, 79.6, 72.2, 46.2, 28.4.

[α]D25=−2.69o (c=1.40, CHCl₃)

HRMS (CI+) for C₉H₁₈O₃N: calcd 188.1286; found 188.1282.

### Compound 2.3-2

The 1 M aqueous lithium hydroxide (36.7 mg, 0.90 mmol, 3.0 equiv.) solution was added to the solution of compound 2.4-2 (100 mg, 0.30 mmol, 1.0 equiv.) in THF at room temperature. The mixture was stirred overnight and extracted with ethyl acetate (3×30 mL). The combined organic phase was washed with aqueous sodium bicarbonate (30 mL) and brine (20 mL), and dried over anhydrous Na₂SO₄. The organic phase was concentrated under vacuum and purified by silica gel chromatography (n-hexane/EtOAc=2.5:1) to give 2.3-2 as a colorless oil (40.7 mg, 81%).

¹H NMR (400 MHz, CDCl₃): δ=5.83 (1H, ddd, J=5.7 Hz, 6.5 Hz, 12.2 Hz), 5.33 (1H, d, J=14.6 Hz), 5.18 (1H, d, J=11.7 Hz), 5.16 (1H, m), 4.22 (1H, m), 3.28-3.37 (2H, m), 3.06-3.08 (1H. m), 1.44 (9H, s).

¹³C NMR (100 MHz, CDCl₃), δ=156.8, 138.0, 116.1, 79.6, 72.2, 46.2, 28.4.

HRMS (CI+) for C₉H₁₈O₃N: calcd 188.1286; found 188.1281.

### Compound 2.3-2a

To a stirred solution of compound 2.3-2 (100 mg, 0.50 mmol, 1.0 equiv.), triphenylphosphine (168 mg, 0.60 mmol, 1.2 equiv.) and p-nitrobenzoic acid (107 mg, 0.60 mmol, 1.2 equiv.) in anhydrous THF at 0° C., was added diisopropyl azodicarboxylate (126 μL, 0.60 mmol, 1.2 equiv.). The reaction mixture was gradually warmed to room temperature and allowed to be stirred for 1 h. The solution was then concentrated under vacuum, and the residue was purified by silica gel chromatography (n-hexane/EtOAc=5:1) to give 2.3-2a as a yellow solid (134.8 mg, 75%).

¹H NMR (400 MHz, CDCl₃): δ=8.30 (2H, dd, J=1.9 Hz, 6.9 Hz), 8.23 (2H, dd, J=2.1 Hz, 6.9 Hz), 5.88 (1H, ddd, J=6.1 Hz, 10.5 Hz, 17.0 Hz), 5.57 (1H, dd, J=6.0 Hz, 11.3 Hz), 5.30-5.45 (2H, m), 4.74 (1H, s), 3.48-3.55 (2H, m), 1.44 (9H, s).

¹³C NMR (100 MHz, CDCl₃): δ=164.0, 155.9, 150.8, 135.6, 133.1, 131.0, 123.7, 119.2, 79.9, 75.6, 43.6, 28.4.

HRMS (ESI+) for C₁₆H₂₀N₂NaO₆(+) [M+Na]+: calcd 359.1214; found 359.1213.

### Compound 2.3-1

The 1 M aqueous lithium hydroxide (63 mg, 1.50 mmol, 3.6 equiv.) was added to the solution of compound 2.3-2a (135 mg, 0.42 mmol, 1.0 equiv.) in THF at room temperature. The mixture was stirred for 4 h and extracted with ethyl acetate (3×30 mL). The combined organic phase was washed with aqueous sodium bicarbonate (30 mL) and brine (20 mL), and dried over anhydrous Na₂SO₄. The organic phase was concentrated under vacuum and purified by silica gel chromatography (n-hexane/EtOAc=2.5:1) to give 2.3-1 as a colorless oil (50.1 mg, 90%).

### Compound 3

Grubbs II catalyst (88 mg, 0.10 mmol, 0.019 equiv.) and CuI (102 mg, 0.53 mmol, 0.1 equiv.) were added to a flame-dried flask equipped with a condenser, and anhydrous diethyl ether (10 mL) was added. Compound 2.4-1 (1.00 g, 5.33 mmol, 1.0 equiv.) and compound 1.3 (4.40 g, 10.74 mmol, 2.0 equiv.) were dissolved in anhydrous diethyl ether (15 mL), and this solution was added via syringe to the above flask. The mixture was refluxed for 12 h. After full conversion of 2.4-1, the mixture was concentrated under vacuum and purified by silica gel chromatography (n-hexane/EtOAc=2:1) to give 3 as a brown solid (1.80 g, 60%).

¹H NMR (400 MHz, CDCl₃): δ=7.76 (2H, d, J=7.5 Hz), 7.59 (2H, d, J=7.2 Hz), 7.25-7.42 (9H, m), 5.84 (1H, dd, J=4.7 Hz, 8.7 Hz), 5.76 (1H, dd, J=5.1 Hz, 8.7 Hz), 5.54 (1H, d, J=7.5 Hz), 5.19 (2H, s), 4.97 (1H, m), 4.85 (1H, s), 4.39 (2H, dd, J=2.32 Hz, 10.0 Hz), 4.19-4.23 (2H, m), 3.15-3.25 (1H, m), 2.95-3.05 (1H, m), 1.43 (9H, s).

¹³C NMR (100 MHz, CDCl3), δ=170.7, 157.1, 156.0, 144.0, 141.5, 136.2, 134.0, 128.9, 128.7, 128.5, 128.0, 127.4, 127.0, 126.3, 120.2, 80.0, 71.3, 67.8, 67.4, 55.6, 47.3, 46.4, 28.6.

HRMS (ESI+) for C₃₃H₃₆N₂O₇Na(+) [M+Na]+: calcd 595.2415; found 595.2408.

### Compound 4

Compound 3 (1.80 g, 3.15 mmol, 1.0 equiv.) and catalytic amount of Pd/C (208 mg) were mixed with ethyl acetate (20 mL). The mixture was stirred under H₂ atmosphere at r.t. for 5 h. After full conversion of 3, the reaction mixture was filtered through celite, and the filtrate was concentrated under vacuum, to give the yellow solid. The crude product compound 3.1 was used in the next step without further purification.

The crude compound 3.1 was dissolved in anhydrous DMF (10 mL) at room temperature, then the KHCO₃ (710 mg, 7.10 mmol, 2.3 equiv.) and benzyl bromide (621 μL, 5.31 mmol, 1.7 equiv.) were added. The mixture was stirred at room temperature for 4 h. The solution was diluted with 1 N HCl (50 mL) and extracted with EtOAc (3×100 mL). The combined organic phase was washed with brine (50 mL) and dried over anhydrous Na₂SO₄. The solution was concentrated under vacuum, and the residue was purified by silica gel chromatography (n-hexanes/EtOAc=2:1) to give 4 as a white solid (1.40 g, 75%).

¹H NMR (400 MHz, CDCl₃): δ=7.76 (2H, d, J=7.5 Hz), 7.63 (2H, d, J=7.5 Hz), 7.25-7.41 (9H, m), 5.63 (1H, d, J=5.3 Hz), 5.14-5.22 (2H, m), 4.91 (1H, s), 4.33-4.45 (3H, m), 4.20 (1H, t, J=6.8 Hz), 3.66 (1H, s), 3.19 (1H, m), 2.96 (1H, t, J=6.2 Hz), 2.02-2.06 (1H, m), 1.75-1.80 (1H, m), 1.43 (2H, m), 1.43 (9H, d).

¹³C NMR (100 MHz, CDCl₃): δ=172.3, 157.1, 156.3, 144.0, 143.8, 141.4, 135.3, 128.8, 128.7, 127.8, 127.2, 125.2, 120.1, 79.9, 71.2, 67.4, 67.2, 53.8, 47.3, 46.7, 30.3, 29.2, 28.5.

HRMS (ESI+) for C₃₃H₃₈N₂NaO₇(+) [M+Na]⁺: calcd 597.2571; found 597.2571.

### Results

Human adiponectin is a polypeptide with 244 amino acids and contains four structurally distinct domains from NH₂- to COOH-terminus: a signal peptide, a variable region, a collagenous domain, and a globular domain (FIG. 1). The collagenous domain is glycosylated with 2-O-α-D-glucopyranosyl-D-galactose disaccharide via hydroxylysine¹²⁻¹³. Specifically, the mammalian adiponectin collagenous domain contains four 5-(2S,5R)-hydroxylysine residues (at positions 65, 68, 77, 101) which are glycosylated with the glucosyl-galactose disaccharide (FIG. 1). The glycan structure is very different from the common O-linked or N-linked glycoproteins which represents a huge challenge to covert the full-length human adiponectin protein into a viable drug via recombinant approaches. Although efforts have been made to identify a minimal structure capable of eliciting the required pharmacological agonist activities, the structure-activity relationships of the individual domain within adiponectin have not been well defined¹⁴⁻¹⁹.

Prior to undertaking chemical synthesis of adiponectin, a first task was to undertake a large-scale synthesis of the (2S, 5R)-hydroxylysine building block. Although several synthetic routes have been previously reported, it is likely that none of them would be applicable to large-scale synthesis²⁶⁻²⁸. The synthesis employed in this study is shown in Scheme 1.

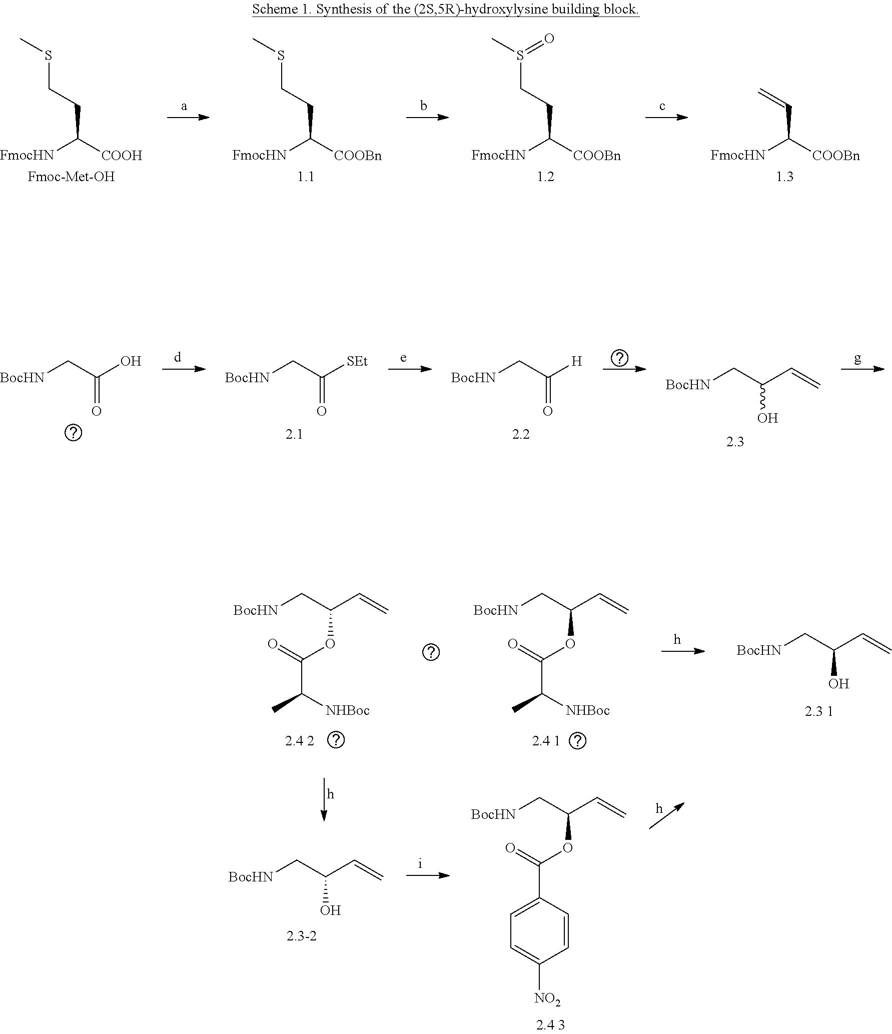

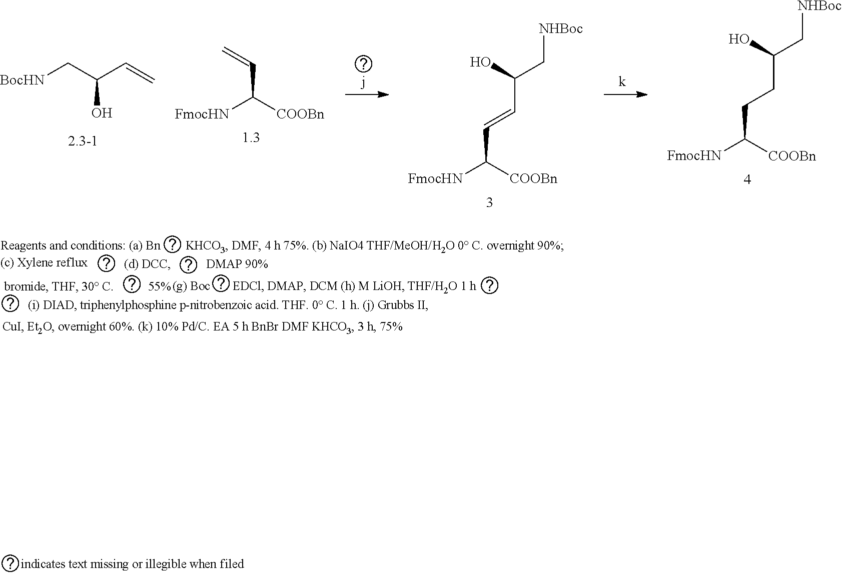

An important step of this synthetic strategy was the reaction between L-α-vinylglycine (1.3) and 1-amino-3-butene-2-ol derivative (2.3-1) via olefin metathesis. The carboxylic acid of Fmoc-Met-OH was first masked by a Bn group to give 1.1, which was converted to 1.2 by NaIO₄ oxidation. This intermediate underwent pyrolysis to afford the corresponding product 1.3 in overall 50% yield. Boc-Gly thioester (2.1) was subjected to the Fukuyama reduction²⁹, and the resultant aldehyde (2.2) underwent a vinyl magnesium addition to form a racemic alcohol (2.3). Subsequently, diastereomeric resolution was used via forming diastereomeric esters 2.4-1 and 2.4-2, which could be readily separated and purified by silica gel column. The ester compound 2.4-1 was treated with LiOH to regenerate the optical pure product 2.3-1, allowing cross olefin metathesis³⁰ via the Grubbs' catalyst with compound 1.3 to produce the precursor 3, which underwent hydrogenation and reinstallation of Bn group to obtain (2S,5R)-hydroxylysine building block 4 (HLBB) with orthogonal protecting groups. Moreover, the undesired diastereomer 2.4-2 with inverse chiral center could be transformed into the desired one. This synthetic route can be efficiently performed up to 10 grams of the product.

### Example 2: Synthesis of Glycosylated Hydroxylysine

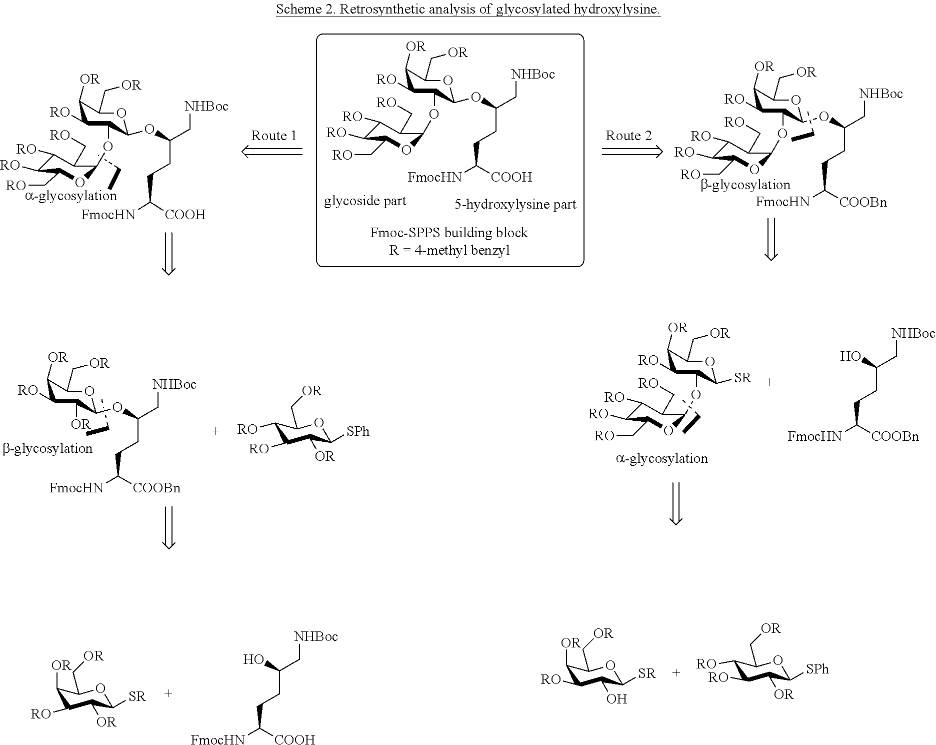

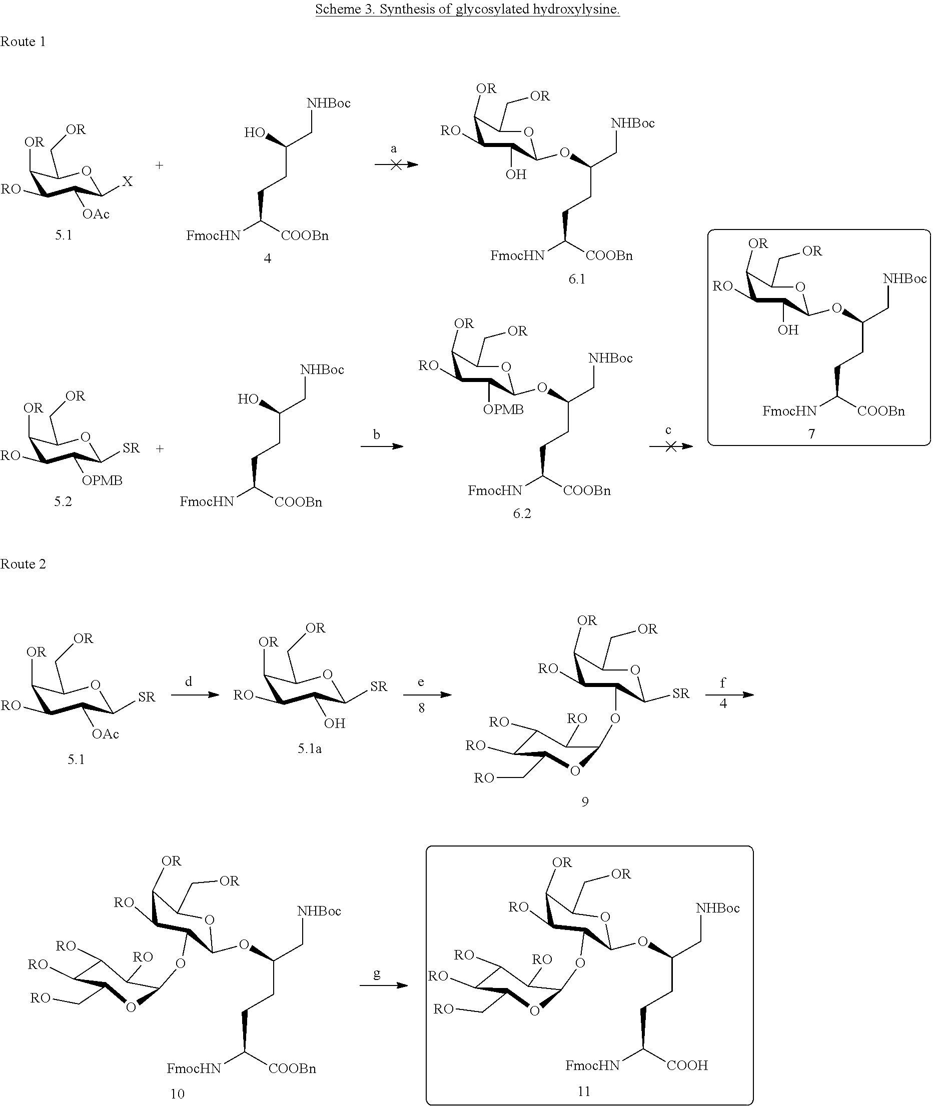

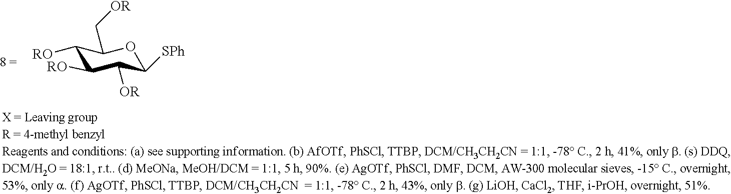

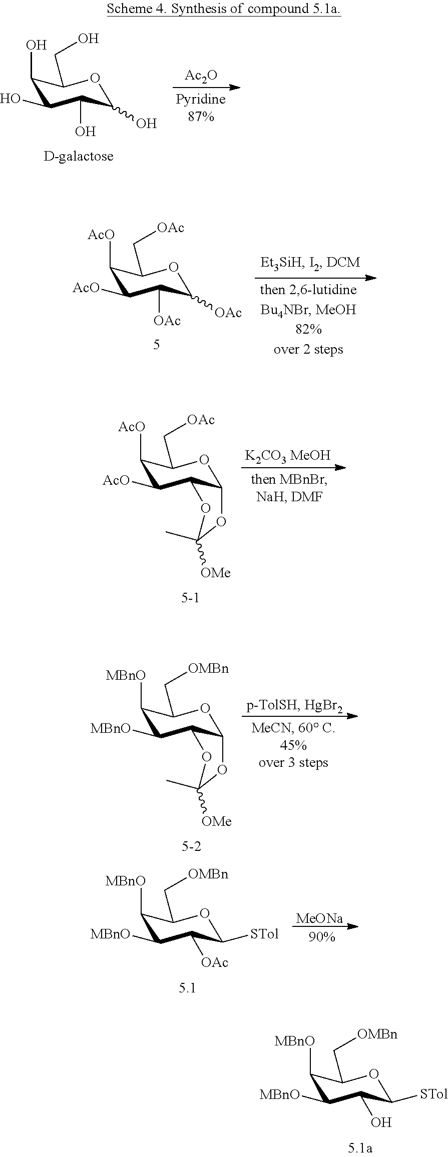

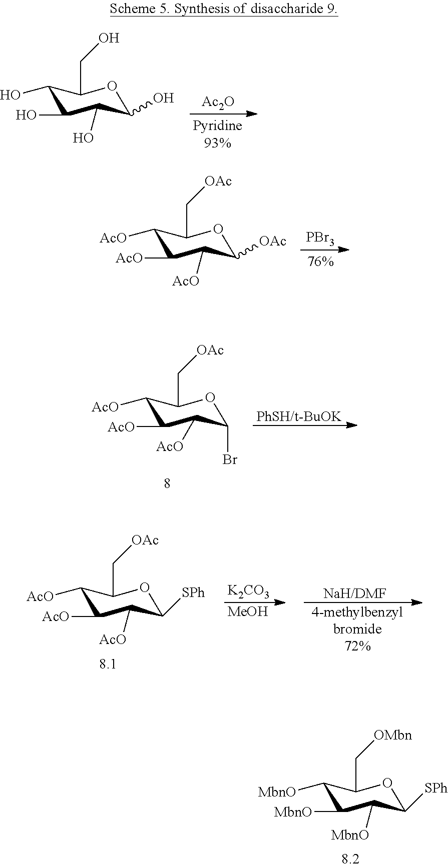

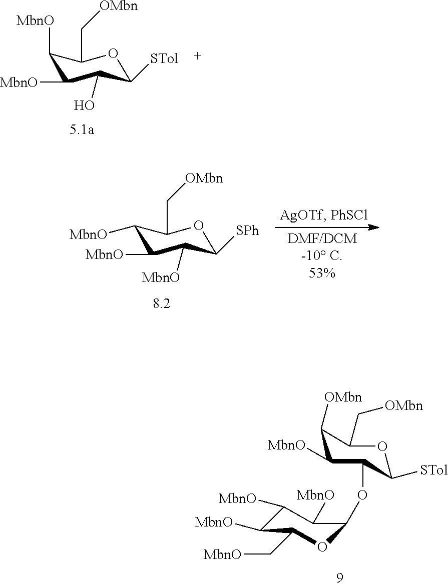

### Material and Methods

All glycosylation reactions were conducted under argon using flame-dried molecular sieves.

### Compound 5.1

Acetic anhydride (15.0 mL, 112.5 mmol, 7.5 equiv.) was added to a stirred slurry of D-galactose 3.4 (2.70 g, 15.0 mmol, 1.0 equiv.) in anhydrous pyridine (10 mL) at room temperature, followed by catalytic amount of DMAP (183 mg, 1.47 mmol, 0.1 equiv.). The mixture was stirred at room temperature for 4 h, then the solvent was evaporated under vacuum. The residue was diluted with EtOAc (250 mL), washed sequentially with 1N HCl (3×40 mL), saturated NaHCO₃ (2×40 mL), and brine (2×40 mL). The organic phase was dried over Na₂SO₄ and concentrated under vacuum to give crude compound 5 as a colorless oil (5.10 g, 87%), which was directly used without further purification.

i) The crude compound 5 (1.00 g, 2.51 mmol, 1.0 equiv.) was dissolved in anhydrous CH₂Cl₂ (8 mL). To the above solution were added I₂ (912 mg, 3.62 mmol, 1.5 equiv.) and triethylsilane (571 μL, 3.60 mmol, 1.5 equiv.). The mixture was refluxed until complete consumption of peracetylated sugar as indicated by TLC. The solution was then cooled to room temperature and subjected to the next step without workup.

ii) To the above reaction mixture were sequentially added 2,6-lutidine (1.20 mL, 10.2 mmol, 4.0 equiv.), anhydrous methanol (600 μL, 15.4 mmol, 6.1 equiv.) and tetrabutylammonium bromide (330 mg, 1.0 mmol, 0.4 equiv.). The mixture was stirred overnight at room temperature. When full conversion was achieved, the mixture was concentrated under vacuum, and the residue was purified by silica gel chromatography (n-hexane/EtOAc=5:1) to give compound 5-1 as a colorless oil (758 mg, 82% over 2 steps).

iii) The compound 5-1 (758 mg, 2.11 mmol, 1.0 equiv.) was dissolved in MeOH (4 mL), then K₂CO₃ (58 mg, 0.42 mmol, 0.2 equiv.) was added. The above mixture was stirred at room temperature for 2 h. After completion of the reaction, the solvent was evaporated under vacuum and the resulting residue was re-dissolved in the dry DMF (4 mL). To the above solution was slowly added NaH (418.6 mg, 10.55 mmol, 5.0 equiv.) at 0° C., and the resulting mixture was stirred for 15 min, followed by addition of BnBr (1.20 mL, 10.53 mmol, 5.0 equiv.) slowly. The mixture was stirred at room temperature for 6 h until full conversion. The reaction was quenched by saturated NH₄Cl aqueous solution (30 mL), and then extracted by EtOAc (3×40 mL). The combined organic phase was washed with brine (30 mL) and dried over anhydrous Na₂SO₄. The solvent was concentrated under vacuum and the resulting crude product compound 5-2 was subjected to the next step without further purification.

iv) The crude compound 5-2 was dissolved in anhydrous acetonitrile (8 mL), then p-toluenethiol (1.30 g, 10.55 mmol, 1.0 equiv.) and HgBr₂ (76 mg, 0.21 mmol, 0.02 equiv.) were added. The reaction mixture was stirred at 60° C. for 5 h, then was concentrated under vacuum. The residue was purified by silica gel chromatography (n-hexane/EtOAc=5:1) to give compound 5.1 as a white solid (602 mg, 45% over 2 steps).

¹H NMR (400 MHz, CDCl₃): δ=7.41 (2H, d, J=8.1 Hz), 7.13-7.22 (12H, m), 7.05 (2H, d, J=8.0 Hz), 5.41 (1H, t, J=9.8 Hz), 4.91 (1H, d, J=11.4 Hz), 4.65 (1H, d, J=12 Hz), 4.50-4.59 (3H, m), 4.41 (2H, q, J=11.4 Hz), 3.97 (2H, d, J=2.5 Hz), 3.58-3.66 (3H, m), 3.54 (1H, dd, J=2.7 Hz, 9.6 Hz), 2.37-2.39 (9H, m), 2.33 (3H, s), 2.09 (3H, s).

¹³C NMR (100 MHz, CDCl₃): δ=169.52, 137.54, 137.49, 137.09, 135.52, 134.91, 134.88, 132.35, 129.97, 129.53, 129.12, 129.10, 128.85, 128.22, 128.10, 127.63, 87.13, 81.25, 77.68, 77.29, 74.13, 73.47, 72.40, 71.74, 69.87, 68.73, 21.22, 21.21, 21.15, 21.13.

HRMS (ESI+) for C₃₉H₄₄NaO₆S (+) [M+Na]⁺: calcd 663.2756; found 663.2749.

### Compound 5.1a

The compound 5.1 (602 mg, 0.94 mmol, 1.0 equiv.) was dissolved in MeOH (10 mL), then 25% NaOMe in MeOH (90 μL, 0.30 mmol, 0.3 equiv.) was added. The reaction mixture was stirred at room temperature for 6 h, and was quenched by HOAc (30 μL). The resulting mixture was diluted with ethyl acetate (150 mL) and washed with H₂O (20 mL) and brine (20 mL). The organic phase was dried over anhydrous Na₂SO₄ and concentrated under vacuum. The residue was purified by silica gel chromatography (n-hexane/EtOAc=4:1) to give compound 5.1 as a white solid (507 mg, 90%).

¹H NMR (400 MHz, CDCl₃): δ=7.48 (2H, d, J=8.0 Hz), 7.27 (2H, d, J=7.9 Hz), 7.14-7.23 (10H, m), 7.05 (2H, d, J=8.0 Hz), 4.87 (1H, d, J=11.4 Hz), 4.73 (1H, d, J=11.7 Hz), 4.62 (1H, d, J=11.7 Hz), 4.56 (1H, d, J=11.3 Hz), 4.42-4.52 (3H, m), 3.98 (2H, q, J=9.6 Hz), 3.63-3.68 (3H, m), 3.46 (1H, dd, J=2.5 Hz, 9.2 Hz), 2.39 (6H, s), 2.38 (3H, s), 2.34 (3H, s).

¹³C NMR (100 MHz, CDCl₃): δ=137.69, 137.62, 137.56, 137.05, 135.70, 134.99, 134.88, 132.77, 129.61, 129.25, 129.14, 128.84, 128.13, 127.99, 127.87, 88.77, 83.08, 77.65, 74.19, 73.47, 72.76, 72.14, 69.00, 68.61, 21.23, 21.16.

HRMS (ESI+) for C₃₇H₄₂NaO₅S (+) [M+Na]⁺: calcd 621.2651; found 621.2641.

### Compound 8.2

i) Acetic anhydride (39.0 mL, 417 mmol, 7.5 equiv.) was added to a stirred slurry of D-glucose 3.26 (10.0 g, 55.6 mmol, 1.0 equiv.) in anhydrous pyridine (34.0 mL, 417 mmol, 7.5 equiv.) at room temperature, followed by catalytic amount of DMAP (677 mg, 5.6 mmol, 0.1 equiv.). The mixture was stirred at room temperature for 12 h, then ethanol (10 mL) was added and stirred for another 1 h to quench the excess amount of acetic anhydride. The solvent was evaporated under vacuum, and the residue was diluted with EtOAc (500 mL). The organic phase was washed with 1N HCl (3×80 mL), saturated NaHCO₃ (2×80 mL), and brine (2×80 mL). The organic phase was then dried over Na₂SO₄ and concentrated under vacuum to give glucose petaacetate as a white solid (20.1 g, 93%).

ii) The glucose pentaacetate (20.1 g, 51.3 mmol, 1.0 equiv.) was dissolved in HOAc (50 mL) under ice bath, then PBr₃ (7.6 mL, 78.8 mmol, 1.5 equiv.) was slowly added. To this mixture, H₂O (4.4 mL, 239 mmol, 4.6 equiv.) was added slowly in 30 min. The reaction mixture was stirred at room temperature for 3 h. After full conversion of material, the mixture was diluted with n-hexane/CH₂Cl₂=2:1 (800 mL in total), and sequentially washed with ice water (500 mL), saturated NaHCO₃ (2×300 mL), and brine (500 mL). The organic phase was dried over Na₂SO₄ and concentrated under vacuum to give glucosyl bromide 8 intermediate as a colorless oil (16.0 g, 76%). The crude product was used in the next step without further purification.

iii) Potassium tert-butoxide (4.37 g, 38.9 mmol, 1.0 equiv.) was added to the solution of PhSH (4.0 mL, 38.9 mmol, 1.0 equiv.) in anhydrous THF (50 mL) at 0° C. The reaction mixture was stirred at room temperature for 0.5 h, then compound 8 (16.0 g, 38.9 mmol, 1.0 equiv.) in THF (50 mL) was slowly added. After full conversion of 8, the reaction mixture was diluted with EtOAc (800 mL) and washed with H₂O (3×400 mL) and brine (2×400 mL). The organic phase was dried over Na₂SO₄ and concentrated under vacuum. The residue was purified by silica gel chromatography (n-hexane/EtOAc=2.5:1) to give compound 8.1 as a white solid (15.2 g, 90%).

iv) K₂CO₃ (952 mg, 6.9 mmol, 0.2 equiv.) was added to the solution of compound 8.1 (15.2 g, 34.5 mmol, 1.0 equiv.) in MeOH (100 mL). The reaction was stirred at room temperature for 2 h. After full conversion of the material, the solvent was evaporated under vacuum, and the residue was re-dissolved in anhydrous DMF (80 mL). The NaH (60% dispersed in mineral oil) (6.90 g, 172.5 mmol, 5.0 equiv.) was added slowly to this solution, and the resulting slurry was stirred for 30 min at 0° C. To this slurry, the 4-methylbenzyl bromide (32.0 g, 172.5 mmol, 5.0 equiv.) was slowly added, and the mixture was stirred at room temperature overnight. The mixture was diluted with EtOAc (1000 mL) and washed with 1N HCl (3×400 mL) and brine (2×400 mL). The organic phase was dried over anhydrous Na₂SO₄ and concentrated under vacuum. The residue was purified by silica gel chromatography (n-hexane/EtOAc=6:1) to give compound 8.2 as a white solid (17.1 g, 72%).

¹H NMR (400 MHz, CDCl₃): δ=7.59-7.62 (2H, m), 7.31 (2H, d, J=7.9 Hz), 7.22-7.27 (7H, m), 7.14-7.18 (6H, m), 7.10 (4H, q, J=8.3 Hz), 4.85 (3H, m), 4.70-4.80 (2H, m), 4.66 (1H, d, J=9.8 Hz), 4.50-4.61 (3H, m), 3.67-3.79 (3H, m), 3.62 (1H, t, J=9.4), 2.38 (3H, s), 2.37 (6H, s), 2.35 (3H, s).

¹³C NMR (100 MHz, CDCl₃): δ=137.59, 137.53, 137.40, 137.22, 135.47, 135.24, 135.07, 133.97, 131.91, 129.14, 129.11, 129.09, 129.02, 128.88, 128.38, 128.13, 128.13, 127.98, 127.84, 127.36, 87.50, 86.64, 80.69, 79.10, 77.66, 77.24, 75.72, 75.28, 74.92, 73.28, 68.82, 21.23, 21.21.

HRMS (ESI+) for C₄₄H₄₈NaO₅S (+) [M+Na]⁺: calcd 711.3120; found 711.3112.

### Compound 9

Compound 8.2 (1.03 g, 1.50 mmol, 1.5 equiv.) and flame-dried molecular sieve (AW-300) was suspended in anhydrous CH₂Cl₂ (25 mL). Then, DMF (462 μL, 6.00 mmol, 6.0 equiv.) was added to the mixture. The resulting mixture was stirred at room temperature for 10 min and at −10° C. for an additional 10 min. Subsequently, AgOTf (385 mg, 1.50 mmol, 1.5 equiv.) was added, and then phenylsulfenylchloride (217 mg, 1.50 mmol, 1.5 equiv.) was slowly added. Upon completion of the activation of glycosyl donor 8.2, thioglycoside acceptor 5.1a (599 mg, 1.00 mmol, 1.0 equiv.) was added to the reaction mixture, and the mixture was stirred at −10° C. overnight. The mixture was then diluted with EtOAc (300 mL) and washed by 1N HCl (3×50 mL) and brine (100 mL). The organic phase was dried over anhydrous Na₂SO₄ and concentrated under vacuum. The residue was purified by silica gel chromatography (n-hexane/EtOAc=6:1) to give compound 9 as a colorless oil (624 mg, 53%).

¹H NMR (400 MHz, CDCl₃): δ=7.41 (2H, d, J=8.2 Hz), 7.24-7.28 (4H, m), 7.15-7.20 (12H, m), 7.06-7.14 (8H, m), 6.98 (4H, t, J=8.6 Hz), 6.88 (2H, d, J=7.8 Hz), 5.78 (1H, d, J=3.8 Hz), 4.88-4.97 (3H, m), 4.80 (3H, t, J=9.6 Hz), 4.66-4.75 (3H, m), 4.49-4.57 (2H, m), 4.39-4.48 (3H, m), 4.30-4.34 (3H, m), 4.21 (1H, d, J=12 Hz), 4.06 (1H, d, J=2.4 Hz), 3.95 (3H, t, J=9.3 Hz), 3.60-3.66 (5H, m), 3.24 (2H, s), 2.40 (3H, s), 2.39 (9H, s), 2.36 (3H, s), 2.34 (3H, s), 2.31 (3H, s), 2.27 (3H, s).

¹³C NMR (100 MHz, CDCl₃): δ=137.57, 137.26, 137.11, 137.07, 136.98, 136.83, 136.81, 136.09, 135.90, 135.20, 135.03, 134.88, 134.15, 131.16, 130.10, 129.64, 129.14, 129.12, 129.07, 129.03, 128.99, 128.98, 128.93, 128.84, 128.76, 128.70, 128.51, 128.20, 128.17, 128.10, 127.90, 127.87, 96.18, 87.57, 82.75, 81.95, 79.59, 75.48, 74.66, 74.30, 73.52, 73.40, 73.18, 72.99, 72.48, 72.14, 71.70, 69.99, 68.66, 67.70, 21.25, 21.23, 21.18, 21.15, 21.10.

HRMS (ESI+) for C₇₅H₈₄NaO₁₀S (+) [M+Na:]⁺ calcd 1199.5683; found 1199.5687.

### Compound 10

The suspension of compound 9 (624 mg, 0.53 mmol, 1.0 equiv.), compound 4 (609 mg, 1.06 mmol, 2.0 equiv.), AgOTf (136 mg, 0.53 mmol, 1.0 equiv.), 2,4,6-tri-tert-butylpyrimidine (131 mg, 0.53 mmol, 1.0 equiv.) and flame-dried 4 Å molecular sieves (2.00 g) in anhydrous propionitrile (18 mL) and anhydrous DCM (6 mL) was stirred for 30 min under argon. The mixture was then cooled to −78° C., and benzenesulfenyl chloride (71 μL, 1.25 mmol, 2.4 equiv.) was added slowly in 15 min. The reaction mixture was stirred for 1.5 h at −78° C. and quenched by Et₃N after full conversion of compound 9. The suspension was filtered through celite, and the filtering cake was thoroughly washed with EtOAc (150 mL). The filtrate was washed with saturated NaHCO₃ (2×30 mL) and brine (30 mL). The organic phase was dried over anhydrous Na₂SO₄ and concentrated under vacuum. The residue was purified by silica gel chromatography (n-hexane/EtOAc=3:1) to give compound 10 as a white solid (371 mg, 43%). The excess amount of compound 4 was recovered.

¹H NMR (400 MHz, CDCl₃): δ=7.78 (2H, d, J=8.0 Hz), 7.62 (m, dd, J=8.0 Hz), 7.41 (2H, t, J=8), 7.31-7.35 (7H, m), 7.06-7.23 (24H, m), 6.99 (2H, d, J=7.6 Hz), 6.84 (2H, d, J=8.6 Hz), 5.62 (1H, s), 5.54 (1H, t, J=6.3 Hz), 5.14 (2H, s), 4.88 (1H, d, J=10.6), 4.72-4.83 (4H, m), 4.49-4.62 (6H, m), 4.16-4.40 (9H, m), 4.00 (1H, t, J=9.6 Hz), 3.88 (1H, s), 3.44-3.71 (9H, m), 3.24-3.29 (2H, m), 2.33-2.39 (21H, m), 1.65-1.79 (4H, m), 1.44 (9H, s).

¹³C NMR (100 MHz, CDCl₃): δ=172.13, 156.88, 156.24, 144.05, 143.95, 141.23, 137.62, 137.33, 137.29, 137.25, 137.13, 137.05, 136.85, 135.99, 135.90, 135.50, 135.15, 135.10, 134.68, 134.36, 129.17, 129.10, 129.08, 128.97, 128.89, 128.80, 128.56, 128.55, 128.37, 128.30, 128.20, 128.16, 128.11, 128.00, 127.75, 127.63, 127.18, 127.13, 125.39, 125.31, 119.88, 102.67, 95.64, 82.07, 81.33, 79.40, 79.12, 78.80, 77.89, 75.36, 74.78, 74.28, 73.48, 73.16, 73.01, 72.87, 72.59, 72.19, 69.77, 68.98, 67.89, 67.17, 67.02, 54.03, 47.07, 42.79, 29.74, 28.74, 28.08, 27.60, 21.22, 21.20, 21.18, 21.15, 21.11.

HRMS (ESI+) for C₉₄H₁₀₈N₂O₁₇ (+) [M+H]⁺: calcd 1628.8229; found 1628.8259.

### Compound 11

To the solution of compound 10 (371 mg, 0.228 mmol, 1.0 equiv.) in isopropanol (12.5 mL) and THF (3 mL), was added powdered CaCl₂) (1.40 g, 12.5 mmol, 54.8 equiv.). To this mixture, LiOH·H₂O (140 mg, 3.3 mmol, 14.5 equiv.) dissovled in water (3.5 mL) was added. The mixture was vigorously stirred at room temperature overnight. The pH was then adjusted to 2 with 1% HCl and the organic solvents were removed under vacuum. The aqueous layer was extracted with ethyl acetate (150 mL) and the organic layer was washed with water and brine. The organic phase was dried over anhydrous Na₂SO₄ and concentrated under vacuum. The product was purified by flash chromatography (n-hexane/EtOAc=1:1 with 0.1% acetic acid) to yield compound 11 (179 mg, 51%) as a white solid, and the unreacted compound 10 was recovered.

¹H NMR (400 MHz, CDCl₃): δ=7.75 (2H, d, J=8.0 Hz), 7.60-7.61 (2H, m), 7.38-7.41 (2H, m), 7.28-7.34 (2H, m), 7.10-7.19 (24H, m), 7.00 (2H, d, J=4.0 Hz), 6.85 (2H, d, J=8.0 Hz), 5.77-5.84 (2H, m), 5.63 (1H, s), 4.71-4.96 (6H, m), 4.46-4.64 (6H, m), 4.27-4.39 (5H, m), 4.13-4.23 (3H, m), 3.99-4.03 (1H, m), 3.75-3.85 (1H, m), 3.40-3.72 (9H, m), 3.21-3.30 (1H, m), 2.25-2.39 (21H, m), 1.60-1.86 (4H, m), 1.45 (9H, s).

¹³C NMR (101 MHz, CDCl₃): δ=174.47, 156.91, 156.31, 144.02, 143.95, 141.23, 137.67, 137.33, 137.27, 137.03, 136.91, 135.92, 135.90, 135.44, 135.10, 134.92, 134.48, 134.40, 129.18, 129.10, 129.04, 128.98, 128.98, 128.91, 128.82, 128.57, 128.45, 128.28, 128.22, 128.07, 127.99, 127.88, 127.65, 127.61, 127.16, 127.15, 125.37, 125.34, 119.91, 119.86, 102.73, 95.49, 81.99, 81.32, 79.42, 79.23, 77.81, 77.29, 75.34, 74.84, 74.20, 73.39, 73.36, 73.12, 73.09, 72.56, 72.52, 72.21, 69.72, 69.01, 67.77, 67.08, 53.63, 47.16, 47.07, 43.45, 28.70, 28.51, 21.24, 21.20, 21.17, 21.16, 21.12.

HRMS (ESI+) for C₁₀₁H₁₁₅N₂O₁₆S (+) [M+H]⁺: calcd 1538.7760; found 1538.7781.

### Results

A second challenge is the scalable preparation of glycosylated hydroxylysine. According to the retrosynthetic analysis (Scheme 2), the designed glycosylated hydroxylysine building block could be synthesized via a stepwise approach (Route 1) or convergent approach (Route 2).

The protecting groups for the disaccharide need to be compatible with Fmoc-Solid Phase Peptide Synthesis (SPPS). Benzyl group (Bn) was first used, but it proved very difficult to remove from the final glycopeptides. In the end, a more acid-labile group, 4-methyl benzyl (MBn), was used.

The feasibility of the straightforward linear synthesis (Scheme 3, Route 1) was investigated. Galactosyl donor 5.1 with 2-Ac protecting groups to promote P-selectivity via neighboring group participation effect was employed to glycosylate compound 4. However, the glycosylation product 6.1 was not observed due to the formation of the corresponding ortho-ester. Several glycosyl donors and glycosylation conditions were attempted to overcome this synthetic obstacle, yet there was not any improvement to suppress the ortho-ester formation (Table 1). To overcome this obstacle, p-methoxy benzyl (PMB) was used as in compound 5.2. Without the neighboring group participation, the β-1,2-trans-O-glycosylation was achieved with assistance of the solvent effect.

TABLE 1
Glycosylation donors and glycosylation conditions tested for the synthesis of
compound 6.1.

| | Glycosylation donor | Condition | Result |
|---|---|---|---|
| 1 | 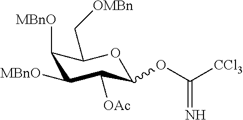 | TBSOTf, −40° C. DCM, flame-dried 4 Å molecular sieves | Orthoester (42%) |
| 2 | 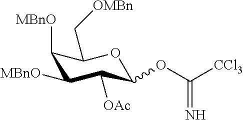 | TMSOTf, −40° C. DCM, flame-dried 4 Å molecular sieves | Orthoester (38%) |
| 3 | 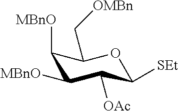 | NIS, TMSOTf, −78° C. DCM, flame-dried 4 Å molecular sieves | Orthoester (32%) |
| 4 | 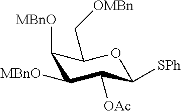 | NIS, TMSOTf, −78° C. DCM, flame-dried 4 Å molecular sieves | Orthoester (29%) |
| 5 | 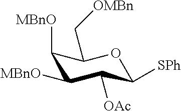 | PhSCl, AgOTf, −78° C. DCM, flame-dried 4 Å molecular sieves | Orthoester (36%) |

After screening several glycosylation conditions, the galactosylated hydroxylysine 6.2 was obtained in 42% yield with only the β-glycoside formed (Table 2). This result not only allowed for the next synthesis step, but also demonstrated the feasibility of stereoselective glycosylation of the ε-amine Boc protected 5-hydroxylysine. To continue the second glycosylation, the PMB deprotection of 6.2 was attempted, to form compound 7. Unfortunately, the PMB deprotection reaction was messy. It is likely that the MBn group was sensitive to oxidants and incompatible with PMB groups (Scheme 3).

TABLE 2
Glycosylation donors and glycosylation conditions tested for the synthesis of
compound 6.2.

| | Glycosylation donor | Condition | Result |
|---|---|---|---|
| 1 | 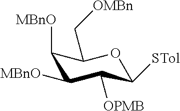 | NIS, Propionitrile −40° C, flame-dried 4 Å molecular sieves | Messy |
| 2 | 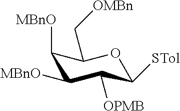 | PhSCl, AgOTf DCM/Propionitrile −78° C., flame-dried 4 Å molecular sieves flame-dried 4 Å molecular sieves | Product (35%) |
| 3 | 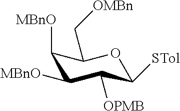 | PhSCl, AgOTf DCM/Propionitrile TTBP, −78° C., flame-dried 4 Å molecular sieves | Product (41%) |
| 4 | 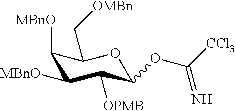 | TMSOTf, DCM/Propionitrile TTBP, −78° C., flame-dried 4 Å molecular sieves | No reaction |
| 5 | 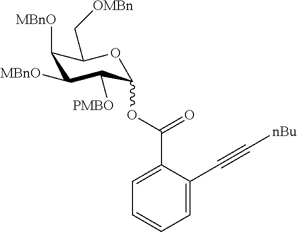 | Ph₃PAuOTf, DCM, TTBP −10° C., flame-dried 4 Å molecular sieves | Messy |
| 6 | 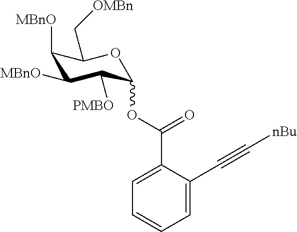 | Ph₃PAuNTf₂, DCM, TTBP −10° C., flame-dried 4 Å molecular sieves | Messy |

With this failure, use of the convergent approach (Scheme 3, Route 2) was explored. The challenge of this route lies in the final β-1,2-trans-glycosylation between the disaccharide and the hydroxylsine building block, considering its steric hindrance and lack of 3-selectivity driving factor plagued with a glycoside at C-2 position. To this end, the 5.1 underwent a hydrolysis to generate a 2-OH free product 5.1a in quantitative yield, which was subjected as the glycosyl acceptor to react with thioglycoside 8 for disaccharide moiety construction. The key point was how to selectively introduce an α-1,2-cis glucosyl bond. Fortunately, when DMF/DCM solvent system and TolSCl/AgOTf initiator³¹ were employed, the desired disaccharide thioglycoside with α-O-glycosyl linkage could be obtained in good yield with exclusive α-stereoselectivity. Subsequently, the disaccharide thioglycoside moiety (9) was successfully glycosylated with the HLBB 4 under AgOTf/PhSCl/TTBP in conditions, and the usage of DCM/CH₃CH₂CN solvent system was the key to induce exclusive β-1,2-trans-glycosylation forming the desired product (10). Next, an Fmoc-compatible ester hydrolysis protocol (LiOH, CaCl₂)) was employed to form the building block (11). This is a convenient and scalable synthetic route, which permitted the synthesis of MBn protected α-D-glucopyranosyl-(1-2)-β-galactopyranosyl (2S,5R)-hydroxylysine building block for the glycopeptide fragment preparation (Scheme 3).

### Example 3: Synthesis of Glycosylated Adiponectin Collagenous Domain (glyACD)

### Material and Methods

### Fmoc-Based Solid-Phase Peptide Synthesis (SPPS)

The solid phase peptide synthesis of peptides/glycopeptides was carried out manually using 2-chlorotrityl resin (CS Biochem, loading: ˜0.5 mmol/g) unless otherwise specified. 2-chloro-trityl chloride resin was swollen in dry CH₂Cl₂ for 30 min then washed with CH₂Cl₂ (5×3 mL). A solution of FmocHN-Xaa-COOH (4.0 equiv. relative to resin capacity) and DIEA (8.0 equiv. relative to resin capacity) in CH₂Cl₂ was added and the resin was shaken at room temperature (r. t.) for 2 h. The resin was washed with DMF (5×3 mL) and CH₂Cl₂ (5×3 mL). The resin was treated with a solution of CH₂Cl₂/CH₃OH/DIEA (17:2:1, v/v/v, 3 mL) for 1 h to cap the unreacted sites, and was washed sequentially with DMF (5×3 mL), CH₂Cl₂ (5×3 mL), and DMF (5×3 mL).

The resin was subsequently submitted to iterative peptide assembly (Fmoc-SPPS). The following Fmoc amino acids and Boc amino acids from GL Biochem were employed: FmocHN-Ala-COOH, FmocHN-Cys(Trt)-COOH, FmocHN-Cys(StBu)-COOH, FmocHN-Asp(OtBu)-COOH, FmocHN-Glu(OtBu)-COOH, FmocHN-Phe-COOH, FmocHN-Gly-COOH, FmocHN-His(Trt)-COOH, FmocHN-Ile-COOH, FmocHN-Lys(Boc)-COOH, FmocHN-Leu-COOH, FmocHN-Met-COOH, FmocHN-Asn(Trt)-COOH, FmocHN-Pro-COOH, FmocHN-Gln(Trt)-COOH, FmocHN-Arg(Pbf)-COOH, FmocHN-Ser(tBu)-COOH, FmocHN-Thr(tBu)-COOH, FmocHN-Val-COOH, FmocHN-Trp(Boc)-COOH, FmocHN-Tyr(tBu)-COOH. For the Fmoc removal step, the resin was treated with the deblock solution (20% piperidine in DMF) at room temperature for 15 min. The resin was then washed sequentially with DMF (5×3 mL), CH₂Cl₂ (5×3 mL), and DMF (5×3 mL). For the coupling step, a solution of Fmoc protected amino acid (2.0 equiv. according to the resin capacity), HATU (2.0 equiv.) and DIEA (5.0 equiv.) in DMF was gently agitated with the resin at room temperature for 40 min. The resin was washed with DMF (5×3 mL), CH₂Cl₂ (5×3 mL), and DMF (5×3 mL). This procedure was repeated twice for coupling each amino acid. For the glycosylated lysine coupling step, a solution of Fmoc protected amino acid (1.5 equiv. according to the resin capacity), HATU (1.5 equiv.) and DIEA (3.0 equiv.) in DMF was gently agitated with the resin at room temperature for 8 h. The resin was washed with DMF (5×3 mL), CH₂Cl₂ (5×3 mL), and DMF (5×3 mL). This procedure was repeated twice.

Cleavage of Crude Protected Peptide Bearing the Free Carboxylic Acid at the C-Terminus from Resin with Cocktail A

The on-resin fully protected peptidyl acid, obtained as described in the previous section, was subjected to mild acidic cleavage cocktail (5-10 mL) of CH₂Cl₂/AcOH/trifluoroethanol (8/1/1, v/v/v), 3 times for 60 min each. Following filtration, the resulting cleavage solutions were combined and concentrated to give crude protected peptide bearing the free carboxylic acid at the C-terminus.

### Results

With the necessary building blocks now available, the total synthesis of glyACD was attempted. The attempt with direct SPPS failed to generate any desired product. After several trials, the synthesis was finalized via ligating two peptide fragments via Ser/Thr ligation³²⁻³⁵ (FIGS. 2A-2C). The 47-amino acid-glycopeptide, hAdn-WM77-b, was prepared using SPPS, followed by TMSOTf treatment to remove the MBn groups present on the glycans, forming hAdn-WM77-b after HPLC isolation (8.9% yield based on the resin loading). Replacement of Bn protecting groups of the disaccharide to MBn protecting groups was important, as all MBn groups could be cleanly removed without cleaving the glycosidic linkage. The resultant glycopeptide underwent deFmoc conditions, which then was, without HPLC purification, directly ligated with peptide salicylaldehyde ester, hAdn-WM-a, under Ser/Thr ligation conditions in 30.8% yield over 2 steps after HPLC isolation. With the same strategy, the synthesis of all possible glycoforms of glyACD (15 in total) was successfully completed (FIG. 2D).

### Example 4: Characterization of the Anti-Proliferative Activity of the glyACD Peptides

### Material and Methods

### Cancer Cell Proliferative Assay

MDA-MB-231 cells were cultured in DMEM supplemented with 10% FBS and 1% penicillin-streptomycin-fungisone at 37° C. and under 5% CO₂ 95% humidified air. After harvesting, cells were seeded at a density of 3000 cells per well in 96-well plates and then cultured for 24 hours. After fasting in DMEM with 0.5% FBS for 24 hours, cells were subsequently stimulated with 2.5% FBS in the presence of peptides, human adiponectin or other drugs. The viable cell numbers at 24 hours at different doses were manually counted by mixing with trypan blue dye for analysis. The maximum inhibition rate (Emax) and half maximal effective concentration (EC50) were used to evaluate the anti-tumor activity of peptides and adiponectin.

### Combination Index (CI) Analysis

CI was used to determine the mode of drug interaction. To analyze the CI, MDA-MB-31 cells were seeded in 96-well plates to do the chess-board assay. Cells were treated with a concentration of adiponectin ranging from 0 to 2.5 μg/ml and of hAdn-WM or hAdn-WM656877101 ranging from 0 to 20 μg/ml for 24 hours and then cell numbers were counted. CI was calculated by the formula of CI=CA,x/Cx,A+CB,x/Cx,B, where CA,x and CB,x are the concentration of drug A and B used in combination to achieve x % drug effect (inhibition rate); Cx,A and Cx,B are the concentrations for single agents to achieve the same effect. A CI of less than, equal to, and more than 1 indicates synergy, additivity, and antagonism, respectively.

### Statistical Analysis

Significant differences between groups were analyzed by t-test or two-way ANOVA (GraphPad Prism 8.0.2 Software, Inc., San Diego, CA, USA).

### Results

Human breast cancer MDA-MB-231 cells were used to evaluate the anti-proliferative activities of adiponectin, and the 15 glyACD and non-glycosylated hAdn-WM peptides. After 24-hours of incubation, the cell number was manually counted to calculate the maximum inhibition rate (Eₘₐₓ) and the half-maximal effective concentration (EC₅₀) (Table 3). When compared to full-length human adiponectin (hadiponectin), hAdn-WM exhibited a significantly reduced Eₘₐₓ (by ˜three-fold) and a much higher EC₅₀ (by ˜19-fold). The Eₘₐₓ of glyACD ranged from ˜26.0% to ˜53%, which was lower than that of full-length adiponectin but higher than hAdn-WM. Among all glyACD, hAdn-WM656877101 with tetra-glycans exhibited the lowest EC₅₀ (Table 3).

TABLE 3
Comparison of the Eₘₐₓ and EC₅₀ for the anti-proliferative
activity of glyACD in human breast cancer MDA-MB-231 cells.
For each peptide, the number in the peptide name (e.g., 65,
68, 77, 101) indicates the glycosylated lysine residue(s).

| Peptides | Eₘₐₓ (%) | EC₅₀ (μg/mL) |
|---|---|---|
| hAdn-WM | 23.0 ± 4.23 | 6.27 ± 0.62 |
| hAdn-WM 65 | 39.3 ± 5.05 | 2.00 ± 0.89 |
| hAdn-WM 68 | 38.3 ± 2.40 | 1.84 ± 0.13 |
| hAdn-WM 77 | 33.9 ± 2.59 | 1.26 ± 0.12 |
| hAdn-WM 101 | 32.1 ± 5.05 | 1.78 ± 0.76 |
| hAdn-WM6568 | 31.0 ± 4.66 | 1.46 ± 0.81 |
| hAdn-WM6577 | 35.5 ± 1.95 | 1.19 ± 0.68 |
| hAdn-WM65101 | 36.3 ± 3.81 | 1.86 ± 0.58 |
| hAdn-WM6877 | 38.5 ± 3.75 | 1.33 ± 0.10 |
| hAdn-WM68101 | 35.2 ± 2.63 | 1.55 ± 0.72 |
| hAdn-WM77101 | 34.2 ± 3.72 | 2.04 ± 0.71 |
| hAdn-WM656877 | 37.7 ± 6.2 | 1.58 ± 0.31 |
| hAdn-WM6568101 | 38.4 ± 0.32 | 2.42 ± 0.49 |
| hAdn-WM6577101 | 42.9 ± 6.53 | 1.45 ± 0.24 |
| hAdn-WM6877101 | 45.0 ± 3.46 | 1.71 ± 0.07 |
| hAdn-WM656877101 | 48.7 ± 4.12 | 0.78 ± 0.35 |
| hAdiponectin | 69.3 ± 3.34 | 0.33 ± 0.11 |

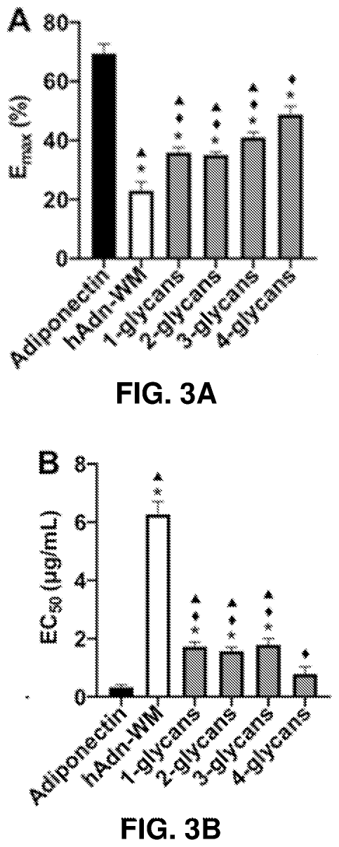

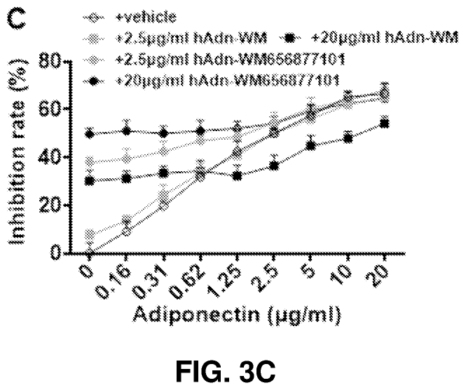

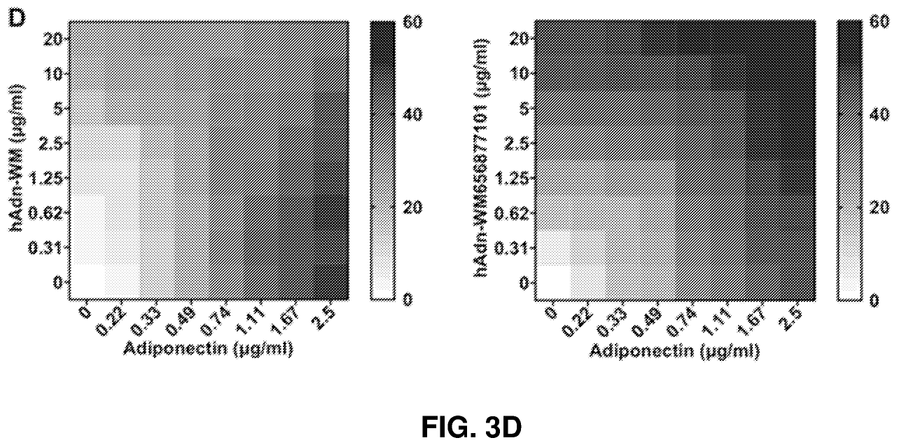

The glyACD with mono-, di-, and tri-glycans exhibited lower Eₘₐₓ than that of hAdn-WM656877101, which contained tetra-glycans (FIG. 3A). There were no significant differences between the EC₅₀ of glyACD containing mono-, di- and tri-glycans, which were all significantly higher than that of hAdn-WM656877101 (FIG. 3B). In the presence of 20 μg/ml hAdn-WM, the Eₘₐₓ of human adiponectin significantly decreased (66.7±4.29% vs 54.2±2.95%%, P<0.05). In contrast, hAdn-WM656877101 did not inhibit the Eₘₐₓ of human adiponectin (FIG. 3C). In the chessboard assay, co-incubation with 5, 10, or 20 μg/ml of hAdn-WM reduced the anti-proliferative activity of adiponectin at a concentration from 0.74 to 2.5 μg/ml, (FIG. 3D), indicating antagonistic effects (combination index >1). In contrast, co-incubation with 2.5, 5, 10, or 20 μg/ml of hAdn-WM656877101 enhanced the anti-proliferative activity of adiponectin at a concentration lower than 2.5 μg/ml (FIG. 3D), indicating that the combination generated more than additive effects (combination index <1). In human patients, there are reduced full-length adiponectin levels but not the complete absence of this molecule, thus the more than additive effects are beneficial. It is useful for the peptide mimetics to not compete with or inhibit the effects of the endogenous full-length adiponectin.

### Example 5: The hAdn-WM6877 glyACD Exhibits Anti-Breast Cancer Activity

### Material and Methods

### Animals

Mice with (WT) or without (AKO) wild type ADIPOQ alleles were maintained on both C57BL/6J and FVB/N background. In metabolic related assays, WT and AKO of C57BL/6J background were fed with high-fat diet (19.33 kcal/g from 49.85% fat, 20% protein, and 30.15% carbohydrate; D12451; Research Diet, New Brunswick, NJ, U.S.A.) to induce dietary obesity. For studies related to mammary tumor development, FVB/N-Tg (MMTV-PyVT)634 Mul/J [002374 from Jackson Laboratory (Bar Harbor, ME, U.S.A.)] were cross-bred with AKO of FVB/N background to produce mice with (PyVT-WT) or without (PyVT-AKO) the ADIPOQ alleles.

### In Vivo Anti-Tumor Activity Assay

All animal care and experimental protocols complied with the institutional guidelines for the care and use of laboratory animals and were approved by the Committee on the Use of Live Animals for Teaching and Research of the University of Hong Kong. All mouse models were housed in a room under the controlled temperature (23±1° C.) and 12-h light-dark cycles, with free access to water and standard mouse chow if not specifically noted (4.07 kcal/g; LabDiet 5053; LabDiet, Purina Mills, Richmond, VA, U.S.A.).

Human breast cancer MDA-MB-231 cells were treated with phosphate buffer saline (PBS) or 20 μg/ml adiponectin glycol-peptides for 24 hours in the absence of fetal bovine serum. A total number of 2×10⁵ cells were harvested from each treatment group and injected into the right third mammary fat pad of NOD/SCID mice (six-weeks old, female) and a total number of 5×10⁶ cells were harvested from each treatment group and injected in to the right third mammary fat pad of Nude mice (six-weeks old, female). Tumor development was monitored 3 times a week by a digital Vernier caliper and tumor volume was calculate using the formula [sagittal dimension (mm)×cross dimension (mm)×2]/2. Mice were sacrificed thirteen days after injection for collecting and weighing tumors and lungs. Data are presented as mean±SEM; *, P<0.05, **, P<0.01 vs corresponding vehicle controls (n=6).

FVB/N-Tg (MMTV-PyVT) 634 Mul/J (FVB/N pyvt+/−) adiponectin knocks out (AKO) mice were intraperitoneally injected with 40 g ACD peptide hAdn-WM6877 (0.4 μg/l, 100 μl) or equal volume of PBS per day starting from 7-week age. Tumor development was monitored every week. Tumor volume was measured using a digital Vernier caliper by the formula [sagittal dimension (mm)×cross dimension (mm)×2]/2. Mice were sacrificed at the end of treatment for collecting and weighing tumors and lungs. Data are presented as mean±SEM; *, P<0.05, **, P<0.01 vs corresponding vehicle controls (n=6).

### Results

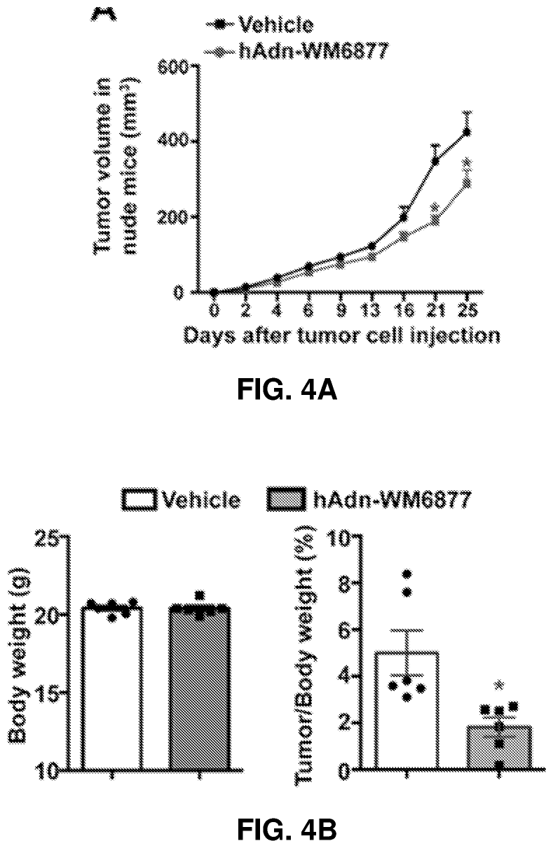

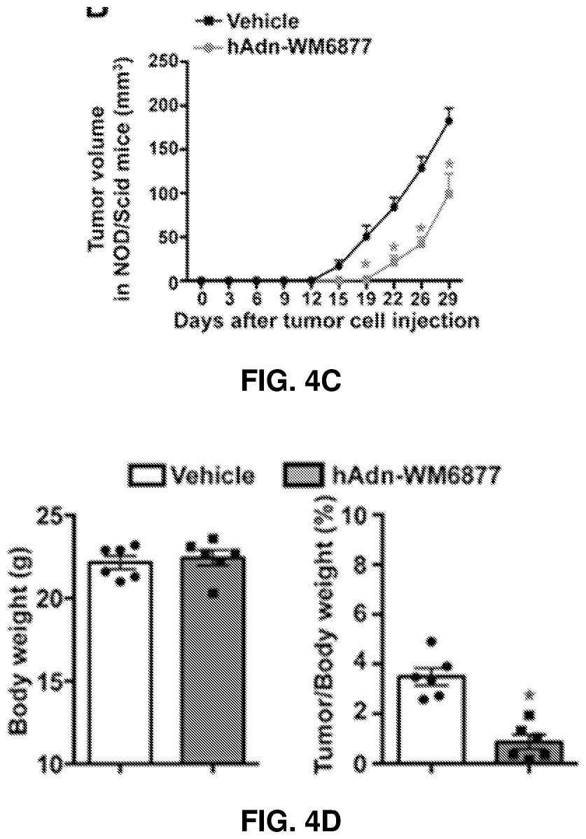

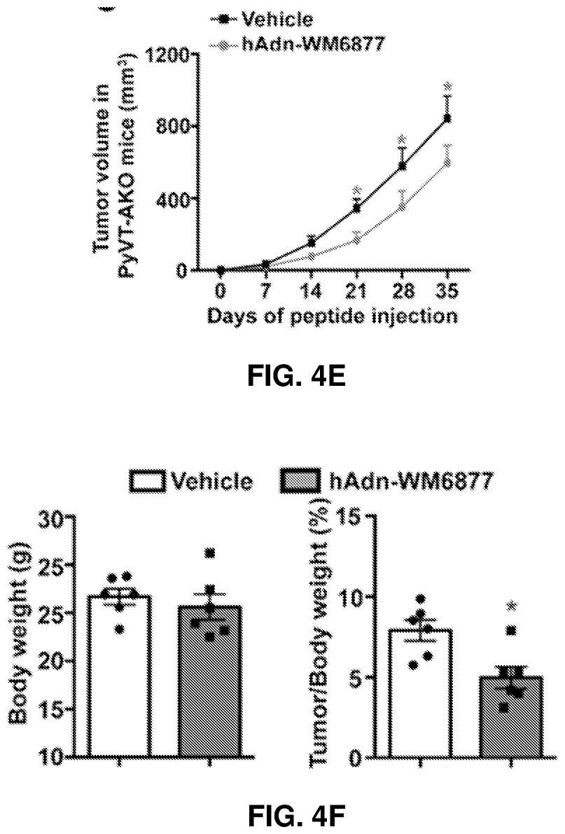

The di-glycan peptide hAdn-WM6877 was selected and synthesized in larger amounts (20 mg) to examine its anti-breast cancer activity. First, MDA-MB-231 cells treated with phosphate buffered saline (PBS) or hAdn-WM6877 for 24 hours were implanted orthotopically into the third right mammary fat pad of athymic nude (FIGS. 4A-4B) or NOD/Scid mice (FIGS. 4C-4D). The development of mammary tumors was monitored on a regular basis. Compared to the vehicle group, tumor development was significantly attenuated in mice implanted with MDA-MB-231 cells pretreated with hAdn-WM6877 (FIGS. 4A-4D). There were no significant differences in body weight between the vehicle and treatment groups. However, the percentage tumor weight was significantly decreased in both nude and NOD/Scid mice implanted with MDA-MB-231 cells pre-treated with hAdn-WM6877 (FIGS. 4B and 4D).

The transgenic MMTV PyVT mice lacking the Adipoq alleles (PyVT-AKO) exhibit spontaneous mammary tumor development starting from the age of seven or eight weeks³⁶. The PBS or hAdn-WM6877 (40 μg/mouse/day) was intraperitoneally injected into PyVT-AKO mice from the age of eight weeks. Tumor development was monitored on a weekly basis. Treatment with hAdn-WM6877 significantly inhibited mammary tumor development in PyVT-AKO mice (FIG. 4E). After five-weeks of treatment, tumors were collected for examination. When compared to the vehicle group, the tumor-to-body weight ratios were significantly decreased (by over two-fold) in PyVT-AKO mice treated with hAdn-WM6877 (FIG. 4F). Collectively, the above results demonstrate that the di-glycan peptide hAdn-WM6877 elicited potent anti-breast cancer activity in mouse models.

### Example 6: hAdn-WM6877 glyACD Improves Glucose and Insulin Tolerance

### Material and Methods

### Metabolic Regulative Effect of ACD Peptide

ACD peptide, hAdn-WM6877, was tested for anti-obesity and insulin-sensitizing functions. WM6877 (40 μg, 0.4 μg/l, 100 μl) or an equal volume of PBS was injected into 12-week old C₅₇BL/6J AKO mice every day for 5 weeks. These mice had been fed with high-fat diet for 8 weeks to induce dietary obesity. Body weight and fat mass composition were measured every week for mice that were either starved overnight or fed ad libitum. The body mass composition was assessed using a Bruker minispec Body Composition Analyzer (Bruker Optics, Inc., Woodlands, TX) and all the mice were conscious and unanesthetized. Blood glucose was measured by tail nicking using an Accu-Check Advantage II Glucometer (Roche Diagnostics, Mannheim, Germany). Circulating and tissue contents of lipids, including triglycerides, total cholesterols, were analyzed using LiquiColor Triglycerides and Stanbio Cholesterol (Stanbio Laboratory, Boerne, TX) and the Half-Micro Test Kit (Roche Diagnostics), respectively. Metabolic rate (VO₂, VCO₂, and respiratory exchange ratio [RER]) was measured by indirect calorimetry using a six-chamber open-circuit Oxymax system component of the Comprehensive Laboratory Animal Monitoring System (CLAMS; Columbus Instruments, Columbus, OH). Before recording the data, all mice were acclimatized to the cage for 48 hours.

### Histological Assay

After injection of hAdn-WM6877 for 4 weeks, AKO mice were sacrificed to collect liver tissues. After being cut into small pieces, tissues were fixed in 10% formalin solution for 48 hours and then transferred to 75% ethanol for long term storage at 4° C. After being frozen, liver tissues were embedded in Tissue-Tek OCT compound (Sakura® Finetek, CA, U.S.A.), sectored at 5 μm, then stained with Oil Red O (Sigma-Aldrich), and incubated for 10 minutes. The adipocytes were captured and by Image J software (Version 1.51, NIH, USA). The fields were randomly chosen to presented adipocytes. All slides were examined under Olympus biological microscope BX41, and images were captured using an Olympus DP72 color digital camera.

### Quantitative PCR (QPCR) Analysis

After 8 weeks of HFD, 40 μg ACD peptide hAdn-WM6877 (0.4 μg/l, 100 μl) or equal volume of PBS was daily injected into AKO mice. Mice were sacrificed after 5 weeks of treatment and liver samples were collected. Total RNA was isolated from liver samples using TRIZOL reagent according to the manufacturer's instructions. Approximately 100 mg were homogenized in 1 ml TRIZOL reagent. After centrifugation (12,000×g), supernatants were collected to remove insoluble materials. 200 microliters of chloroform was then added into the homogenate, followed by vigorous shaking and incubation at room temperature for 5 minutes. The mixture was then centrifuged (12,000×g) for ten minutes at 4° C. After centrifugation, 500 μl of isopropanol were added to the supernatant. After centrifugation (12,000×g) for 10 minutes at 4° C., 75% ethanol was used to wash the precipitated RNA pellets and then the RNA was dissolved in DEPC water. The concentration of RNA was determined by Gene Quant RNA/DNA calculator at absorbance of 260/280 nm (Pharmacia Biotech, Uppsala, Sweden). After the preparation of samples, QPCR was performed using SYBR Green PCR Master Mix on an ABI PRISM 7900 HT Sequence Detection System (Applied Biosystems, Foster City, CA). The levels of ribosomal 18S, IL6, IL10, CD68, MCP-1 and TNFα genes were detected. The primers used were purchased from RIBOBIO. Quantification was achieved by AA Ct values that were normalized with GADPH as a reference control. The expression levels were calculated by 2-AA Ct for comparison.

### Results

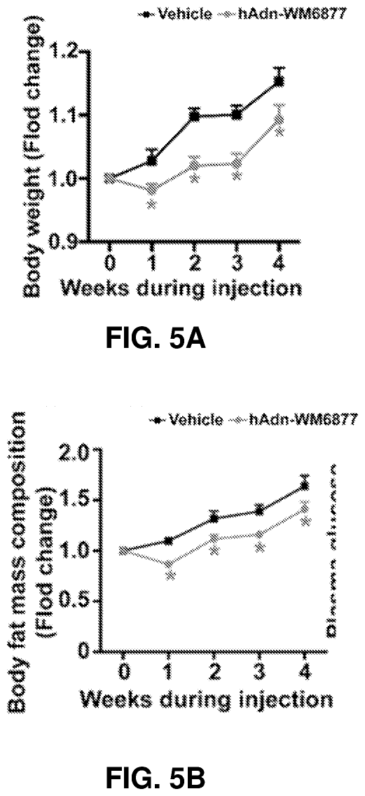

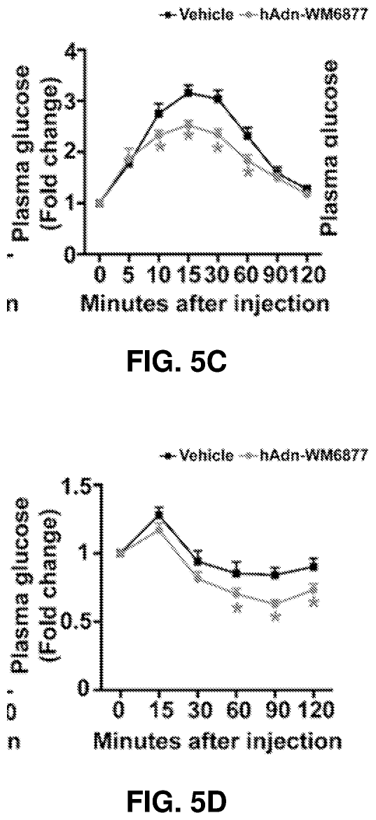

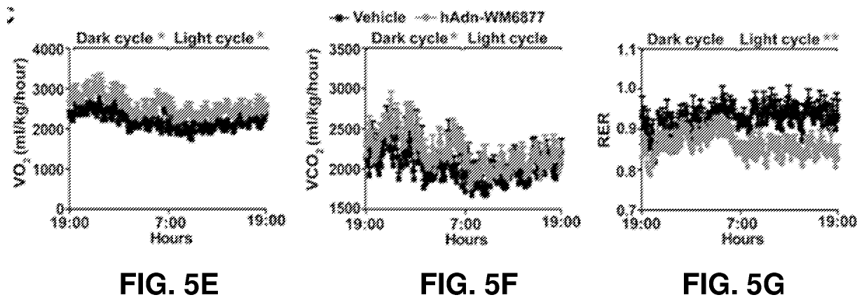

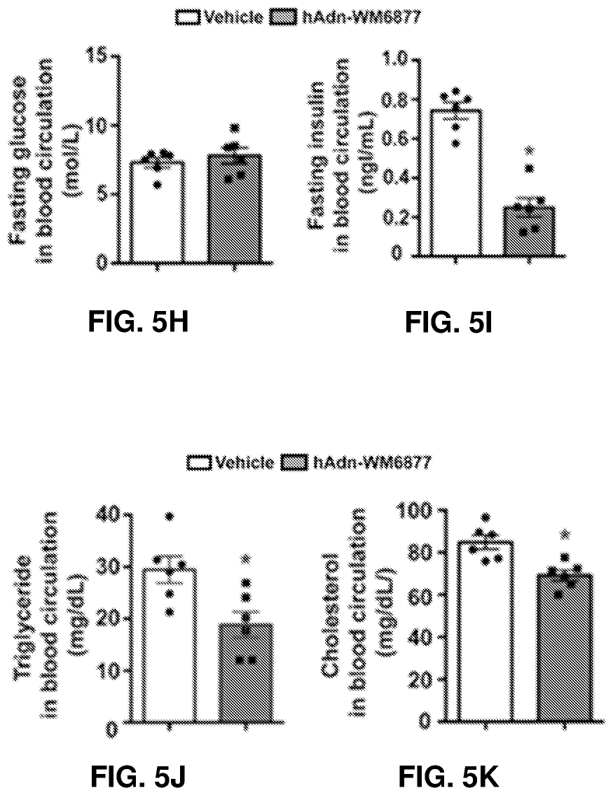

Adiponectin knockout (AKO) mice on the C57BL/6J background were used to study the metabolic functions of hAdn-WM6877. AKO mice were given a high-fat diet (HFD) for 12 weeks, starting from the age of four weeks. PBS or hAdn-WM6877 peptide (40 μg/mouse/day) was administered intraperitoneally during the last four-weeks of HFD treatment. Compared to the vehicle control group, daily injection with hAdn-WM6877 significantly attenuated the gain of body weight and body fat mass (FIGS. 5A-5B). After four weeks of injection, mice treated with hAdn-WM6877 exhibited significantly improved glucose and insulin tolerance (FIG. 5C-5D).

The metabolic performance was evaluated by the Comprehensive Laboratory Animal Monitoring System (CLAMS). Compared to those of the vehicle controls, treatment with hAdn-WM6877 significantly increased oxygen consumption (VO₂) during both dark (2345.0±102.9 vs 2689.1±142.2 ml/kg/hour, P<0.05) and light (2070.7±33.6 vs 2416.9±142.7 ml/kg/hour, P<0.05) cycles (FIG. 5D), and increased carbon dioxide release (VCO₂) during dark (2077.1±68.0 vs 2320.2±74.1 mL/kg/hour, *, P<0.05) cycles (FIG. 5E). As a result, the respiratory exchange ratio (RER) during the light cycle was significantly lower in mice treated with hAdn-WM6877 than those of the vehicle controls (FIG. 5F) (0.84±0.04 vs 0.92±0.02, P<0.01). The glycosylated peptide increases the metabolic rate and burn up more fat in obese patients, thus improving the energy homeostasis. The fasting serum levels of triglyceride and total cholesterol were also significantly decreased in mice treated with hAdn-WM6877 (FIGS. 5J-5K).

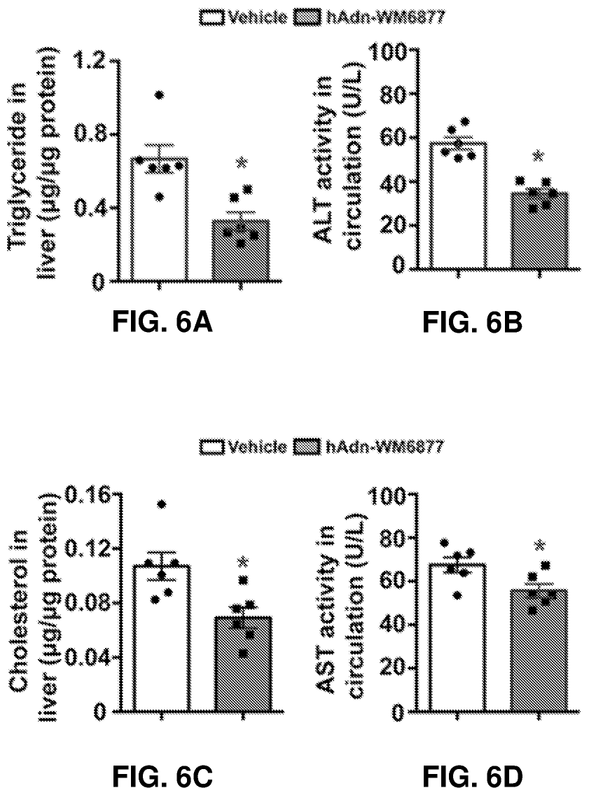

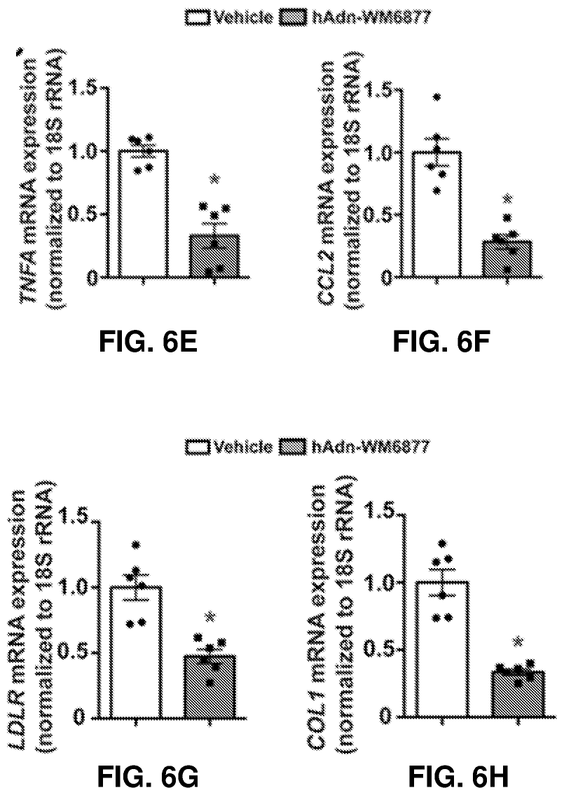

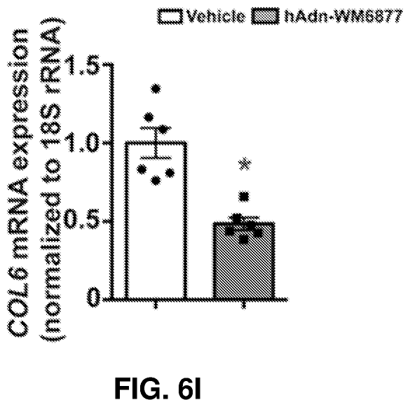

H&E and Oil Red O staining revealed that daily treatment with hAdn-WM6877 significantly reduced the lipid accumulation in livers of HFD-fed AKO mice. The triglyceride and cholesterol content were decreased by ˜51.9% and ˜35.4% in livers of mice treated with hAdn-WM6877 as compared to the vehicle control animals (FIGS. 6A, 6C). The circulating levels of liver injury markers, alanine transaminase (ALT) and aspartate aminotransferase (AST), were both significantly decreased by treatment with hAdn-WM6877 (FIGS. 6B, 6D). Compared to the vehicle control animals, the mRNA expression levels of steatohepatitis-associated genes, including those encoding tumor necrosis factor alpha (TNFA), monocyte chemoattractant protein-1 (CCL2), low-density lipoprotein receptor (LDLR), collagen type I (COL1) and VI (COL6) were significantly decreased in livers of mice treated with hAdn-WM6877 (FIGS. 6E-61). In summary, the results indicate that hAdn-WM6877 mimicked adiponectin to elicit insulin-sensitizing, anti-inflammatory, and hepatoprotective functions in AKO mice challenged with HFD.

### Discussion

Adiponectin is a circulating hormone produced abundantly from adipose tissue and has therapeutic potential for metabolic, cancer and cardiovascular diseases. Biochemical and pharmacological studies of the human adiponectin correlating the domain structure to function have been restricted by protein heterogeneity and difficulty in obtaining the homogeneous collagenous domain with site-specific modification(s). In this study an accessible and scalable route for synthesis of the glycosylated adiponectin collagenous domain using stereoselective glycan synthesis and chemical peptide ligation was developed, leading to 15 homogeneously glycosylated variants of ACD for the first time. Subsequently, the biological activities and pharmacological properties of the glycosylated adiponectin peptides were evaluated and compared with the full-length human adiponectin. The results demonstrated that the glycan plays an important role of the collagenous domain of adiponectin in the inhibition of cancer cell growth as well as the regulation of systemic energy metabolism.

A key feature of this work is the power of chemical synthesis of glycosylated adiponectin collagen domain in systematically addressing the role of glycosylation on activity and specificity. This study not only shows how chemistry facilitates the solution to the unsolved biological challenges associated with posttranslational glycosylation at a molecular level, but also paves the way for total synthesis of glycosylated full-length human adiponectin for further study. Importantly, the in vivo studies showed that hAdn-WM6877 exhibited robust anti-tumor activity, enhanced fatty acid consumption, improved glucose tolerance and insulin sensitivity, which opens the door to explore the opportunity of using the synthetic glycopeptide as a potential adiponectin downsized mimic supplementary in metabolic diseases treatment, and the knowledge in adiponectin glycosylation might trigger the development of new drug and mechanism of study.

### REFERENCES

1. Scherer, P. E., Williams, S., Fogliano, M., Baldini, G. & Lodish, H. F. A novel serum protein similar to C1q, produced exclusively in adipocytes. J. Biol. Chem. 270, 26746-26749 (1995).
2. Hu, E., Liang, P. & Spiegelman, B. M. AdipoQ is a novel adipose-specific gene dysregulated in obesity. J. Biol. Chem. 271, 10697-10703 (1996).
3. Nakano, Y. et al. Isolation and characterization of GBP28, a novel gelatin-binding protein purified from human plasma. J. Biol. Chem. 120, 803-812 (1996).
4. Maeda, K. et al. cDNA cloning and expression of a novel adipose specific collagen-like factor, apM1 (adipose most Abundant gene transcript 1). Biochem. Biophys. Res. Commun. 221, 286-289 (1996).
5. Kadowaki, T. et al. Adiponectin and adiponectin receptors in insulin resistance, diabetes, and the metabolic syndrome. J. Clin. Invest. 116, 1784-1792 (2006).
6. Achari, A. E. & Jain, S. K. Adiponectin, a therapeutic target for obesity, diabetes, and endothelial dysfunction. Int. J. Mol. Sci. 18, 1321-1337 (2017).
7. Goldstein, B. J., Scalia, R. G. & Ma, X. L. Protective vascular and myocardial effects of adiponectin. Nature Reviews Cardiology. 6, 27-35 (2009).
8. Li, S., Shin, H. J., Ding, E. L. & Dam, R. M. V. Adiponectin levels and risk of type 2 diabetes: a systematic review and meta-analysis. JAMA. 302, 179-188 (2009).
9. Kim, D. H., Kim, C., Ding, E. L., Townsend, M. K. & Lipsitz, L. A. Adiponectin levels and the risk of hypertension: a systematic review and meta-analysis. Hypertension. 62, 27-32 (2013).
10. Dalamaga, M., Diakopoulos, K. N. & Mantzoros, C. S. The role of adiponectin in cancer: a review of current evidence. Emdocr. Rev. 33, 547-594 (2012).
11. Liu, Y., Vu, V. & Sweeney, G. Examining the potential of developing and implementing use of adiponectin-targeted therapeutics for metabolic and cardiovascular diseases. Front. Endocrinol. 10, 842 (2019).
12. Wang, Y., Xu, A., Knight, C., Xu, L. Y. & Cooper, G. J. S. Hydroxylation and glycosylation of the four conserved lysine residues in the collagenous domain of adiponectin. Potential role in the modulation of its insulin-sensitizing activity. J. Biol. Chem. 277, 19521-19529 (2002).
13. Wang, Y. et al. Proteomic and functional characterization of endogenous adiponectin purified from fetal bovine serum. Proteomics, 4, 3933-3942 (2004).
14. Yamauchi, T et al. The fat-derived hormone adiponectin reverses insulin resistance associated with both lipoatrophy and obesity. Nat. Med. 7, 941-946 (2001).
15. Berg, A. H., Combs, T. P., Du, X., Brownlee, M. & Scherer, P. E. The adipocyte-secreted protein Acrp30 enhances hepatic insulin action. Nat. Med. 7, 947-953 (2001).
16. Kim, Y. et al. The adiponectin receptor agonist AdipoRon ameliorates diabetic nephropathy in a model of type 2 diabetes. J. Am. Soc. Nephrol. 29, 1108-1127 (2018).
17. Abou-Samra, M., Selvais, C. M., Dubuisson, N. & Brichard, S. M. Adiponectin and its mimics on skeletal muscle: insulin sensitizers, fat burners, exercise mimickers, muscling pills or everything together. Int. J. Mol. Sci. 21, 2620-2640 (2020).
18. Sayeed, M. et al. A collagen domain-derived short adiponectin peptide activates APPL1 and AMPK signaling pathways and improves glucose and fatty acid metabolisms. J. Biol. Chem. 293, 13509-13523 (2018).
19. Okada-Iwabu, M. et al. A small-molecule AdipoR agonist for type 2 diabetes and short life in obesity. Nature. 503, 493-499 (2013).
20. Fruebis, J. et al. Proteolytic cleavage product of 30-kDa adipocyte complement-related protein increases fatty acid oxidation in muscle and causes weight loss in mice. Proc. Natl. Acad. Sci. U.S.A 98, 2005-2010 (2001).
21. Shapiro, L. & Scherer, P. E. Curr. Biol. 8, 335-338 (1998).
22. Yamauchi, T. et al. Cloning of adiponectin receptors that mediate antidiabetic metabolic effects. Nature. 423, 762-769 (2003).
23. Wang, Y., Lam, K., Yau, M. & Xu, A. Post-translational modifications of adiponectin: mechanisms and functional implications. Biochem. J. 409, 623-633 (2008).
24. Peake, P. W., Hughes, J. T., Shen, Y. & Charlesworth, J. A. Glycosylation of human adiponectin affects its conformation and stability. J. Mol. Endocrinol. 39, 45-52 (2007).
25. Richards, A. A. et al. Adiponectin multimerization is dependent on conserved lysines in the collagenous domain: evidence for regulation of multimerization by alterations in posttranslational modifications. Mol. Endocrinol. 20, 1673-1687 (2006).
26. Marin, J., Didierjean, C., Briand, A. A. & Guichard, G. Diastereoselective hydroxylation of 6-Substituted piperidin-2-ones: an efficient synthesis of (2S,5R)-5-hydroxylysine and related α-Amino Acids. J. Org. Chem. 67, 8440-8449 (2002).
27. Adamczyk, M., Johnson, D. D. & Reddy, R. E. Collagen cross-links: Synthesis of pyridinoline, deoxypyridinoline and their analogues. Tetrahedron. 55, 63-88 (1999).
28. Herbert, K. R. Williams, G. M. Cooper, G. J. S. & Brimble, M. A. Synthesis of glycosylated 5-hydroxylysine, an important amino acid present in collagen-like proteins such as adiponectin. Org. Biomol. Chem. 10, 1137-1144 (2012).
29. Fukuyama, T. Lin, S. & Li, L. Facile reduction of ethyl thiol esters to aldehydes: application to a total synthesis of (+)-neothramycin a methyl ether. J. Am. Chem. Soc. 112, 7050-7051 (1990).
30. Scholl, M., Trnka, T. M., Morgan, J. P. & Grubbs. Increased ring closing metathesis activity of ruthenium-based olefin metathesis catalysts coordinated with imidazolin-2-ylidene ligands. Tetrahedron. Lett. 40, 2247-2250 (1999).
31. Lu, S., Lai, Y., Chen, J., Liu, C. & Mong, K. T. Dimethylformamide: an unusual glycosylation modulator. Angew. Chem. Int. Ed. 50, 7315-7320 (2011).
32. Zhang, Y., Xu, C., Lam, H. Y., Lee, C. L. & Li, X. Protein chemical synthesis by serine and threonine ligation. Proc. Natl. Acad. Sci. U.S.A 110, 6657-6662 (2013).
33. Lee, C. L., Liu, H., Wong, C. T. T., Chow, H. Y. & Li, X. Enabling N-to-C Ser/Thr ligation for convergent protein synthesis via combining chemical ligation approaches. J. Am. Chem. Soc. 138, 10477-10484 (2016).
34. Tan, Y., Wei, T., Wu, H. & Li, X. Chemical protein synthesis: advances, challenges and outlooks. J. Am. Chem. Soc. 142, 20288 (2020).
35. Liu, H. & Li, X. Serine/Threonine ligation: origin, mechanistic aspects, and applications. Acc. Chem. Res. 51, 1643-1655 (2018).
36. Lam, J. B. B. et al. Adiponectin Haploinsufficiency Promotes Mammary Tumor Development in MMTV-PyVT Mice by Modulation of Phosphatase and Tensin Homolog Activities. PLoS One. 4, 4968 (2009).
37. Achari, AE. and Jain S K. Adiponectin, a Therapeutic Target for Obesity, Diabetes, and Endothelial Dysfunction. Int J Mol Sci. 18(6):1321 (2017).
38. Robinson, K., et al. Clinical review: Adiponectin biology and its role in inflammation and critical illness. Crit Care. 15(2): 221 (2011).

It is understood that the disclosed methods and compositions are not limited to the particular methodology, protocols, and reagents described as these may vary. It is also to be understood that the terminology used herein is for the purpose of describing particular embodiments only, and is not intended to limit the scope of the present invention which will be limited only by the appended claims.

Disclosed are materials, compositions, and components that can be used for, can be used in conjunction with, can be used in preparation for, or are products of the disclosed methods and compositions. These and other materials are disclosed herein, and it is understood that when combinations, subsets, interactions, groups, etc. of these materials are disclosed, that while specific reference of each various individual and collective combinations and permutation of these compounds may not be explicitly disclosed, each is specifically contemplated and described herein. For example, if a step is disclosed and discussed and a number of modifications that can be made to a number of components including the step are discussed, each and every combination and permutation of step and the modifications that are possible are specifically contemplated unless specifically indicated to the contrary. Thus, if a class of molecules A, B, and C are disclosed as well as a class of molecules D, E, and F and an example of a combination molecule, A-D is disclosed, then even if each is not individually recited, each is individually and collectively contemplated. Thus, is this example, each of the combinations A-E, A-F, B-D, B-E, B-F, C-D, C-E, and C-F are specifically contemplated and should be considered disclosed from disclosure of A, B, and C; D, E, and F; and the example combination A-D. Likewise, any subset or combination of these is also specifically contemplated and disclosed. Thus, for example, the sub-group of A-E, B-F, and C-E are specifically contemplated and should be considered disclosed from disclosure of A, B, and C; D, E, and F; and the example combination A-D. Further, each of the materials, compositions, components, etc. contemplated and disclosed as above can also be specifically and independently included or excluded from any group, subgroup, list, set, etc. of such materials. These concepts apply to all aspects of this application including, but not limited to, steps in algorithms or methods of making and using the disclosed compositions. Thus, if there are a variety of additional steps that can be performed it is understood that each of these additional steps can be performed with any specific embodiment or combination of embodiments of the disclosed methods, and that each such combination is specifically contemplated and should be considered disclosed.

It must be noted that as used herein and in the appended claims, the singular forms “a,” “an,” and “the” include plural reference unless the context clearly dictates otherwise.

Throughout the description and claims of this specification, the word “comprise” and variations of the word, such as “comprising” and “comprises,” means “including but not limited to,” and is not intended to exclude, for example, other additives, components, integers or steps.

“Optional” or “optionally” means that the subsequently described event, circumstance, or material may or may not occur or be present, and that the description includes instances where the event, circumstance, or material occurs or is present and instances where it does not occur or is not present.

Unless the context clearly indicates otherwise, use of the word “can” indicates an option or capability of the object or condition referred to. Generally, use of “can” in this way is meant to positively state the option or capability while also leaving open that the option or capability could be absent in other forms or embodiments of the object or condition referred to. Unless the context clearly indicates otherwise, use of the word “may” indicates an option or capability of the object or condition referred to. Generally, use of “may” in this way is meant to positively state the option or capability while also leaving open that the option or capability could be absent in other forms or embodiments of the object or condition referred to. Unless the context clearly indicates otherwise, use of “may” herein does not refer to an unknown or doubtful feature of an object or condition.

Ranges may be expressed herein as from “about” one particular value, and/or to “about” another particular value. When such a range is expressed, also specifically contemplated and considered disclosed is the range from the one particular value and/or to the other particular value unless the context specifically indicates otherwise. Similarly, when values are expressed as approximations, by use of the antecedent “about,” it will be understood that the particular value forms another, specifically contemplated embodiment that should be considered disclosed unless the context specifically indicates otherwise. It will be further understood that the endpoints of each of the ranges are significant both in relation to the other endpoint, and independently of the other endpoint unless the context specifically indicates otherwise. It should be understood that all of the individual values and sub-ranges of values contained within an explicitly disclosed range are also specifically contemplated and should be considered disclosed unless the context specifically indicates otherwise. Finally, it should be understood that all ranges refer both to the recited range as a range and as a collection of individual numbers from and including the first endpoint to and including the second endpoint. In the latter case, it should be understood that any of the individual numbers can be selected as one form of the quantity, value, or feature to which the range refers. In this way, a range describes a set of numbers or values from and including the first endpoint to and including the second endpoint from which a single member of the set (i.e. a single number) can be selected as the quantity, value, or feature to which the range refers. The foregoing applies regardless of whether in particular cases some or all of these embodiments are explicitly disclosed.

Unless defined otherwise, all technical and scientific terms used herein have the same meanings as commonly understood by one of skill in the art to which the disclosed method and compositions belong. Although any methods and materials similar or equivalent to those described herein can be used in the practice or testing of the present method and compositions, the particularly useful methods, devices, and materials are as described. Publications cited herein and the material for which they are cited are hereby specifically incorporated by reference. Nothing herein is to be construed as an admission that the present invention is not entitled to antedate such disclosure by virtue of prior invention. No admission is made that any reference constitutes prior art. The discussion of references states what their authors assert, and applicants reserve the right to challenge the accuracy and pertinency of the cited documents. It will be clearly understood that, although a number of publications are referred to herein, such reference does not constitute an admission that any of these documents forms part of the common general knowledge in the art.

Although the description of materials, compositions, components, steps, techniques, etc. may include numerous options and alternatives, this should not be construed as, and is not an admission that, such options and alternatives are equivalent to each other or, in particular, are obvious alternatives.

Every composition disclosed herein is intended to be and should be considered to be specifically disclosed herein. Further, every subgroup that can be identified within this disclosure is intended to be and should be considered to be specifically disclosed herein. As a result, it is specifically contemplated that any composition, or subgroup of compositions can be either specifically included for or excluded from use or included in or excluded from a list of compositions.

Those skilled in the art will recognize, or be able to ascertain using no more than routine experimentation, many equivalents to the specific embodiments of the method and compositions described herein. Such equivalents are intended to be encompassed by the following claims.

1. A method of making an amino acid building block of Formula (I): 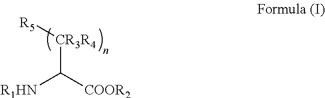
wherein n is an integer from 0 to 4;
wherein each of R₁ and R₂ is independently a protecting group;
wherein each occurrence of R₃ and R₄ is independently a hydrogen, a substituted alkyl, an unsubstituted alkyl, a hydroxyl, a thiol, or NR₁₃R₁₄, each of R₁₃ and R₁₄ is hydrogen, a substituted alkyl, or an unsubstituted alkyl;
wherein R₅ is absent, a ketimine, an imidazole, an amino group, a carboxyl, a carboxylate, a hydroxyl, an amide, a thiol, a substituted alkyl, an unsubstituted alkyl, a sulfide, a substituted aryl, an unsubstituted aryl, or an indolizine, or R₅ is absent and (CR₃R₄)n is a substituted or unsubstituted alkyl that forms a ring with the nitrogen attached to C₂ position, and
when R₅ is or contains an amino group, a thiol group, and/or a hydroxyl group, the amino group, thiol group, and/or hydroxyl group is protected by a protecting group;
wherein each occurrence of the protecting group is independently acetyl, benzoyl, benzyl, methyl benzyl, β-methoxyethoxymethyl ether, dimethoxytrityl, methoxymethyl ether, methoxytrityl[(4-methoxyphenyl)diphenylmethyl], p-methoxybenzyl ether, p-methoxyphenyl ether, methylthiomethyl ether, pivaloyl, tetrahydropyranyl, tetrahydrofuran, trityl, silyl ether, methyl ether, ethoxyethyl ether, carbobenzyloxy, p-methoxybenzyl carbonyl, tert-butyloxycarbonyl, 9-fluorenylmethyloxycarbonyl, carbamate, p-methoxybenzyl, 3,4-dimethoxybenzyl, p-methoxyphenyl, tosyl, trichloroethyl chloroformate, nosyl, nps, methyl ester, benzyl ester, tert-butyl ester, 2,6-disubstituted phenol ester, silyl ester, orthoester, or oxazoline,
wherein the method comprises (i) performing a reaction between a first reactant of Formula (II) and a second reactant of Formula (III): 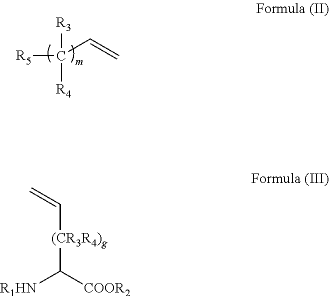
wherein each of m and g is an integer between 0 and 2 and R₁-R₅ are as defined above.

2. The method of claim 1, wherein the first reactant has the structure of Formula (IV): 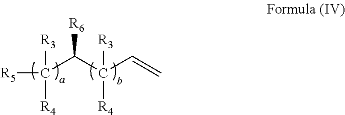
wherein each of a and b is independently 0 or 1;
wherein each occurrence of R₃ and R₄ is independently a hydrogen, a substituted alkyl, an unsubstituted alkyl, a hydroxyl, a thiol, or NR₁₃R₁₄, each of R₁₃ and R₁₄ is hydrogen, a substituted alkyl, or an unsubstituted alkyl;
wherein R₅ is a ketimine, an imidazole, an amino group, a carboxyl, a carboxylate, a hydroxyl, an amide, a thiol, a substituted alkyl, an unsubstituted alkyl, a sulfide, a substituted aryl, an unsubstituted aryl, or an indolizine, and
when R₅ is or contains an amino group, a thiol group, and/or a hydroxyl group, the amino group, thiol group, and/or hydroxyl group is protected by a protecting group selected from the group consisting of acetyl, benzoyl, benzyl, methyl benzyl, tert-butyloxycarbonyl, 9-fluorenylmethyloxycarbonyl, carbamate, carbobenzyloxy, p-methoxybenzyl, 3,4-dimethoxybenzyl, and p-methoxyphenyl; and
wherein R₆ is a substituted alkyl, an unsubstituted alkyl, a hydroxyl, a thiol, or NR₁₃R₁₄, each of R₁₃ and R₁₄ is hydrogen, substituted alkyl, or unsubstituted alkyl.

3. The method of claim 1, wherein the first reactant has the structure of Formula (V): 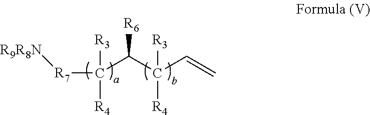
wherein each of a and b is independently 0 or 1;
wherein each occurrence of R₃ and R₄ is independently a hydrogen, a substituted alkyl, an unsubstituted alkyl, a hydroxyl, a thiol, or NR₁₃R₁₄, each of R₁₃ and R₁₄ is hydrogen, a substituted alkyl, or an unsubstituted alkyl;
wherein R₆ is a substituted alkyl, an unsubstituted alkyl, a hydroxyl, a thiol, or NR₁₃R₁₄, each of R₁₃ and R₁₄ is hydrogen, substituted alkyl, or unsubstituted alkyl;
wherein R₇ is absent, a hetero alkenyl that forms an imidazole with NR₈R₉, a carbonyl, 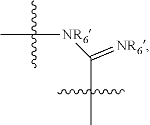
or an unsubstituted alkenyl that forms an indolizine with NR₈R₉, each occurrence of R₆′ is independently a hydrogen or a protecting group;
wherein each of R₈ and R₉ is independently absent, a hydrogen, or a protecting group; and
wherein each occurrence of the protecting group is independently acetyl, benzoyl, benzyl, methyl benzyl, tert-butyloxycarbonyl, 9-fluorenylmethyloxycarbonyl, carbamate, carbobenzyloxy, p-methoxybenzyl, 3,4-dimethoxybenzyl, or p-methoxyphenyl.

4. (canceled)

5. The method of claim 1, wherein the first reactant has the structure of Formula (VII): 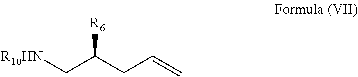
wherein R₆ is a hydroxyl, a thiol, or NR₁₃R₁₄, each of R₁₃ and R₁₄ is hydrogen, substituted alkyl, or unsubstituted alkyl; and
wherein R₁₀ is a hydrogen or a protecting group selected from the group consisting of acetyl, benzoyl, benzyl, methyl benzyl, tert-butyloxycarbonyl, 9-fluorenylmethyloxycarbonyl, carbamate, carbobenzyloxy, p-methoxybenzyl, 3,4-dimethoxybenzyl, or p-methoxyphenyl.

6. (canceled)

7. The method of claim 1, wherein the second reactant has the structure of Formula (IX): 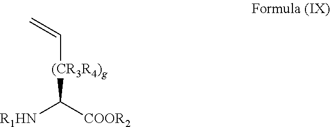
wherein g is an integer from 0 to 2;
wherein each of R₁ and R₂ is independently a protecting group selected from the group consisting of acetyl, benzoyl, benzyl, methyl benzyl, tert-butyloxycarbonyl, 9-fluorenylmethyloxycarbonyl, carbamate, carbobenzyloxy, p-methoxybenzyl, 3,4-dimethoxybenzyl, and p-methoxyphenyl; and
wherein each occurrence of R₃ and R₄ is independently a hydrogen, a substituted alkyl, an unsubstituted alkyl, a hydroxyl, a thiol, or NR₁₃R₁₄, each of R₁₃ and R₁₄ is hydrogen, a substituted alkyl, or an unsubstituted alkyl.

8-12. (canceled)

13. The method of claim 1, further comprising a hydrogenation step subsequent to step (i), or
step (i) is performed using a catalyst.

14. (canceled)

15. A method of making a glycosylated amino acid of Formula (XII): 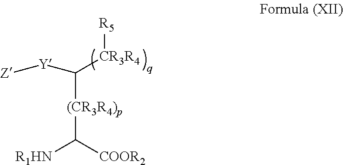
wherein each of p and q is an integer from 0 to 2;
wherein Z′ is a monosaccharide moiety, a disaccharide moiety, an oligosaccharide moiety, or a polysaccharide moiety;
wherein Y′ is an oxygen, a sulfur, or NR₁₅, R₁₅ is hydrogen, a substituted alkyl, or an unsubstituted alkyl;
wherein each of R₁ and R₂ is independently a hydrogen or a protecting group;
wherein each occurrence of R₃ and R₄ is independently a hydrogen, a substituted alkyl, an unsubstituted alkyl, a hydroxyl, a thiol, or NR₁₃R₁₄, each of R₁₃ and R₁₄ is hydrogen, a substituted alkyl, or an unsubstituted alkyl;
wherein R₅ is absent, a ketimine, an imidazole, an amino group, a carboxyl, a carboxylate, a hydroxyl, an amide, a thiol, a substituted alkyl, an unsubstituted alkyl, a sulfide, a substituted aryl, an unsubstituted aryl, or an indolizine, and
when R₅ is or contains an amino group, a thiol group, and/or a hydroxyl group, the amino group, thiol group, and/or hydroxyl group is protected by a protecting group;
wherein each occurrence of the protecting group is independently acetyl, benzoyl, benzyl, methyl benzyl, β-methoxyethoxymethyl ether, dimethoxytrityl, methoxymethyl ether, methoxytrityl[(4-methoxyphenyl)diphenylmethyl], p-methoxybenzyl ether, p-methoxyphenyl ether, methylthiomethyl ether, pivaloyl, tetrahydropyranyl, tetrahydrofuran, trityl, silyl ether, methyl ether, ethoxyethyl ether, carbobenzyloxy, p-methoxybenzyl carbonyl, tert-butyloxycarbonyl, 9-fluorenylmethyloxycarbonyl, carbamate, p-methoxybenzyl, 3,4-dimethoxybenzyl, p-methoxyphenyl, tosyl, trichloroethyl chloroformate, nosyl, nps, methyl ester, benzyl ester, tert-butyl ester, 2,6-disubstituted phenol ester, silyl ester, orthoester, or oxazoline,
wherein the method comprises (ii) performing a reaction between a saccharide of Formula (XIII) and an amino acid building block of Formula (I): 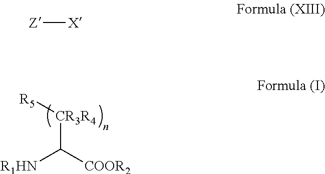
wherein Z′ and R₁-R₅ are as defined above;
wherein n is an integer from 0 to 4; and
wherein X′ is a leaving group.

16. The method of claim 15, wherein the saccharide has the structure of Formula (XIV) or Formula (XV): 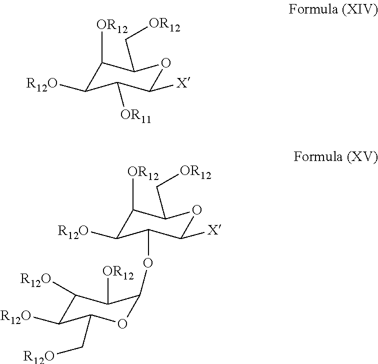
wherein each occurrence of R₁₁ and R₁₂ is independently a hydrogen or a protecting group selected from the group consisting of acetyl, benzoyl, benzyl, methyl benzyl, tert-butyloxycarbonyl, 9-fluorenylmethyloxycarbonyl, carbamate, carbobenzyloxy, p-methoxybenzyl, 3,4-dimethoxybenzyl, and p-methoxyphenyl.

17. The method of claim 15, wherein X′ is a dinitrogen, a dialkyl ether, a perfluoroalkylsulfonate, tosylate, mesylate, a halogen, SR₁₆, OR₁₇, a thioether, an amino group, a carboxylate, a phenoxide, or an amide, and
wherein each of R₁₆ and R₁₇ is independently a hydrogen, a substituted alkyl, an unsubstituted alkyl, a substituted aryl, an unsubstituted aryl, an imino, or a carbonyl.

18. (canceled)

19. The method of claim 15, wherein the amino acid building block has the structure of Formula (XVI): 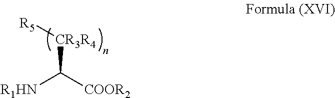
wherein n is an integer from 1 to 4;
wherein each of R₁ and R₂ is independently a protecting group;
wherein each occurrence of R₃ and R₄ is independently a hydrogen, a substituted alkyl, an unsubstituted alkyl, a hydroxyl, a thiol, or NR₁₃R₁₄, each of R₁₃ and R₁₄ is hydrogen, a substituted alkyl, or an unsubstituted alkyl;
wherein R₅ is a ketimine, an imidazole, an amino group, a carboxyl, a carboxylate, a hydroxyl, an amide, a thiol, a substituted alkyl, an unsubstituted alkyl, a sulfide, a substituted aryl, an unsubstituted aryl, or an indolizine, and
when R₅ is or contains an amino group, a thiol group, and/or a hydroxyl group, the amino group, thiol group, and/or hydroxyl group is protected by a protecting group; and
wherein each occurrence of the protecting group is independently acetyl, benzoyl, benzyl, methyl benzyl, tert-butyloxycarbonyl, 9-fluorenylmethyloxycarbonyl, carbamate, carbobenzyloxy, p-methoxybenzyl, 3,4-dimethoxybenzyl, or p-methoxyphenyl.

20. The method of claim 15, wherein the amino acid building block has the structure of Formula (XVII): 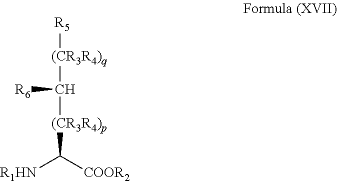
wherein each of p and q is an integer from 0 to 2 and p+q≤3;
wherein each of R₁ and R₂ is independently a protecting group;
wherein each occurrence of R₃ and R₄ is independently a hydrogen, a substituted alkyl, an unsubstituted alkyl, a hydroxyl, a thiol, or NR₁₃R₁₄, each of R₁₃ and R₁₄ is hydrogen, a substituted alkyl, or an unsubstituted alkyl;
wherein R₅ is a ketimine, an imidazole, an amino group, a carboxyl, a carboxylate, a hydroxyl, an amide, a thiol, a substituted alkyl, an unsubstituted alkyl, a sulfide, a substituted aryl, an unsubstituted aryl, or an indolizine, and
when R₅ is or contains an amino group, a thiol group, and/or a hydroxyl group, the amino group, thiol group, and/or hydroxyl group is protected by a protecting group;
wherein R₆ is a substituted alkyl, an unsubstituted alkyl, a hydroxyl, a thiol, or NR₁₃R₁₄, each of R₁₃ and R₁₄ is hydrogen, substituted alkyl, or unsubstituted alkyl; and
wherein each occurrence of the protecting group is independently acetyl, benzoyl, benzyl, methyl benzyl, tert-butyloxycarbonyl, 9-fluorenylmethyloxycarbonyl, carbamate, carbobenzyloxy, p-methoxybenzyl, 3,4-dimethoxybenzyl, or p-methoxyphenyl.

21-22. (canceled)

23. The method of claim 15, wherein the amino acid building block has the structure of Formula (XIX): 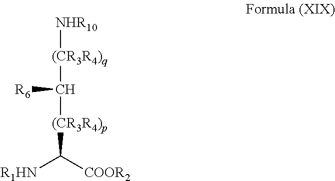
wherein each of p and q is an integer from 0 to 2 and p+q≤3;
wherein each of R₁ and R₂ is independently a protecting group;
wherein each occurrence of R₃ and R₄ is independently a hydrogen, a substituted alkyl, an unsubstituted alkyl, a hydroxyl, a thiol, or NR₁₃R₁₄, each of R₁₃ and R₁₄ is hydrogen, a substituted alkyl, or an unsubstituted alkyl;
wherein R₆ is a substituted alkyl, an unsubstituted alkyl, a hydroxyl, a thiol, or NR₁₃R₁₄, each of R₁₃ and R₁₄ is hydrogen, substituted alkyl, or unsubstituted alkyl;
wherein R₁₀ is a hydrogen or a protecting group; and
wherein each occurrence of the protecting group is independently acetyl, benzoyl, benzyl, methyl benzyl, tert-butyloxycarbonyl, 9-fluorenylmethyloxycarbonyl, carbamate, carbobenzyloxy, p-methoxybenzyl, 3,4-dimethoxybenzyl, or p-methoxyphenyl.

24-26. (canceled)

27. The method of claim 15, wherein step (ii) is performed in a solvent, optionally wherein the solvent is a mixture of dimethylformamide and dichloromethane, and/or wherein step (ii) is performed using a catalyst.

28-29. (canceled)

30. The method of claim 27, wherein the catalyst is a mixture of toluenesulfenyl chloride (TolSCl) and silver trifluoromethanesulfonate (AgOTf).

31-32. (canceled)

33. The method of claim 15, wherein the amino acid building block is produced by the method of claim 1.

34. A method for chemical synthesis of a glycopeptide comprising performing solid phase peptide synthesis to assemble a peptide of a desired sequence and incorporating one or more glycosylated amino acids of Formula (XII) into the peptide in one or more desired positions, wherein the one or more glycosylated amino acids of Formula (XII) are produced by the method of claim 15.

35. (canceled)

36. The method of claim 34, further comprising ligating two or more peptide fragments to form the glycopeptide, wherein the two or more peptide fragments are ligated using salicylaldehyde ester-mediated ligation at serine and/or threonine residues, wherein at least one of the peptide fragments were chemically synthesized by the method of claim 34.

37. A glycopeptide synthesized by the method of claim 34.

38. The glycopeptide of claim 37, wherein the glycopeptide comprises the amino acid sequence of any one of SEQ ID NOs:1-3 or a sequence having at least 75% sequence identity to any one of SEQ ID NOs:1-3.

39. An isolated glycopeptide comprising the collagenous domain of human adiponectin or a portion thereof, wherein the glycopeptide comprises one or more hydroxylysine residues in the collagenous domain;
wherein the glycopeptide comprises one or more glycosylated lysine residues in the collagenous domain; wherein the one or more glycosylated lysine residues are chemically synthesized according to the method of claim 15; and
wherein the human adiponectin comprises the amino acid sequence of SEQ ID NO:1.

40. The glycopeptide of claim 39, wherein the collagenous domain of human adiponectin comprises the amino acid sequence of SEQ ID NO:2.

41-42. (canceled)

43. The glycopeptide of claim 39, wherein the one or more glycosylated lysine residues are selected from the group comprising lysine residues 65, 68, 77, and 101 of human adiponectin.

44-47. (canceled)

48. The glycopeptide of claim 39, wherein the one or more glycosylated lysine residues are glycosylated with, independently for each of the one or more lysine residues, a glucosylgalactosyl moiety, a glucosylglucosyl moiety, a galactosylglucosyl moiety, or a galactosylgalactosyl moiety.

49. (canceled)

50. The glycopeptide of claim 39, further comprising the variable region of the human adiponectin or a portion thereof.

51. The glycopeptide of claim 50, wherein the glycopeptide comprises the
sequence
(SEQ ID NO: 3)
WMAGIPGHPGHNGAPGRDGRDGTPGEKGEKGDPGLIGPKGDIGETGVPG
AEGPRGFPGIQGRKGEPGEG.

52. The glycopeptide of claim 39, comprising the amino acid sequence of SEQ ID NO:1 or a sequence having at least 75% sequence identity to SEQ ID NO:1.

53. The glycopeptide of claim 39, wherein administration of the glycopeptide to a subject reduces cancer cell proliferation, viability, or metastasis, reduces tumor growth or tumor burden, reduces body weight or body fat mass, improves glucose tolerance, improves insulin sensitivity, reduces or inhibits gluconeogenesis, reduces triglyceride or cholesterol content/levels, reduces or inhibits inflammation, reduces the expression levels of one or more liver injury biomarkers, improves immune cell development and function, or combinations thereof.

54. The glycopeptide of claim 53, wherein:
the one or more liver injury biomarkers is selected from ALT, AST, TNFα, CCL2, LDLR, COL1, COL6, TBL, ALP, IL-6, and IL-10, or
the subject is suffering from obesity, cancer, steatohepatitis or other liver disease, a metabolic disease, Type 1 diabetes, Type 2 diabetes, obesity, metabolic syndrome, hypertension, atherosclerosis, inflammation, hyperglycemia, endothelial dysfunction, insulin resistance, or a combination thereof.

55. (canceled)

56. A pharmaceutical composition comprising the glycopeptide of claim 37 and a pharmaceutically acceptable carrier.

57-58. (canceled)

59. A method of treating a subject having a disease, disorder, or condition comprising administering to the subject an effective amount of the pharmaceutical composition of claim 56.

60. The method of claim 59, wherein:
the disease, disorder, or condition is associated with reduced or low adiponectin levels, or
the disease, disorder, or condition is selected from the group comprising cancer, steatohepatitis or other liver disease, Type 1 diabetes, Type 2 diabetes, obesity, metabolic syndrome, hypertension, atherosclerosis, inflammation, hyperglycemia, endothelial dysfunction, and insulin resistance.

61. (canceled)

62. The method of claim 59, wherein the composition is administered in an effective amount to reduce cancer cell proliferation or viability, reduce tumor growth or tumor burden, reduce body weight or body fat mass, improve glucose tolerance, improve insulin sensitivity, reduce or inhibit gluconeogenesis, reduces triglyceride or cholesterol, reduce or inhibit inflammation, reduce the expression levels of one or more liver injury biomarkers, or combinations thereof.

63. (canceled)

64. The glycopeptide of claim 39, wherein the glycopeptide comprises the sequence WMAGIPGHPGHNGAPGRDGRDGTPGEKGEKGDPGLIGPKGDIGETGVPGAEG PRGFPGIQGRKGEPGEG (SEQ ID NO:3), wherein the glycopeptide is glycosylated at lysine residues 65, 68, 77, and 101 of human adiponectin, wherein the glycosylating moieties are 2-O-α-D-glucopyranosyl-D-galactose moieties.

65. The glycopeptide of claim 64 comprising the structure:

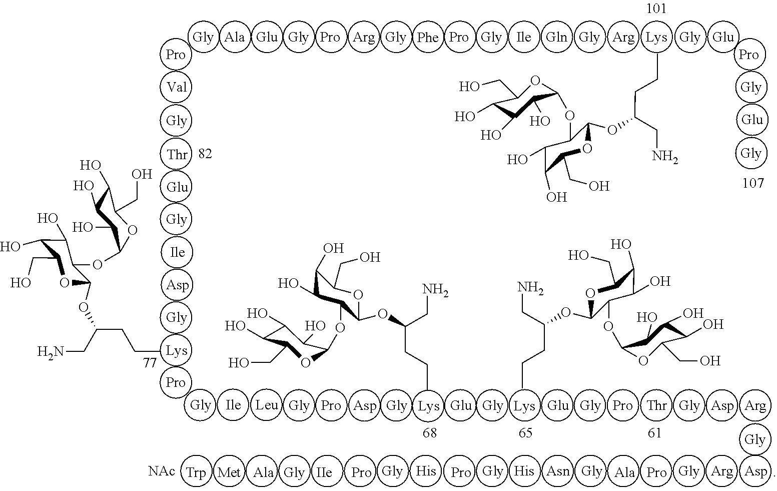

66. A glycopeptide produced by the method of claim 34, wherein the glycopeptide comprises the sequence WMAGIPGHPGHNGAPGRDGRDGTPGEKGE KGDPGLIGPKGDIGETGVPGAEGPRGFPGIQGRKGEPGEG (SEQ ID NO:3), wherein the glycopeptide is glycosylated at lysine residues 65, 68, 77, and 101 of human adiponectin, wherein the glycosylating moieties are 2-O-α-D-glucopyranosyl-D-galactose moieties.

67. The glycopeptide of claim 66 comprising the structure: [chemical structure]


SEQUENCE LISTING (5 sequences)

SEQ ID NO: 1 - protein, 244 aa, Homo sapiens
MLLLGAVLLLLALPGHDQETTTQGPGVLLPLPKGACTGWMAGIPGHPGHNGAPGRDGRDGTPGEKGEKGDPGLIGPKGDIGETGVPGAEGPRGFPGIQGRKGEPGEGAYVYRSAFSVGLETYVTIPNMPIRFTKIFYNQQNHYDGSTGKFHCNIPGLYYFAYHITVYMKDVKVSLFKKDKAMLFTYDQYQENNVDQASGSVLLHLEVGDQVWLQVYGEGERNGLYADNDNDSTFTGFLLYHDTN

SEQ ID NO: 2 - protein, 66 aa, Homo sapiens
GIPGHPGHNGAPGRDGRDGTPGEKGEKGDPGLIGPKGDIGETGVPGAEGPRGFPGIQGRKGEPGEG

SEQ ID NO: 3 - protein, 69 aa, Homo sapiens
WMAGIPGHPGHNGAPGRDGRDGTPGEKGEKGDPGLIGPKGDIGETGVPGAEGPRGFPGIQGRKGEPGEG

SEQ ID NO: 4 - protein, 47 aa, Artificial Sequence
  Synthetic peptide
TPGEKGEKGDPGLIGPKGDIGETGVPGAEGPRGFPGIQGRKGEPGEG

SEQ ID NO: 5 - protein, 22 aa, Artificial Sequence
  Synthetic peptide
WMAGIPGHPGHNGAPGRDGRDG